(19) United States
(12) Patent Application Publication
(10) Pub. No.: US 2026/0294910 A1
(43) Pub. Date: Oct. 1, 2026

(54) ORAL PHARMACEUTICAL COMPOSITIONS

(71) Applicant: Assembly Biosciences, Inc., South San Francisco, CA (US)

(72) Inventors: Zhixin ZONG, South San Francisco, CA (US); Nicole WHITE, South San Francisco, CA (US); Yi WU, South San Francisco, CA (US)

(73) Assignee: Assembly Biosciences, Inc., South San Francisco, CA (US)

(21) Appl. No.: 19/477,816

(22) PCT Filed: Apr. 24, 2024
(86) PCT No.: PCT/IB2024/053990

§ 371 Date: Oct. 23, 2025

Related U.S. Application Data
(60) Provisional application No. 63/498,398, filed on Apr. 26, 2023.
(60) Provisional application No. 63/522,996, filed on Jun. 23, 2023.
(60) Provisional application No. 63/553,453, filed on Feb. 14, 2024.

(51) Int. Cl. A61K 31/51; A61K 9/16; A61P 31/22

(52) U.S. Cl. CPC A61K 31/51; A61K 9/1652; A61P 31/22

(57) ABSTRACT

The present invention relates to methods and compositions for treating and/or inhibiting the development or progression of diseases or disorders caused by, or associated with, herpes virus infection. In particular, provided are oral high bioavailability and long-acting pharmaceutical compositions comprising Compound 1; methods for their manufacture; and the use of said pharmaceutical compositions as a medicament and for the treatment of diseases or disorders caused by, or associated with, herpes virus.

### FIELD OF THE INVENTION

Provided herein are compositions and methods for treating and/or inhibiting the development or progression of diseases or disorders caused by, or associated with, herpes virus infection. In particular, provided herein are oral high bioavailability and long-acting pharmaceutical compositions comprising a potent helicase-primase inhibitor, namely 2-(3-(2′,5′-difluoro-[1,1′-biphenyl]-4-yl)-2-oxotetrahydropyrimidin-1(2H)-yl)-4-methylthiazole-5-sulfonamide (also referred to herein as ‘Compound 1’), or a pharmaceutically acceptable salt thereof; methods for their manufacture; and the use of said pharmaceutical compositions as a medicament and for the treatment of diseases or disorders caused by, or associated with, herpes virus.

### BACKGROUND OF THE INVENTION

Human herpes viruses are large-enveloped double-stranded DNA viruses that share the characteristic of establishing life-long infections in humans. This is accomplished by their ability to exist in the host either as a symptom free latent infection, where the virus lies dormant or, following activation, as a lytic infection with associated symptoms. These viral infections have widespread, worldwide prevalence and it is notable that over 90% of all humans are chronically infected with more than one human herpes virus.

Human herpes viruses are classified into three subfamilies (a, B and y) based upon their biological characteristics and the family consists of eight members, i.e., Herpes Simplex Virus subtype type 1 and 2 (HSV1, HSV2), Varicella Zoster Virus (VZV), Epstein-Barr virus (EBV), Cytomegalovirus (CMV), and Human Herpes Viruses 6-8 (HHV 6-8).

HSV1 and 2 infections can cause disease in immune competent individuals. Both subtypes cause cutaneous genital/anal and oro-labial/nasal cavity (cold sore) lesions, although HSV2 is more commonly associated with the former and HSV1 the latter. It is believed that >80% of genital infections are caused by HSV2. Globally, over 500 million people have genital herpes infections and approximately 50 to 80% of the world's population have oro-labial HSV infection, which is the main cause of cold sores. HSV, and particularly HSV1, can also cause lesions on the fingers (Whitlows) and other areas of the skin.

The vast majority of HSV infected individuals will not experience any noticeable symptoms. However, some will experience recurrent (and often severe) outbreaks of infection. In the USA, 20 to 40% of the population will get recurrent labial HSV lesions. Significantly, oro-labial cold sores and Whitlow's provide a very easy route for transmission of the virus to other individuals which can lead to rarer but much more serious HSV-related pathologies. For example, HSV-related ocular keratitis is a major cause of blindness and HSV can also cause encephalitis in neonates, which is a life-threatening condition. Other disorders believed to be caused by HSV include herpes gladiatorum, Mollaret's meningitis and possibly Bell's palsy.

Primary infection with, or reactivation of an existing herpes virus infection, can be a major cause of disease in immunocompromised individuals. Key at-risk populations include patients undergoing solid organ or stem cell transplantation, patients undergoing cancer treatment, individuals with HIV/AIDS, and ICU patients.

Presently, there is no cure for HSV. Medicines have been developed that can to some degree reduce the occurrence and/or shorten the length of outbreaks, but there is a need for improved therapies.

Currently, nucleoside analogues, such as acyclovir and its prodrugs, e.g., valacyclovir and famciclovir, are used as agents against herpes viruses such as HSV. In order to exert their effects, these nucleoside analogues must be phosphorylated by viral thymidine kinase (TK) and subsequently converted by cellular kinases to the nucleoside triphosphate, which inhibits the activity of the viral DNA polymerase. If the virus has no functionally active TK, as is the case, for example, with resistant HHV1 mutants or with TK-negative viruses, the nucleoside analogues are unable to exert their effects.

Nucleoside analogues are clinically administered at very high doses, e.g., doses as high as several hundred milligrams to several grams are typically administered per day. Even at these high doses, which are often administered over long treatment durations, these drugs are unable to completely prevent recurrent outbreaks of symptoms from HSV infection. Nucleoside analogues also do little to address the issue of viral shedding, which can asymptomatically facilitate the transmission of HSV to more individuals. Certain nucleoside analogues, particularly when used at high doses, also give rise to safety concerns. For example, since these agents can incorporate into the genome DNA of a host via the host DNA polymerase, their mutagenicity is of concern, as documented for the nucleoside analogue, ganciclovir (Aoki, Chapter 45 in Mandell, Douglas and Bennett's Principles and Practice of Infectious Diseases (Eighth Edition) 2015).

Given the inadequacy of existing treatments, there is an urgent medical need to develop improved, well-tolerated anti-herpes treatments.

One class of compounds currently being investigated are the helicase-primase inhibitors. Helicase-primase inhibitors are antiviral agents with a novel mechanism of action. They inhibit the viral heterotrimeric complex consisting of helicase, primase, and cofactor subunits, which have functions that are essential for viral DNA replication. These agents are not nucleoside analogues and do not require phosphorylation by TK to inhibit HSV replication and they are therefore potentially active against TK-deficient HSV, which as described above, is a major mechanism of resistance to nucleoside analogues.

Two examples of helicase-primase inhibitors are BILS-179 BS (Crute et al., (2002) Nature Medicine 8, p. 386-391) and amenamevir (Katsumata et al. (2018) Biochem Pharm 158 p. 201-206).

BILS-179 BS has been dosed orally to humans but was suspended in early clinical trials due to adverse events (Ruebsamen et al., (2019) Med. Chem. Commun., DOI: 10.1039/C9MD00233B). Similarly, amenamevir, which has also been dosed orally to humans, was suspended from early HSV clinical trials due to adverse events. In a dose-finding, placebo-controlled study with 437 patients with recurrent genital herpes, amenamevir was administered orally at one of four doses, namely 100 mg, 200 mg, 400 mg, and 1200 mg. It was found that the time to lesion healing, i.e., the primary endpoint of the study, was only significantly different between the very highest 1200 mg single dose group and the placebo-tested group (Aoki, Chapter 45 in Mandell, Douglas and Bennett's Principles and Practice of Infectious Diseases (Eighth Edition) 2015).

Another example of a helicase-primase inhibitor is pritelivir, a thiazolylamide derivative with the chemical name N-Methyl-N-(4-methyl-5-sulfamoyl-1,3-thiazol-2-yl)-2-[4-(pyridin-2-yl)phenyl]acetamide. The compound has been disclosed in WO 2001/47904.

In a human clinical study, the effect of pritelivir on suppression of genital herpes was studied in 156 individuals (Wald et al., (2014) New England Journal of Medicine 370, p. 201-210). Subjects received one of three oral daily doses or one weekly oral dose of pritelivir or placebo for 28 days. The four pritelivir dosing regimens were; a loading dose of 20 mg followed by a daily dose of 5 mg; a loading dose of 100 mg followed by a daily dose of 25 mg; a loading dose of 300 mg followed by a daily dose of 75 mg; and a weekly dose of 400 mg. At high doses, it was found that pritelivir reduced the rate of genital HSV shedding, the primary end point for the study. Pritelivir also reduced the number of days with lesions in otherwise healthy men and women with genital herpes. An oral daily dose of 75 mg had the greatest antiviral effect and this was found to be superior to the less frequently administered weekly dose of 400 mg.

As highlighted in the study report, although the HSV shedding rate was reduced at the highest daily dose of 75 mg, as compared with placebo, break-through shedding remained. When discussing this finding, it is explained in Wald et al., ((2014) New England Journal of Medicine 370, p. 201-210) that persistent, low-level shedding has also been observed with nucleoside therapy and that the pathogenesis of break-through viral shedding during adequate antiviral therapy with nucleoside analogues is poorly understood; it is not related to lack of adherence to the treatment regimen or viral resistance. The author also raises the question of whether further increases in the daily dose of pritelivir would completely abrogate viral shedding and should be addressed in subsequent clinical studies.

In accordance with this suggestion, a subsequent clinical study using an even higher daily oral dose was indeed conducted. In a Phase II 28-day clinical study of 91 subjects with recurrent genital HSV-2, daily oral dosing of 100 mg pritelivir (after a loading dose of 400 mg) resulted in HSV shedding in 2.4% of genital swabs analysed, compared with HSV shedding in 5.3% of swabs following daily dosing of 500 mg of valacyclovir. The results of the study are reported in Wald et al., (2016) (J. Am. Med. Assoc. 316(23), p. 2495-2503) and the publication mentions that the 100 mg pritelivir daily dose (after a loading dose of 400 mg) was chosen based on prior trial findings showing high efficacy of the 75 mg daily dose in suppressing viral shedding.

In a current phase III clinical trial, a 400 mg oral loading dose, followed by 100 mg daily oral dosing of pritelivir for up to 28 days or until all mucocutaneous HSV lesions are healed, whichever is earlier, is being investigated (https://www.clinicaltrials.gov/ct2/show/NCT03073967?term=pritelivir+f&draw=2&rank=2)

As is apparent from the above, efforts to improve therapy with the small molecule based antiviral therapeutics described above, e.g. to try and further reduce or prevent HSV shedding and viral reactivation, have largely focussed on using higher oral drug doses (including high loading doses) and more frequent dosing (e.g. multiple daily dosing).

There is still an ongoing need for novel and improved methods for treating HSV infections and the present invention was devised with the foregoing in mind.

### SUMMARY OF THE INVENTION

In a first aspect, the present invention provides an oral pharmaceutical composition comprising a solid dispersion, wherein the solid dispersion comprises a compound (Compound 1) represented by:

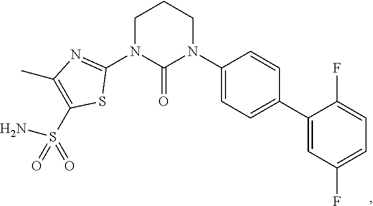 [SMILES: Cc1nc(N2CCCN(c3ccc(-c4cc(F)ccc4F)cc3)C2=O)sc1S(N)(=O)=O]

or a pharmaceutically acceptable salt thereof, and at least one matrix polymer.

It has been surprisingly found that when Compound 1, or a pharmaceutically acceptable salt thereof, is formulated as a pharmaceutical composition comprising a solid dispersion in accordance with the present invention, such a composition provides beneficial properties, including a high bioavailability and/or certain release profiles that allow rapid high plasma levels to be achieved and maintained for prolonged periods. Remarkably, in some embodiments, the compositions of the present invention are able, e.g. after steady-state levels have been attained, to provide steady and continuous high plasma levels of Compound 1 for at least 14 days after administration. In some embodiments, compositions of the present invention are able to provide steady and continuous high plasma levels of Compound 1 for a prolonged period after just a single oral administration, i.e. without the need for a loading dose. It has also been surprisingly found that certain compositions of the present invention are able to provide even higher plasma levels of the helicase-primase inhibitor for a prolonged period when administered in connection with the intake of food.

The above-mentioned benefits offer the possibility for much less frequent dosing and/or the use of lower doses than other forms of administration. This is an unexpected finding. As described above, efforts to improve therapy for small molecule based antiviral therapeutics have largely focussed on increasing oral drug doses (including high loading doses) and using more frequent drug dosing regimens (e.g. multiple daily dosing).

The present invention satisfies a need for a novel treatment approach for HSV infections which can provide improvements in efficacy and/or safety and/or patient use.

In a second aspect, the present invention provides a method for forming a pharmaceutical composition according to the first aspect. Various techniques may be used for forming the compositions of the present invention, however, it has been found that certain processes offer particular advantages. For example, various techniques may be used for forming solid dispersions, however, it has been found that spray drying offers particular advantages.

In a third aspect, the present invention provides a method for treating or preventing a herpes infection in a subject in need thereof, the method comprising administering Compound 1 or a pharmaceutically acceptable salt thereof, to the subject, wherein the Compound 1 or a pharmaceutically acceptable salt thereof, is formulated as a pharmaceutical composition according to the first aspect.

### BRIEF DESCRIPTION OF THE DRAWINGS

The summary, as well as the following detailed description, is further understood when read in conjunction with the appended drawings. For the purpose of illustrating the disclosed compositions and methods, there are shown in the drawings exemplary embodiments of the compositions and methods; however, the compositions and methods are not limited to the specific embodiments disclosed. In the drawings:

FIG. 1 shows the mean plasma concentration-time profiles of Compound 1 after intravenous dosing of the solution formulations disclosed in Example 3 (open triangles) at 1 mg/kg in male sprague-dawley rats (n=3).

FIG. 2 shows the mean plasma concentration-time profiles of Compound 1 after intravenous dosing of the solution formulations disclosed in Example 3 (open triangles) at 0.25 mg/kg in male cynomolgus monkeys (n=3).

FIG. 3 shows the mean plasma concentration-time profiles of Compound 1 after intravenous dosing of the solution formulations disclosed in Example 3 (open triangles) at 0.15 mg/kg in male non-naïve Beagle dogs (n=3).

FIG. 4 shows the XRPD diffractogram of Lot 1 of the Compound 1 solid dispersion formulation described in Example 4 and Table 17.

FIG. 5 shows the PLM image for Lot 1 of the Compound 1 solid dispersion formulation described in Example 4 and Table 17.

FIG. 6 shows DSC thermal analysis results of Lot 1 of the Compound 1 solid dispersion formulation described in Example 4 and Table 17.

FIG. 7 shows TGA analysis of Lot 1 of the Compound 1 solid dispersion formulation described in Example 4 and Table 17.

FIG. 8 shows the XRPD diffractogram of Lot 2 of the Compound 1 solid dispersion formulation described in Example 4 and Table 17.

FIG. 9 shows the PLM image for Lot 2 of the Compound 1 solid dispersion formulation described in Example 4 and Table 17.

FIG. 10 shows DSC thermal analysis results of Lot 2 of the Compound 1 solid dispersion formulation described in Example 4 and Table 17.

FIG. 11 shows TGA analysis of Lot 2 of the Compound 1 solid dispersion formulation described in Example 4 and Table 17.

FIG. 12 shows the XRPD diffractogram of Lot 3 of the Compound 1 solid dispersion formulation described in Example 4 and Table 17.

FIG. 13 shows the PLM image for Lot 3 of the Compound 1 solid dispersion formulation described in Example 4 and Table 17.

FIG. 14 shows DSC thermal analysis results of Lot 3 of the Compound 1 solid dispersion formulation described in Example 4 and Table 17.

FIG. 15 shows TGA analysis of Lot 3 of the Compound 1 solid dispersion formulation described in Example 4 and Table 17.

FIG. 16 shows the XRPD diffractogram of Lot 4 of the Compound 1 solid dispersion formulation described in Example 4 and Table 17.

FIG. 17 shows the PLM image for Lot 4 of the Compound 1 solid dispersion formulation described in Example 4 and Table 17.

FIG. 18 shows the DSC thermal analysis results of Lot 4 of the Compound 1 solid dispersion formulation described in Example 4 and Table 17.

FIG. 19 shows the TGA analysis of Lot 4 of the Compound 1 solid dispersion formulation described in Example 4 and Table 17.

FIG. 20 shows the mean plasma concentration-time profile of the compositions described in Example 5 after oral administration of the 90% NMP/10% TPGS solution (closed squares), the nanosuspension (open squares) and the SDD suspension (crosses) formulations disclosed in Example 5 in male sprague-dawley rats (n=3).

FIG. 21 shows the mean plasma concentration-time profile of the compositions described in Example 6 after oral administration of the 85% PEG300/10% TPGS/5% EtOH solution (triangles) and the SDD suspension formulation (diamonds) disclosed in Example 6 in male non-naïve Beagle dogs (n=3).

FIG. 22 and FIG. 23 show the mean plasma concentration-time profiles at Day 1 and Day 7 for the compositions described in Example 7 after oral administration at doses of 10 mg/kg (closed solid circles), 100 mg/kg (open squares) and 300 mg/kg (closed solid triangles) in male sprague-dawley rats (n=3).

FIG. 24 and FIG. 25 show the mean plasma concentration-time profiles at Day 1 and Day 7 for the compositions described in Example 8 after oral administration at doses of 1 mg/kg (closed solid circles), 10 mg/kg (open squares) and 100 mg/kg (closed solid triangles) in male non-naïve Beagle dogs (n=3).

FIG. 26 and FIG. 27 show the mean plasma concentration-time profiles at Day 1 and Day 14 for the compositions described in Example 9 after oral administration at doses of 15 mg/kg in male (closed solid circles) and female (open squares) non-naïve Beagle dogs (n=2).

FIG. 28 shows the mean plasma concentration-time profiles after 14 days consecutive oral dosing for the compositions described in Example 9 at doses of 15 mg/kg in male (open squares) and female (open circles) in non-naïve Beagle dogs (n=2 for each sex group).

FIG. 29 shows the mean plasma concentration-time profiles (n=3) of Compound 1 after intravenous dosing of the solution formulations disclosed in Example 10 in male non-naïve Beagle dogs at 0.10 mg/kg of Compound 1 with (closed diamonds) and without (open squares) oral charcoal administration.

FIG. 30 shows the XRPD diffractogram of Lot 1 of the Compound 1 solid dispersion formulation described in Example 11 and Table 20.

FIG. 31 shows the DSC thermal analysis results of Lot 1 of the Compound 1 solid dispersion formulation described in Example 11 and Table 20.

FIG. 32 shows the SEM image for Lot 1 of the Compound 1 solid dispersion formulation described in Example 11 and Table 20.

FIG. 33 shows the XRPD diffractogram of Lot 1 of the Compound 1 solid dispersion formulation described in Example 11 and Table 20, initial and after being stressed at 40° C./75% RH Open conditions for 5 days.

FIG. 34 shows the mean plasma concentration-time profile of the SDD suspension formulation composition described in Example 11 after oral administration to male non-naïve Beagle dogs (n=3) under fasted overnight (open squares) and fed (closed squares) conditions.

FIG. 35 shows the mean plasma concentration-time profiles of Compound 1 after IV dosing of the solution formulations disclosed in Example 3A to (A) male Sprague Dawley rats (n=3) at 0.2 mg/kg; (B) male non-naïve Beagle dogs (n=3) at 0.15 mg/kg; (C) male non-naïve cynomolgus monkeys (n=3) at 0.2 mg/kg; and (D) naïve Bama mini-pigs (n=3) at 0.25 mg/kg.

FIG. 36 shows the mean plasma concentration-time profile of the Tablet composition described in Example 13 after oral administration of 100 mg (2×50 mg tablets) to male non-naïve Beagle dogs under fasted pentagastrin pre-treatment (open triangles), fasted famotidine pre-treatment (open circles) or fed (open squares) conditions (n=3 per arm).

FIG. 37 shows the mean plasma concentration-time profiles of the Tablet composition described in Example 13 (closed squares) after oral administration of 100 mg (2×50 mg tablet) and the SDD suspension formulation (closed circles) disclosed in Example 12 after oral administration of 10 mg/kg to male non-naïve Beagle dogs under fasted conditions (n=3 per arm).

FIG. 38 shows the mean plasma concentration-time profiles of the Tablet composition described in Example 13 (closed squares) after oral administration of 100 mg (2×50 mg tablet) and the SDD suspension formulation (closed circles) disclosed in Example 12 after oral administration of 10 mg/kg to male non-naïve Beagle dogs under fed conditions (n=3 per arm).

FIG. 39 shows the XRPD diffractograms of the Compound 1 solid dispersion formulations described in Example 15.

FIG. 40 shows the XRPD diffractograms of the Compound 1: HPMCAS-HG solid dispersion formulation described in Example 15 after stressed stability at 40° C./75% RH open conditions for five days.

FIG. 41 shows the XRPD diffractograms of the Compound 1: Soluplus solid dispersion formulation described in Example 15 after stressed stability at 40° C./75% RH open conditions for five days.

FIG. 42 shows the XRPD diffractograms of the Compound 1: HPMCAS-LG solid dispersion formulation described in Example 15 after stressed stability at 40° C./75% RH open conditions for five days.

FIG. 43 shows the XRPD diffractograms of the Compound 1: HPMCAS-MG solid dispersion formulations described in Example 15 after stressed stability at 40° C./75% RH open conditions for five days.

FIG. 44 shows Compound 1 concentrations in the donor chamber of the Pion μflux device for the Compound 1 solid dispersion formulations described in Example 15.

FIG. 45 shows Compound 1 concentrations in the acceptor chamber of the Pion μflux device for the Compound 1 solid dispersion formulations described in Example 15.

FIG. 46 shows the mean plasma concentration-time profiles of the SDD suspension formulation composition described in Example 16 after oral administration to male non-naïve Beagle dogs (n=3).

### DETAILED DESCRIPTION OF THE INVENTION

The disclosed compositions, uses thereof and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

### Definitions

Unless otherwise stated, the following terms used in the specification and claims have the following meanings set out below.

Reference to a particular numerical value includes at least that particular value unless the context clearly dictates otherwise. When a range of values is expressed, another embodiment includes from the one particular value and/or to the other particular value. Further, reference to values stated in ranges include each and every value within that range. All ranges are inclusive and combinable.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any sub-combination.

As used herein, the singular forms “a,” “an,” and “the” include the plural.

As used herein, Cₘₐₓ refers to the geometric mean maximum concentration of the active agent. This may be measured in vivo following administration of a composition of the invention to a subject and measuring the plasma levels of the drug at various timepoints after dosing.

As used herein, AUC refers to the area under the curve and is the definite integral of the concentration of the active agent in blood plasma as a function of time.

As used herein, the term “amorphous” refers to a solid material having no long-range order in the position of its molecules. Amorphous solids are substances in which the molecules are arranged in a random manner so that there is no well-defined arrangement, e.g., molecular packing, and no long-range order. Amorphous solids are generally isotropic, i.e., exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid.

As used herein, the expression “substantially amorphous” refers to a solid material having little or no long-range order in the position of its molecules. For example, substantially amorphous materials have less than about 15% crystallinity (e.g., less than about 10% crystallinity or less than about 5% crystallinity). It is also noted that the term “substantially amorphous” includes the descriptor, “amorphous”, which refers to materials having no (0%) crystallinity. Conveniently, the substantially amorphous material has less than about 5% crystallinity.

As used herein, the term “dispersion” refers to a disperse system in which one substance, the dispersed phase, is distributed, in discrete units, throughout a second substance (the continuous phase or vehicle or carrier). The size of the dispersed phase can vary considerably (e.g., single molecules or colloidal particles of nanometer dimension up to multiple microns in size). In general, the dispersed phases can be solids, liquids, or gases. In the case of a solid dispersion, the dispersed and continuous phases are both solids. In pharmaceutical applications, a solid dispersion can include: an amorphous drug in an amorphous polymer; an amorphous drug in a crystalline polymer; a crystalline drug in an amorphous polymer; or a crystalline drug in crystalline polymer. Herein, a solid dispersion can include an amorphous drug in an amorphous polymer, an amorphous drug in a crystalline polymer, or a crystalline drug in an amorphous polymer. In some embodiments, a solid dispersion includes the matrix polymer constituting the dispersed phase, and the drug or compound constitutes the continuous phase. Alternatively, a solid dispersion includes the drug constituting the dispersed phase, and the matrix polymer constitutes the continuous phase or carrier. Conveniently, the solid dispersion of the present invention comprises a dispersed phase, which comprises Compound 1 or a pharmaceutically acceptable salt thereof, and a continuous phase, which comprises the at least one matrix polymer. More conveniently, the solid dispersion comprises amorphous Compound 1 or a pharmaceutically acceptable salt thereof in an amorphous matrix polymer.

As used herein, “matrix polymer” refers to a polymer suitable for use in a solid dispersion according to the present invention and comprises inert, pharmaceutically acceptable polymers. Suitable matrix polymers include linear, branched or cyclic, natural or synthetic homopolymers (e.g., polysaccharides) and copolymers (e.g., block copolymers).

As used herein, the term “povidone polymer” refers to polyvinylpyrrolidone or a derivative thereof, such as cross-linked polyvinylpyrrolidone, or crospovidone.

As used herein, the term “copovidone polymer” refers to a copolymer of vinylpyrrolidone and vinyl acetate, or a derivative thereof. An example of a copovidone polymer is PVP-VA64.

As used herein, the term “methacrylate polymer” refers to a polymer of methacrylic acid and/or methyl methacrylate. This includes anionic copolymers, as depicted below, which are a polymerization product of methacrylic acid and methyl methacrylate-p (MAA-co-MMA). The dissolution pH of p (MAA-co-MMA) is dictated by the ratio of monomers utilized in the polymerization. For example, a 1:1 molar ratio of methyl methacrylate and methacrylic acid results in a dissolution above pH 6.0. The resulting polymer is designated as Type A. Type B, synthesized using a 2:1 molar ratio of methyl ester and carboxylic acid monomers, results in a dissolution pH of >7.0. The chemical structure of p (MAA-co-MMA): n:m=1:1 (type A); n:m=2:1 (type B) is:

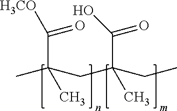

Poly(methacrylic acid-co-methyl methacrylate) polymers are marketed, for example, by Evonik Industries under the EUDRAGIT® tradename. As used herein, the term “EUDRAGIT® L100” refers to an anionic 1:1 methacrylic acid-methyl methacrylate copolymer (CAS number 25086-15-1), which dissolves in water above pH 6, and has a weight average molecular mass of approximately 125,000 g/mol.

As used herein, the term “HPMCAS” refers to hydroxypropylmethylcellulose acetate succinate (CAS 71138-97-1). HPMCAS is typically made from HPMC by esterification with acetic acid anhydride and succinic acid anhydride in acetic acid using a basic catalyst such as sodium acetate. The resulting product, as depicted below, is precipitated by addition of water and subsequently purified by washing with additional water. This reaction sequence leads to a plurality of hydrophobic sites and hydrogen bond acceptor and donor capability. The chemical structure of HPMCAS is:

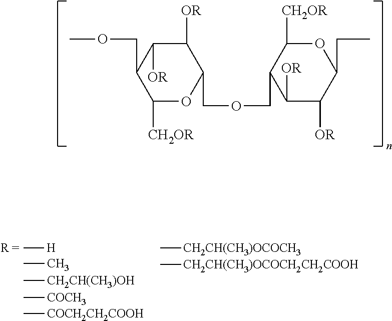

HPMCAS was first introduced by Shin-Etsu Chemical Co., Ltd., Japan, as an enteric coating agent with three substitution levels designated according to the content of acetyl substituents as L, M, or H (e.g., Shin-Etsu AQOAT® LF, MF, HF, LG, MG and HG) The dissolution pH of HPMCAS ranges from about 5.5 (L) to about 6.5 (H) depending on the buffer type used for dissolution. Dow Chemical also markets HPMCAS (e.g., Dow AFFINISOL® 716, 912 and 126) as well as Ashland Chemical (e.g., AQUASOLVE® L, M and H grades). In contrast to HPMC, where substitution levels are specified by the monographs, the range for HPMCAS is not limited to the three commercially available subranges. Manufacturer's specs for these products are shown below in Tables A-C.

TABLE A
Manufacturer's Specs for AQOAT ® HPMCAS by Shin-Estu

| | AS-LG | AS-MG | AS-HG |
|---|---|---|---|
| | AS-LF | AS-MF | AS-HF |
| Viscosity (mm2/s) | 2.4-3.6 | 2.4-3.6 | 2.4-3.6 |
| Heavy metals | ≤10 ppm | ≤10 ppm | ≤10 ppm |
| Arsenic | ≤2 ppm | ≤2 ppm | ≤2 ppm |
| Free succinic acid | ≤1.0% | ≤1.0% | ≤1.0% |
| Loss on drying | ≤5.0% | ≤5.0% | ≤5.0% |
| Residue on ignition | ≤0.20% | ≤0.20% | ≤0.20% |
| Methoxy content | 20.0-24.0% | 21.0-25.0% | 22.0-26.0% |
| Hydroxypropoxy content | 5.0-9.0% | 5.0-9.0% | 6.0-10.0% |
| Acetyl content | 5.0-9.0% | 7.0-11.0% | 10.0-14.0% |
| Succinoyl content | 14.0-18.0% | 10.0-14.0% | 4.0-8.0% |

TABLE B
Manufacturer's Specs for AFFINISOL ® HPMCAS products by Dow

| | 716 | 912 | 128 |
|---|---|---|---|
| Hydroxypropyl | 5.0-9.0% | 5.0-9.0% | 6.0-10.0% |
| Methoxyl | 20-24% | 21-25% | 22-26% |
| viscosity* (cP) | 2.4-3.6 | 2.4-3.6 | 2.4-3.6 |
| Residue on ignition | <0.20% | <0.20% | <0.20% |
| Loss on drying | <5.0% | <5.0% | <5.0% |
| Free acids | <1.0% | <1.0% | <1.0% |
| Acetate substitution | 5.0-9.0% | 7.0-11.0% | 10.0-14.0% |
| Succinate substitution | 14.0-18.0% | 10.0-14.0% | 4.0-8.0% |
| Acetic acid | 0.5% | 0.5% | 0.5% |

*viscosity determined as a 2% solution in NaOH solution

TABLE C
Manufacturer's Specs for AQUASOLVER HPMCAS products by Ashland

| | LF & LG | MF &MG | HF & HG |
|---|---|---|---|
| Viscosity* (mPa*s) | 2-4-3.6 | 2-4-3.6 | 2-4-3.6 |
| Loss on drying | ≤5% | ≤5% | ≤5% |
| Residue on ignition | ≤0.20% | ≤0.20% | ≤0.20% |
| Heavy metals | <10 ppm | <10 ppm | <10 ppm |
| Arsenic | ≤2 ppm | ≤2 ppm | ≤2 ppm |
| Limit of free succinic and | ≤1.0% | ≤1.0% | ≤1.0% |
| acetic acids | | | |
| Acetyl content | 5-9% w/w | 7-11% w/w | 10-14% w/w |
| Succinoyl content | 14-18% w/w | 10-14% w/w | 4-8% w/w |
| Methoxyl content | 20-24% w/w | 21-25% w/w | 22-26% w/w |
| Hydroxypropoxy content | 5-9% w/w | 5-9% w/w | 6-10% w/w |

*measured for a 2% solution at 20° C.

The properties of HPMCAS may be affected in particular by the acetyl and succinoyl contents of the polymer. Determination of the % w/w acetyl and succinoyl contents may be carried out by ester group hydrolysis of a weighed amount of polymer with 1M NaOH, followed by determination of the acetyl and succinoyl contents in the hydrolysed solutions by reverse-phase liquid chromatography against standard calibration solutions; this methodology is described in detail in Chen et al., Journal of AOAC International (2002), 85(4), 824-831, the contents of which are incorporated by reference.

As used herein the term “HPMCP” refers to hydroxypropyl methylcellulose phthalate (CAS 9050-31-1). The chemical structure of HPMCP, as depicted below, is a phthalic half ester of hydroxypropyl methylcellulose. The threshold pH value for rapid disintegration of HPMCP can be controlled by varying the phthalyl content. HPMCP is marketed, for example, by Shin-Etsu (e.g., HP-55 and HP-50 and HP-55S). Manufacturer's specs for these products are shown below in Table D. The chemical structure of HPMCP is:

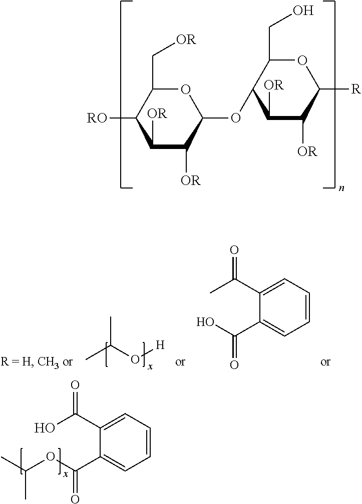

TABLE D
Manufacturer's Specs for HPMCP products by Shin-Etsu

| | HP-55 | HP-55S | HP-50 |
|---|---|---|---|
| Labelled viscosity (cst) | 40 | 170 | 55 |
| Viscosity (cst) | 32-48 | 136-204 | 44-66 |
| Water | ≤5.0% | ≤5.0% | ≤5.0% |
| Residue on ignition | ≤0.20% | ≤0.20% | ≤0.20% |
| Chloride | ≤0.07% | ≤0.07% | ≤0.07% |
| Heavy metals | ≤0.001% | ≤0.001% | ≤0.001% |
| Free phthalic acid | ≤1.0% | ≤1.0% | ≤1.0% |
| Phthalyl content | 27.0-35.0% | 27.0-35.0% | 21.0-27.0% |
| Methoxy content | 18.0-22.0% | 18.0-22.0% | 20.0-24.0% |
| Hydroxypropoxyl content | 5.0-9.0% | 5.0-9.0% | 6.0-10.0% |

As used herein, “patient” or “subject” refers to a mammal, including a domestic animal, animal kept as livestock and a zoo animal. Conveniently, the “patient” or “subject” is a human being.

It is to be appreciated that references to “treating” or “treatment” include prevention as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms. As used herein, “treating” and like terms may specifically include reducing the severity and/or frequency of HSV induced symptoms, eliminating HSV induced symptoms and/or the underlying cause of said symptoms, reducing the frequency or likelihood of HSV induced symptoms and/or their underlying cause, delaying, preventing and/or slowing the progression of HSV induced conditions, and improving or remediating damage caused, directly or indirectly, by HSV infections. The term “preventing,” as used herein with respect to an HSV infection or HSV-related disorder, refers to reducing the likelihood of HSV infection. In one embodiment, “treating” or “treatment” of a state, disorder or condition means inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms. Conveniently, “treating” and like terms mean reducing the severity and/or frequency of HSV induced symptoms, eliminating HSV induced symptoms and/or the underlying cause of said symptoms, reducing the frequency or likelihood of HSV induced symptoms and/or their underlying cause, delaying, preventing and/or slowing the progression of HSV induced conditions, and/or improving or remediating damage caused, directly or indirectly, by HSV infections.

A “therapeutically effective amount” or “therapeutically effective dose” means the amount of a compound that, when administered to a patient or subject for treating a disease, is sufficient to effect such treatment for the disease. As used herein, the phrase “therapeutically effective dose” or “therapeutically effective amount” may specifically refer to the amount of Compound 1 dosed to a patient or subject using a pharmaceutical composition, as described herein, which is effective to achieve a particular biological or therapeutic result such as, but not limited to, biological or therapeutic results disclosed, described, or exemplified herein. The therapeutically effective dose may vary according to factors such as the disease state, age, sex, and weight of the individual, and the ability of the composition to cause a desired response in a subject. Such results include, but are not limited to, the reduction, remission, and/or regression of conditions caused by, or associated with, HSV or prevention of the development of conditions caused by, or associated with, HSV, as determined by any means suitable in the art.

When values are expressed as approximations, by use of the antecedent “about,” it will be understood that the particular value forms another embodiment. Further, the term “about” refers to a ±10% variation from the nominal value unless otherwise indicated or inferred.

The term “about” when used in reference to numerical ranges, cut-offs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

At various places in the present specification, values are disclosed in groups or in ranges. It is specifically intended that the description include all individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

As used herein, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

The use of any and all examples, or exemplary language herein, for example, “such as,” “including,” or “for example,” is intended merely to illustrate better the present teachings and does not pose a limitation on the scope of the invention unless claimed. No language in the specification should be construed as indicating any non-claimed element as essential to the practice of the present teachings.

### Helicase-Primase Inhibitor, Compound 1

The expression “helicase-primase inhibitor” within the context of the present invention denotes a compound or agent able to reduce viral replication by inhibiting the viral complex consisting of DNA helicase, DNA primase, and cofactor subunits. The helicase-primase complex is used by herpes viruses, therefore the helicase-primase inhibitor will have antiviral activity against one or more herpes viruses, such as one or more of Herpes Simplex Virus subtype type 1 and 2 (HSV-1, HSV-2), Varicella Zoster Virus (VZV), Epstein-Barr virus (EBV), Cytomegalovirus (CMV), and Human Herpes Viruses 6-8 (HHV 6-8).

The helicase-primase inhibitor has antiviral activity against HSV-1 and/or HSV-2.

The helicase-primase inhibitor is Compound 1 represented by:

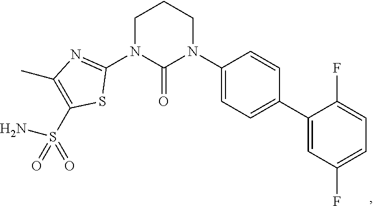 [SMILES: Cc1nc(N2CCCN(c3ccc(-c4cc(F)ccc4F)cc3)C2=O)sc1S(N)(=O)=O]

or a pharmaceutically acceptable salt thereof.

The compound shown above is 2-(3-(2′,5′-difluoro-[1,1′-biphenyl]-4-yl)-2-oxotetrahydropyrimidin-1(2H)-yl)-4-methylthiazole-5-sulfonamide (also referred to herein as ‘Compound 1’) and is a potent helicase-primase inhibitor. The compound is disclosed as Example 22 in WO2024/049760A1 (PCT Application No. PCT/US2023/031285).

Compound 1 has an in-vitro EC₅₀ value of approximately 0.019 μM against HSV-1 and 0.011 μM against HSV-2. The in-vitro EC₅₀ value against HSV-1 and/or HSV-2 can be determined in accordance with methods known to the skilled person, such as those disclosed in Field et al. (2013, Antiviral Res. 100, p. 297-299). The in-vitro EC₅₀ value can also be determined in accordance with the assays described in the Examples section of the present application.

Compound 1 has been found to have a predicted human biological terminal half-life of over 180 hours (see Example 3 and 3A).

Compound 1 shows no or low levels of carbonic anhydrase inhibition, such as inhibition of carbonic anhydrase I and/or carbonic anhydrase II. Carbonic anhydrase inhibition can be measured using a carbonic anhydrase I assay as described in Katritzky et al. (J. Med. Chem. 1987, 30:2058) and a carbonic anhydrase I assay as described in Iyer et al. (J. Biomol. Screen 2006, 11:782).

Compound 1 is poorly soluble and has a solubility in water at ~ pH 7.0 (measured at room temperature) of less than 5 μg/ml.

Compound 1 of the present invention comprises both a parent compound and any pharmaceutically acceptable salt of the parent compound. It is to be understood that Compound 1 may exhibit polymorphism, and that the invention encompasses all such forms (including anhydrous/non-solvated forms, solvates and hydrates).

### Pharmaceutical Compositions of the Invention

In a first aspect, the present invention provides an oral pharmaceutical composition comprising a solid dispersion, wherein the solid dispersion comprises a compound (Compound 1) represented by:

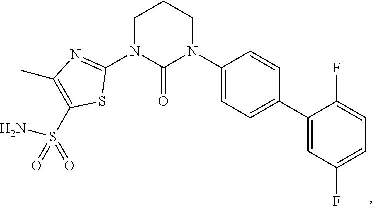 [SMILES: Cc1nc(N2CCCN(c3ccc(-c4cc(F)ccc4F)cc3)C2=O)sc1S(N)(=O)=O]

or a pharmaceutically acceptable salt thereof, and at least one matrix polymer.

Compositions of the invention provide high oral bioavailability and/or are long-acting. Despite Compound 1 having poor bioavailability, it has been found that when formulated in pharmaceutical compositions of the present invention, which comprise a solid dispersion comprising Compound 1 or a pharmaceutically acceptable salt thereof and at least one matrix polymer, immediate release of drug with significantly improved solubility and dissolution rate can be achieved. Furthermore, drug can be maintained in solution for a significantly prolonged period following administration to provide a good level of prolonged exposure when dosed.

In certain embodiments, pharmaceutical compositions of the present invention comprise a solid dispersion comprising Compound 1 or a pharmaceutically acceptable salt thereof and at least one matrix polymer, wherein the Compound 1 or a pharmaceutically acceptable salt thereof is stabilised within the solid dispersion, for example in a substantially amorphous (or fully amorphous) form. Such compositions have been found to provide rapid release of drug with significant improvements in solubility and dissolution rate. Furthermore, drug can be maintained in solution for a significantly prolonged period following administration to provide a good level of prolonged exposure when dosed. As used herein, the expression “stabilised within the solid dispersion in a substantially amorphous form” refers to the Compound 1 or a pharmaceutically acceptable salt thereof being maintained in the solid dispersion in a substantially amorphous form, e.g. by the aid of formulation approaches and excipients, such as the at least one matrix polymer, in order to provide beneficial solubility and dissolution rate properties upon administration of the pharmaceutical composition. Conveniently, Compound 1 or a pharmaceutically acceptable salt thereof is stabilised within the pharmaceutical composition in amorphous form.

In an embodiment, the present invention provides an oral pharmaceutical composition comprising a solid dispersion, wherein the solid dispersion comprises:
a. Compound 1 or a pharmaceutically acceptable salt thereof, and
b. at least one matrix polymer;
wherein the composition optionally contains one or more pharmaceutically acceptable excipients.

In an embodiment, the solid dispersion is a substantially amorphous solid dispersion. Conveniently, Compound 1 (or a pharmaceutically acceptable salt thereof) is present at less than about 15% (such as less than about 10%, less than about 5%, less than about 2%, or less than about 1%) crystalline form within the solid dispersion or composition. The skilled person will understand that various suitable methods exist to determine the crystallinity levels of a compound in solid dispersion compositions including Raman mapping, hot stage microscopy, atomic force microscopy, PLM, High Resolution PXRD, Solid State NMR, TAM (Thermal Activity Monitor), and FT-Raman. Where crystalline drug is present it may exist as crystalline domains or nanocrystalline domains, within a dispersion, e.g. within an amorphous dispersion.

In an embodiment, the solid dispersion is an amorphous solid dispersion. An amorphous dispersion may refer to a dispersion of amorphous compound in a matrix polymer, where the drug is in the molecularly dispersed state—also referred to sometimes as a glass solution. In such a glass solution, the drug and matrix polymer are molecularly dispersed with each other in a single homogeneous phase, and a differential scanning calorimetry (DSC) shows a single glass transition temperature (T_{g}) peak.

Two-phase blends, also known as solid glassy suspensions, contain a compound in a partially miscible state with the polymer and are more prone to undergo phase separation during storage. A solid crystalline suspension can contain a polymer in an amorphous phase while the compound is in a crystalline phase. A DSC of such a suspension shows one T_{g} peak for the polymer and one melting peak for the compound, which indicates no miscibility between the compound and the polymer.

In an embodiment, the solid dispersion has a single glass transition temperature (T_{g}).

In an embodiment, the solid dispersion comprises about 5 wt % to about 60 wt % of Compound 1, or a pharmaceutically acceptable salt thereof. In a convenient embodiment, the solid dispersion comprises about 30 wt % to about 60 wt % of Compound 1, or a pharmaceutically acceptable salt thereof. In a convenient embodiment, the solid dispersion comprises about 10 wt % to about 50 wt % of Compound 1, or a pharmaceutically acceptable salt thereof. Conveniently, the solid dispersion comprises about 10 wt % to about 30 wt % of Compound 1, or a pharmaceutically acceptable salt thereof. Conveniently, the solid dispersion comprises about 15 wt % or about 20 wt % of Compound 1, or a pharmaceutically acceptable salt thereof.

In an embodiment, the solid dispersion comprises about 30 wt % to about 95 wt % of the at least one matrix polymer. In a convenient embodiment, the solid dispersion comprises about 40 wt % to about 90 wt %, such as about 50 wt % to about 90 wt %, about 60 wt % to about 90 wt %, about 70 wt % to about 90 wt %, about 75 wt % to about 85 wt % of the at least one matrix polymer. In a convenient embodiment, the solid dispersion comprises about 60 wt % to about 90 wt %, about 70 wt % to about 90 wt %, about 80 wt % of the at least one matrix polymer.

In an embodiment, the solid dispersion comprises a wt:wt ratio of Compound 1, or a pharmaceutically acceptable salt thereof, to the at least one matrix polymer of between 1:10 and 1:1. Conveniently, the solid dispersion comprises a wt:wt ratio of Compound 1, or a pharmaceutically acceptable salt thereof, to the at least one matrix polymer of between 1:10 and 1:2 or between 1:10 and 1:3. Conveniently, the solid dispersion comprises a wt:wt ratio of Compound 1, or a pharmaceutically acceptable salt thereof, to the at least one matrix polymer is about 1:4.

In an embodiment, the at least one matrix polymer is one matrix polymer. In an embodiment, the at least one matrix polymer is two matrix polymers. In a convenient embodiment, there is only one matrix polymer present within the solid dispersion.

In an embodiment, the at least one matrix polymer is a water insoluble, or water soluble, ionic or neutral polymer. In an embodiment, the at least one matrix polymer is a water insoluble neutral polymer. Conveniently, the at least one matrix polymer is a water-soluble neutral polymer. Conveniently, the at least one matrix polymer is a water-soluble ionic polymer. Conveniently, the at least one matrix polymer is a pH-sensitive polymer. A pH-sensitive polymer displays aqueous solubility dependent on the pH of the aqueous medium. For example, a pH-sensitive polymer may contain acidic groups which are ionised, or partially ionised, at physiological pH, so that the polymer dissolves at this pH. In an embodiment, the pH-sensitive polymer dissolves in aqueous media at above about pH 5.5, such as above about pH 6.0, above about pH 6.5, or above about pH 7.0. In an embodiment, the pH-sensitive polymer does not dissolve in aqueous media at below about pH 5.5, such as below about pH 6.0, or below about pH 6.5. In an embodiment, the matrix polymer does not dissolve in aqueous media at below about pH 6.0.

In an embodiment, the at least one matrix polymer is an ionic polymer or a pH-sensitive polymer, optionally wherein the pH-sensitive polymer dissolves in aqueous media at above about pH 5.5;

In an embodiment, the at least one matrix polymer is selected from a povidone polymer, a copovidone polymer, a methacrylate polymer, a polymethacrylate-based copolymer, poly(vinyl caprolactam-co-vinyl acetate-co-ethylene glycol), hydroxypropyl methylcellulose, hydroxypropyl methylcellulose acetate succinate and hydroxypropyl methylcellulose phthalate.

In an embodiment, the povidone polymer is Plasdone™, such as Plasdone™ K-12, Plasdone™ K-17, Plasdone™ K-25, Plasdone™ K-29/32 or Plasdone™ K-90. In an embodiment, the povidone polymer is Kollidon® 30. In an embodiment, the povidone polymer is crospovidone, such as Polyplasdone™.

In an embodiment, the copovidone polymer is PVP-VA64, Kollidon® VA64, Plasdone™ S-630 or Plasdone™ S-630 Ultra.

In an embodiment, the methacrylate polymer is a methacrylic acid copolymer selected from the group consisting of: a methacrylic acid copolymer, methacrylic acid—methacrylate copolymer, methacrylic acid—ethyl acrylate copolymer, ammonium methacrylate copolymer, and aminoalkyl methacrylate copolymer. In certain embodiments, the methacrylic acid copolymer is EUDRAGIT® L 100 or EUDRAGIT® L 12,5 (also referred to as, or conforms with: “Methacrylic Acid Copolymer, Type A;” “Methacrylic Acid—Methyl Methacrylate Copolymer (1:1);” “Methacrylic Acid Copolymer L;” “DMF 1242” or “PR-MF 6918”); EUDRAGIT® S 100 and EUDRAGIT® S 12,5 (also referred to as, or conforms with: “Methacrylic Acid Copolymer, Type B;” “Methacrylic Acid—Methyl Methacrylate Copolymer (1:2);” “Methacrylic Acid Copolymer S;” “DMF 1242” or “PR-MF 6918”); EUDRAGIT® L 100-55 (also referred to as, or conforms with: “Methacrylic Acid Copolymer, Type C;” “Methacrylic Acid-Ethyl Acrylate Copolymer (1:1) Type A;” “Dried Methacrylic Acid Copolymer LD;” or “DMF 2584”); EUDRAGIT® L 30 D-55 (also referred to as, or conforms with: “Methacrylic Acid Copolymer Dispersion;” “Methacrylic Acid—Ethyl Acrylate Copolymer (1:1) Dispersion 30 Percent;” “Methacrylic Acid Copolymer LD;” JPE DMF 2584; PR-MF 8216); EUDRAGIT® FS 30 D (also referred to as DMF 13941 or DMF 2006-176); EUDRAGIT® RL 100 (also referred to as, or conforms with: “Ammonio Methacrylate Copolymer, Type A;” “Ammonio Methacrylate Copolymer (Type A);” “Aminoalkyl Methacrylate Copolymer RS;” “DMF 1242” or “PR-MF 6918”); EUDRAGIT® RL PO (also referred to as, or conforms with: “Ammonio Methacrylate Copolymer, Type A;” “Ammonio Methacrylate Copolymer (Type A);” “Aminoalkyl Methacrylate Copolymer RS;” “DMF 1242”); EUDRAGIT® RL 12,5 (also referred to as, or conforms with “Ammonio Methacrylate Copolymer, Type A;” “Ammonio Methacrylate Copolymer (Type A);” “DMF 1242” or “PR-MF 6918”); EUDRAGIT® L 100-55 (also referred to as, or conforms with: “Methacrylic Acid Copolymer, Type C;” “Methacrylic Acid—Ethyl Acrylate Copolymer (1:1) Type A;” “Dried Methacrylic Acid Copolymer LD;” “DMF 2584”); EUDRAGIT® L 30 D-55 (also referred to as, or conforms with: “Methacrylic Acid Copolymer Dispersion” NF “Methacrylic Acid—Ethyl Acrylate Copolymer (1:1) Dispersion 30 Percent;” “Methacrylic Acid Copolymer LD;” “DMF 2584” or “PR-MF 8216”); EUDRAGIT® FS 30 D (also referred to as, or conforms with: “DMF 13941” or “DMF 2006-176”); EUDRAGIT® RL 100 (also referred to as, or conforms with: “Ammonio Methacrylate Copolymer, Type A;” “Ammonio Methacrylate Copolymer (Type A);” “Aminoalkyl Methacrylate Copolymer RS;” “DMF 1242;” or “PR-MF 6918”); EUDRAGIT® RL PO (also referred to as, or conforms with: “Ammonio Methacrylate Copolymer, Type A;” “Ammonio Methacrylate Copolymer (Type A);” “Aminoalkyl Methacrylate Copolymer RS;” or “DMF 1242”); EUDRAGIT® RL 12,5 (also referred to as, or conforms with: polymer conforms to “Ammonio Methacrylate Copolymer, Type A;” “Ammonio Methacrylate Copolymer (Type A);” “DMF 1242” or “PR-MF 6918”); EUDRAGIT® RL 30 D (also referred to as, or conforms with: “Ammonio Methacrylate Copolymer Dispersion, Type A;” “Ammonio Methacrylate Copolymer (Type A);” or “DMF 1242”); EUDRAGIT® RS 100 (also referred to as, or conforms with: “Ammonio Methacrylate Copolymer, Type B;” NF “Ammonio Methacrylate Copolymer (Type B);” “Aminoalkyl Methacrylate Copolymer RS;” “DMF 1242” or “PR-MF 6918”); EUDRAGIT® RS PO (also referred to as, or conforms with: “Ammonio Methacrylate Copolymer, Type B;” “Ammonio Methacrylate Copolymer (Type B);” “Aminoalkyl Methacrylate Copolymer RS;” or “DMF 1242”); EUDRAGIT® RS 12,5 (also referred to as, or conforms with: “Ammonio Methacrylate Copolymer, Type B;” NF polymer conforms to “Ammonio Methacrylate Copolymer (Type B);” “DMF 1242” or “PR-MF 6918”); EUDRAGIT® RS 30 D (also referred to as, or conforms with: “Ammonio Methacrylate Copolymer Dispersion, Type B;” NF polymer conforms to “Ammonio Methacrylate Copolymer (Type B);” or “DMF 1242”); EUDRAGIT® E 100 (also referred to as, or conforms with: “Amino Methacrylate Copolymer;” NF “Basic Butylated Methacrylate Copolymer;” “Aminoalkyl Methacrylate Copolymer E;” “DMF 1242” or “PR-MF 6918”); EUDRAGIT® E PO (also referred to as, or conforms with: “Basic Butylated Methacrylate Copolymer;” “Aminoalkyl Methacrylate Copolymer E;” “Amino Methacrylate Copolymer;” “DMF 1242”); EUDRAGIT® E 12,5 (also referred to as, or conforms with: “Amino Methacrylate Copolymer;” “Basic Butylated Methacrylate Copolymer;” “DMF 1242” or “PR-MF 6918”); EUDRAGIT® NE 30 D (also referred to as, or conforms with: “Ethyl Acrylate and Methyl Methacrylate Copolymer Dispersion;” “Polyacrylate Dispersion 30 Percent;” (“Poly(ethylacrylat-methylmethacrylat)-Dispersion 30%”); “Ethyl Acrylate Methyl Methacrylate Copolymer Dispersion;” “DMF 2822” or “PR-MF 6918”); EUDRAGIT® NE 40 D (also referred to as, or conforms with: DMF 2822); EUDRAGIT® NM 30 D (also referred to as “Polyacrylate Dispersion 30 Percent;” “(Poly(ethylacrylat-methylmethacrylat)-Dispersion 30%);” or “DMF 2822”; PLASTOID® B (also referred to as, or conforms with: “DMF 12102”), or the like.

In certain embodiments, the methacrylate polymer is a poly(methacrylic acid-co-methyl methacrylate).

In certain embodiments, the methacrylate polymer is EUDRAGIT® L100, EUDRAGIT® L100-55, or an equivalent thereof.

In certain embodiments, the methacrylate polymer is an anionic 1:1 methacrylic acid-methyl methacrylate copolymer with CAS number 25086-15-1.

In certain embodiments, the methacrylate polymer is an anionic 1:1 methacrylic acid-methyl methacrylate copolymer which dissolves in water above pH 6, having a weight average molecular mass of approximately 125,000 g/mol.

In certain embodiments, the methacrylate polymer is an anionic 1:1 methacrylic acid-methyl methacrylate copolymer, CAS number 25086-15-1, which dissolves in water above pH 6, having a weight average molecular mass of approximately 125,000 g/mol.

In an embodiment, poly(vinyl caprolactam-co-vinyl acetate-co-ethylene glycol) is Soluplus®.

In an embodiment, the hydroxypropyl methylcellulose (HPMC) has about 29% methoxyl groups and about 8.5% hydroxypropyl groups. In an embodiment, the hydroxypropyl methylcellulose is HPMC E5LV, such as METHOCEL™ E5LV.

In a convenient embodiment, the at least one matrix polymer is selected from a copovidone polymer, poly(vinyl caprolactam-co-vinyl acetate-co-ethylene glycol), hydroxypropyl methylcellulose acetate succinate and hydroxypropyl methylcellulose phthalate.

In a convenient embodiment, the at least one matrix polymer is selected from hydroxypropyl methylcellulose acetate succinate, EUDRAGIT® L 100, EUDRAGIT® E 100, PVP-VA64, HPMC E3 and Soluplus®. In a convenient embodiment, the at least one matrix polymer is selected from hydroxypropyl methylcellulose acetate succinate, EUDRAGIT® L 100, PVP-VA64, HPMC E3 and Soluplus®. Conveniently, the at least one matrix polymer is selected from hydroxypropyl methylcellulose acetate succinate and Soluplus®.

In a convenient embodiment, the at least one matrix polymer is selected from hydroxypropyl methylcellulose acetate succinate and hydroxypropyl methylcellulose phthalate.

Certain matrix polymers (e.g. HPMCAS) have been found to be particularly advantageous in terms of providing solid dispersions. In a convenient embodiment, the at least one matrix polymer is hydroxypropyl methylcellulose acetate succinate (HPMCAS).

In a convenient embodiment, the hydroxypropyl methylcellulose acetate succinate dissolves in aqueous media at above about pH 5.5, about pH 6.0, or above about pH 6.5. In an embodiment, the hydroxypropyl methylcellulose acetate succinate does not dissolve in aqueous media at below about pH 6.0, or below about pH 6.5. In a convenient embodiment, the hydroxypropyl methylcellulose acetate succinate does not dissolve in aqueous media at below about pH 5.5. In a convenient embodiment, the hydroxypropyl methylcellulose acetate succinate does not dissolve in aqueous media at below about pH 6.0.

In a convenient embodiment, the hydroxypropyl methylcellulose acetate succinate has either:
i. an acetyl content of 5 to 9% w/w and a succinoyl content of 14 to 18% w/w;
ii. an acetyl content of 7 to 11% w/w and a succinoyl content of 10 to 14% w/w; or
iii. an acetyl content of 10 to 14% w/w and a succinoyl content of 4 to 8% w/w.

In a more convenient embodiment, the hydroxypropyl methylcellulose acetate succinate has an acetyl content of 7 to 11% w/w and a succinoyl content of 10 to 14% w/w.

In a more convenient embodiment, the hydroxypropyl methylcellulose acetate succinate has an acetyl content of 10 to 14% w/w and a succinoyl content of 4 to 8% w/w.

In an embodiment, the at least one matrix polymer is HMPCAS-L, HPMCAS-M or HPMCAS-H. Conveniently, the at least one matrix polymer is HPMCAS-M. Conveniently, the at least one matrix polymer is HPMCAS-L. More conveniently, the at least one matrix polymer is HPMCAS-H.

In an embodiment, the hydroxypropyl methylcellulose acetate succinate has a weight-average molecular weight of about 15,000 to 25,000, such as about 17,000 to 20,000.

The solid dispersions of the present invention have good stability. Therefore, in an embodiment, the solid dispersion is stable for at least four weeks, such as for at least eight, or twelve, weeks. In this context, ‘stable’ refers to physical and/or chemical stability of Compound 1 or a pharmaceutically acceptable salt thereof within the solid dispersion. Evidence of the dissolution profile or pharmacokinetics of the solid dispersion being maintained over the storage period referred to can serve as an indicator of stability.

Physical stability relates to maintenance of Compound 1 or a pharmaceutically acceptable salt thereof in the same physical form; when the solid dispersion is a substantially amorphous solid dispersion, the amount of crystalline Compound 1 or a pharmaceutically acceptable salt thereof does not increase significantly over the storage period referred to; when the solid dispersion is an amorphous solid dispersion, no (or a very low level of) crystallisation of Compound 1 or a pharmaceutically acceptable salt thereof is observed to occur over the storage period referred to. Therefore, in an embodiment, the solid dispersion does not show any Compound 1 or a pharmaceutically acceptable salt thereof crystalline peaks by XRPD, when stored for at least four weeks, such as for at least eight, or twelve, weeks, such as for at least twenty-four, or thirty-six weeks. In an embodiment, the solid dispersion does not show any crystalline peaks by XRPD, when stored for at least four weeks, such as for at least eight, or twelve, weeks at 25° C. and 60% relative humidity (RH). In an embodiment, the solid dispersion does not show any crystalline peaks by XRPD, when stored for at least twenty-four, or thirty-six, weeks at 25° C. and 60% RH. In an embodiment, the solid dispersion does not show any crystalline peaks by XRPD, when stored for at least four weeks, such as for at least eight, or twelve, weeks at 40° C. and 75% RH. In an embodiment, the solid dispersion does not show any crystalline peaks by XRPD, when stored for at least twenty-four, or thirty-six, weeks at 40° C. and 75% RH.

Chemical stability relates to low levels of impurities being formed over the storage period referred to. Typically, these are impurities related to Compound 1 or a pharmaceutically acceptable salt thereof. In an embodiment, the solid dispersion has total impurities by HPLC of less than 2.5%, such as less than 2.0%, less than 1.5%, less than 1.0%, or less than 0.5%, when stored for at least four weeks, such as for at least eight, or twelve, weeks at 25° C. and 60% relative humidity (RH).

The moisture content of the solid dispersion may have an influence on the dissolution profile or pharmacokinetics of the solid dispersion. The water content of the solid dispersion may be determined by various methods apparent to a person of skill in the art, such as Karl Fischer titration. In an embodiment, the solid dispersion has a water content of less than 5 wt %, such as less than 4 wt %, less than 3 wt %, less than 2 wt %, or less than 1.5 wt %.

The solid dispersions according to the present invention may be produced by techniques such as spray drying, hot melt extrusion or solvent granulation. In a convenient embodiment, the solid dispersion is a spray-dried solid dispersion.

In order to facilitate the formation of the solid dispersion, or to improve the stability of the solid dispersion, additional excipients may also be present in the solid dispersion. In an embodiment, the solid dispersion further comprises one or more excipients selected from the group consisting of a surfactant (such as sodium lauryl sulfate, polysorbates, and sorbitan esters), a diluent, a binder, an adsorbent (such as colloidal silica), a lubricant, a disintegrant, a drying agent, a pH modifier, a salt former, a complexing agent and a glidant.

In an embodiment, the solid dispersion contains no plasticizer. In an embodiment, the solid dispersion comprises Compound 1, or a pharmaceutically acceptable salt thereof, and at least one matrix polymer, but does not contain any additional excipients.

In an embodiment, the solid dispersion has a particle size distribution comprising 90% (Dv(90)) of the dispersion having a size of 250 μm or smaller, such as 150 μm, 75 μm, 50 μm, 35 μm or 20 μm, or smaller. The skilled person will be aware of suitable techniques, such as laser diffraction as described in the Examples section, that can be used to determine particle size and particle size distribution of solid dispersion material.

The present disclosure provides pharmaceutical compositions comprising a solid dispersion of Compound 1 or a pharmaceutically acceptable salt thereof with at least one matrix polymer, as discussed herein. In general, the pharmaceutical compositions can be formed by combining a solid dispersion of the disclosure with at least one excipient. The resulting pharmaceutical composition then can be formed into a unit dosage form. Therefore, there is provided a pharmaceutical composition comprising a solid dispersion as described herein, and further comprising one or more pharmaceutically acceptable excipients.

In an embodiment, the pharmaceutical composition comprises a surfactant. In an embodiment, the surfactant is a non-ionic surfactant. In an embodiment, the surfactant is an ionic surfactant. In an embodiment, the surfactant is selected from benzylalkonium chloride, benzethonium chloride, cetylpyridinium chloride, poloxamer 188, poloxamer 407, polyoxyl stearate, polysorbates, sodium lauryl sulfate, sorbitan monooleate, sorbitan monopalmitate, sorbitan monostearate, and Vitamin E TPGS. Conveniently, the surfactant is sodium lauryl sulfate. Conveniently, the surfactant is present at 0.25-2.0 wt %, such as about 1 wt %, of the pharmaceutical composition.

In an embodiment, the pharmaceutical composition further comprises a crystallization inhibitor. The crystallization inhibitor may delay or prevent nucleation of crystalline Compound 1 or a pharmaceutically acceptable salt thereof and it may function to stabilise the amorphous state of the solid dispersion and help maintain Compound 1 in solution.

The crystallization inhibitor may be (i) a surfactant as described above; (ii) a cellulose ether, such as hydroxypropyl methyl cellulose (HPMC), hydroxypropyl cellulose (HPC), hydroxyethyl methyl cellulose, hydroxyethyl cellulose, and hydroxyethyl ethyl cellulose; or (iii) a copovidone polymer, such as PVP-VA64. In an embodiment, the crystallization inhibitor is hydroxypropyl methylcellulose.

In an embodiment, the pharmaceutical composition further comprises one or more pharmaceutically acceptable excipients selected from a filler, a diluent, a binder, a disintegrant, a lubricant and a glidant.

Examples of pharmaceutically acceptable fillers or diluents include lactose, sugar, maize-starch, microcrystalline cellulose, mannitol, calcium phosphate, sorbitol and glycine. In an embodiment, the filler or diluent is selected from lactose, microcrystalline cellulose and mannitol; conveniently microcrystalline cellulose and mannitol. Conveniently, each filler or diluent is present at 5-30 wt % of the pharmaceutical composition, conveniently at 5-15 wt % of the pharmaceutical composition.

Examples of pharmaceutically acceptable binders include hydroxy propyl cellulose, hypromellose, povidone, starch, methylcellulose, gelatin, pregelatinized starch, and xanthan gum.

Examples of pharmaceutically acceptable disintegrants include crospovidone, croscarmellose, sodium starch glycolate and low substituted hydroxypropyl cellulose; conveniently the disintegrant is croscarmellose sodium. Conveniently, the disintegrant is present at 1.0-5.0 wt %, such as 2.0-4.0 wt %, of the pharmaceutical composition.

Examples of pharmaceutically acceptable lubricants include magnesium stearate, calcium stearate, hydrogenated vegetable oil, stearic acid, sodium stearyl fumarate, mineral oil, hydrogenated vegetable oil and polyethylene glycol; conveniently the lubricant is magnesium stearate. Conveniently, the lubricant is present at 0.5-2.0 wt %, such as 1.0-2.0 wt %, of the pharmaceutical composition.

Examples of pharmaceutically acceptable glidants include colloidal silicon dioxide, ascorbyl palmitate, calcium palmitate, starch and talc; conveniently the glidant is colloidal silicon dioxide. Conveniently, the glidant is present at 0.5-2.0 wt %, such as 1.0-2.0 wt %, of the pharmaceutical composition.

In an embodiment, the pharmaceutical composition is in a unit dosage form comprising about 1 mg to about 500 mg of Compound 1 or a pharmaceutically acceptable salt thereof.

In an embodiment, the pharmaceutical composition is in a unit dosage form suitable for oral administration. In an embodiment, the pharmaceutical composition is in a unit dosage form selected from the group consisting of a granule, a pellet, a tablet, a particle, a capsule, a suspension and a mini-tablet.

### Tablets

Conveniently, the pharmaceutical compositions of the present invention may be formulated as tablets for oral administration.

Tablets may include pills, caplets, mini-tablets, micro-tablets and/or orally disintegrating tablets. Tablets of the present invention can be any shape or size. In an embodiment, the pharmaceutical composition is a tablet with a total weight of 50-1000 mg, such as less than 1000 mg, such as less than 900 mg, 50-150 mg, 100-300 mg, or 750-950 mg. In an embodiment, the pharmaceutical composition is a tablet with a total weight of 250-500 mg, conveniently 300-500 mg, such as about 400 mg.

In an embodiment, the tablet comprises 50-70 wt % of the solid dispersion according to the present invention. Conveniently, the tablet comprises 55-65 wt % of the solid dispersion according to the present invention.

It is to be understood that in the present context the “wt %” or “% by weight” values specified herein for tablet dosage forms refer to the percentage by weight of an ingredient in the core tablet, thus excluding any exterior coatings or films.

In an embodiment, the tablet disintegrates in less than 15 minutes when tested in water at 37° C. according to USP disintegration test protocol. In an embodiment, the tablet disintegrates in less than 10 minutes, such as less than 7 minutes, or less than 5 minutes, when tested in 0.01N hydrochloric acid (SGF) at 37° C. according to USP disintegration test protocol.

Tablet compositions according to the present invention comprise a solid dispersion and optionally one or more pharmaceutically acceptable excipients.

In an embodiment, the one or more pharmaceutically acceptable excipients are selected from a filler, a diluent, a binder, a surfactant, a disintegrant, a lubricant, a glidant, and a crystallization inhibitor. Conveniently, the one or more pharmaceutically acceptable excipients are selected from a filler, a diluent, a disintegrant, a lubricant and a glidant.

In an embodiment, the tablet pharmaceutical composition comprises a surfactant. In an embodiment, the surfactant is a non-ionic surfactant. In an embodiment, the surfactant is an ionic surfactant. In an embodiment, the surfactant is selected from benzylalkonium chloride, benzethonium chloride, cetylpyridinium chloride, poloxamer 188, poloxamer 407, polyoxyl stearate, polysorbates, sodium lauryl sulfate, sorbitan monooleate, sorbitan monopalmitate, sorbitan monostearate, and Vitamin E TPGS. Conveniently, the surfactant is sodium lauryl sulfate. Conveniently, the surfactant is present at 0.25-2.0 wt %, such as about 1 wt %, of the tablet.

In an embodiment, the tablet pharmaceutical composition further comprises at least one pharmaceutically acceptable filler or diluent. In an embodiment, the filler or diluent is selected from lactose, sugar, maize-starch, microcrystalline cellulose, mannitol, calcium phosphate, sorbitol and glycine. In an embodiment, the filler or diluent is selected from lactose, microcrystalline cellulose and mannitol, conveniently microcrystalline cellulose and mannitol. Conveniently, each filler or diluent is present at 5-30 wt % of the tablet, conveniently at 5-15 wt % of the tablet.

In an embodiment, the tablet pharmaceutical composition further comprises a pharmaceutically acceptable disintegrant. Examples of pharmaceutically acceptable disintegrants include crospovidone, croscarmellose, sodium starch glycolate and low substituted hydroxypropyl cellulose; conveniently the disintegrant is croscarmellose sodium. Conveniently, the disintegrant is present at 1.0-5.0 wt %, such as 2.0-4.0 wt %, of the tablet.

In an embodiment, the tablet pharmaceutical composition further comprises a pharmaceutically acceptable lubricant. Examples of pharmaceutically acceptable lubricants include magnesium stearate, calcium stearate, hydrogenated vegetable oil, stearic acid, sodium stearyl fumarate, mineral oil, hydrogenated vegetable oil and polyethylene glycol; conveniently the lubricant is magnesium stearate. Conveniently, the lubricant is present at 0.5-2.0 wt %, such as 1.0-2.0 wt %, of the tablet.

In an embodiment, the tablet pharmaceutical composition further comprises a pharmaceutically acceptable glidant. Examples of pharmaceutically acceptable glidants include colloidal silicon dioxide, ascorbyl palmitate, calcium palmitate, starch and talc; conveniently the glidant is colloidal silicon dioxide. Conveniently, the glidant is present at 0.5-2.0 wt %, such as 1.0-2.0 wt %, of the tablet.

When formed by compression, the tablet conveniently has a “strength” of at least 5 kiloponds (kp)/cm², and more preferably at least 7 kp/cm². Here, “strength” is the fracture force, also known as the tablet “hardness,” required to fracture a tablet formed from the materials, divided by the maximum cross-sectional area of the tablet normal to that force. The compression force required to achieve this strength will depend on various factors such as for example, the size of the tablet, but generally the strength will be greater than about 5 kp/cm². In a convenient embodiment, the tablets have a strength of about 10-25 kp/cm².

Optionally, the tablet may be coated by methods well known in the art. The coating may be a pH-independent coating (such as seal coatings or film coatings) or a pH-dependent coating (such as enteric coatings or modified release coatings).

### In Vitro Dissolution

In order to mimic oral administration, the solid dispersions and pharmaceutical compositions of the present invention may be tested in a two-stage bio-relevant dissolution method. The method comprises an initial (0-30 minutes) acidic phase conducted at pH 2 using simulated gastric fluid, followed by a subsequent (30-210 minutes) neutral phase conducted at pH 6.8 using FaSSIF media-0.1M pH 6.8 buffer comprising 2.24 mg/ml FaSSIF powder.

### Pharmacokinetics

As mentioned previously, it has been found that the compositions of the present invention are particularly advantageous at improving the bioavailability of Compound 1 or a pharmaceutically acceptable salt thereof. Compositions of the invention provide the benefit of both an immediate release of the drug to reach efficacious antiviral levels, followed by prolonged release of the drug to provide high plasma levels for prolonged periods.

Advantageously, certain compositions of the present invention may have high and/or equivalent, or superior, bioavailability to oral solutions. Therefore, in an embodiment, there is provided a pharmaceutical composition as described herein, wherein the composition has a bioavailability relative to an oral solution of greater than 50%, such as greater than 70%, greater than 80%, greater than 90%, or greater than 100%.

Advantageously, certain compositions of the present invention may exhibit low PK variability in plasma concentrations upon administration. Advantageously, certain compositions of the present invention may exhibit reduced PK variability when compared to oral solutions, even at high doses.

In certain embodiments, compositions of the present invention may exhibit low PK variability in plasma concentrations of Compound 1, upon oral administration to a subject despite variation in the gastric pH of the subject, such as variation from about pH 1 to pH 7.

It has also been surprisingly found that certain compositions of the present invention are able to provide higher plasma levels of Compound 1 for a prolonged period when administered in connection with the intake of food. As such, in some embodiments, compositions of the present invention are administered in connection with the intake of food.

### Particular Compositions of the Invention

In one embodiment, the present invention provides an oral pharmaceutical composition comprising a solid dispersion, wherein the solid dispersion comprises Compound 1, or a pharmaceutically acceptable salt thereof, and at least one matrix polymer, and wherein the solid dispersion comprises amorphous Compound 1 or a pharmaceutically acceptable salt thereof in an amorphous matrix polymer. Conveniently, Compound 1 is present in the solid dispersion in its neutral free form. More conveniently, the solid dispersion is a spray-dried solid dispersion.

In one embodiment, the present invention provides an oral pharmaceutical composition comprising a solid dispersion, wherein the solid dispersion comprises Compound 1, and at least one matrix polymer, wherein the solid dispersion is an amorphous solid dispersion, and wherein the solid dispersion comprises:
a) about 10 wt % to about 30 wt % of Compound 1; and
b) about 60 wt % to about 90 wt % or about 70 wt % to about 90 wt % of the at least one matrix polymer.

In one embodiment, the present invention provides an oral pharmaceutical composition comprising a solid dispersion, wherein the solid dispersion comprises Compound 1, and at least one matrix polymer, wherein the solid dispersion is an amorphous solid dispersion, and wherein the solid dispersion comprises:
a) about 30 wt % to about 60 wt % of Compound 1; and
b) about 40 wt % to about 70 wt % of the at least one matrix polymer.

In one embodiment, the present invention provides an oral pharmaceutical composition comprising a solid dispersion, wherein
a) the solid dispersion comprises Compound 1, and at least one matrix polymer;
b) the solid dispersion is an amorphous solid dispersion; and
c) the solid dispersion comprises a wt:wt ratio of Compound 1 to the at least one matrix polymer of between 1:10 and 1:2.

In one embodiment, the present invention provides an oral pharmaceutical composition comprising a solid dispersion, wherein the solid dispersion comprises Compound 1, or a pharmaceutically acceptable salt thereof, and at least one matrix polymer, wherein the solid dispersion is an amorphous solid dispersion, and wherein the at least one matrix polymer is a water soluble ionic or neutral polymer.

In one embodiment, the present invention provides an oral pharmaceutical composition comprising a solid dispersion, wherein the solid dispersion comprises Compound 1, or a pharmaceutically acceptable salt thereof, and at least one matrix polymer, wherein the solid dispersion is an amorphous solid dispersion, and wherein the at least one matrix polymer is:
i. an ionic polymer or a pH-sensitive polymer, optionally wherein the pH-sensitive polymer dissolves in aqueous media at above about pH 5.5;
ii. selected from a povidone polymer, a copovidone polymer, a methacrylate polymer, a polymethacrylate-based copolymer, poly(vinyl caprolactam-co-vinyl acetate-co-ethylene glycol), hydroxypropyl methylcellulose, hydroxypropyl methylcellulose acetate succinate and hydroxypropyl methylcellulose phthalate;
iii. selected from a copovidone polymer, poly(vinyl caprolactam-co-vinyl acetate-co-ethylene glycol), hydroxypropyl methylcellulose acetate succinate and hydroxypropyl methylcellulose phthalate; or
iv. selected from hydroxypropyl methylcellulose acetate succinate and hydroxypropyl methylcellulose phthalate.

In one embodiment, the present invention provides an oral pharmaceutical composition comprising a solid dispersion, wherein the solid dispersion comprises Compound 1, or a pharmaceutically acceptable salt thereof, and at least one matrix polymer, wherein the solid dispersion is an amorphous solid dispersion, and wherein the at least one matrix polymer is selected from hydroxypropyl methylcellulose acetate succinate, EUDRAGIT® L 100, PVP-VA64, HPMC E3 and Soluplus®. Conveniently, Compound 1 is present in the solid dispersion in its neutral free form. More conveniently, the solid dispersion is a spray-dried solid dispersion.

In one embodiment, the present invention provides an oral pharmaceutical composition comprising a solid dispersion, wherein the solid dispersion comprises Compound 1, and a matrix polymer, wherein the solid dispersion is an amorphous solid dispersion, and wherein the matrix polymer is selected from hydroxypropyl methylcellulose acetate succinate, EUDRAGIT® L 100, PVP-VA64, HPMC E3 and Soluplus®. Conveniently, the matrix polymer is selected from hydroxypropyl methylcellulose acetate succinate and Soluplus®.

In one embodiment, the present invention provides an oral pharmaceutical composition comprising a solid dispersion, wherein the solid dispersion comprises Compound 1, and a matrix polymer, wherein the solid dispersion is an amorphous solid dispersion, and wherein the matrix polymer is selected from HPMCAS-H and Soluplus®. Conveniently, the solid dispersion is a spray-dried solid dispersion.

In one embodiment, the present invention provides an oral pharmaceutical composition comprising a solid dispersion (such as a spray dried solid dispersion), wherein
a) the solid dispersion comprises Compound 1, and at least one matrix polymer;
b) the solid dispersion is an amorphous solid dispersion;
c) the solid dispersion comprises a wt:wt ratio of Compound 1 to the at least one matrix polymer of between 1:10 and 1:2; and
d) the at least one matrix polymer is hydroxypropyl methylcellulose acetate succinate.

In one embodiment, the present invention provides an oral pharmaceutical composition comprising a solid dispersion (such as a spray dried solid dispersion), wherein
a) the solid dispersion comprises Compound 1, and at least one matrix polymer;
b) the solid dispersion is an amorphous solid dispersion;
c) the solid dispersion comprises a wt:wt ratio of Compound 1 to the at least one matrix polymer of between 1:10 and 1:2; and
d) the at least one matrix polymer is Soluplus®.

In one embodiment, the present invention provides an oral pharmaceutical composition comprising a solid dispersion (such as a spray dried solid dispersion), wherein:
a) the solid dispersion comprises Compound 1, and a matrix polymer;
b) the solid dispersion is an amorphous solid dispersion;
c) the matrix polymer is a water insoluble or water soluble ionic or neutral polymer (conveniently the matrix polymer is selected from hydroxypropyl methylcellulose acetate succinate, EUDRAGIT® L 100, PVP-VA64, HPMC E3 and Soluplus®); and
d) the solid dispersion further comprises one or more excipients selected from the group comprising of a surfactant (such as sodium lauryl sulfate), a diluent (such as lactose, microcrystalline cellulose and/or mannitol), a binder, a lubricant (such as magnesium stearate), a disintegrant (such as croscarmellose sodium), a drying agent, a pH modifier, a salt former, a complexing agent and a glidant (such as colloidal silicon dioxide).

In one embodiment, the present invention provides an oral tablet pharmaceutical composition comprising a solid dispersion (such as a spray dried solid dispersion), wherein:
a) the solid dispersion comprises Compound 1, and at least one matrix polymer;
b) the solid dispersion is an amorphous solid dispersion;
c) the solid dispersion comprises a wt:wt ratio of Compound 1 to the at least one matrix polymer of between 1:10 and 1:2; and
d) the oral tablet pharmaceutical composition further comprises at least one or more pharmaceutically acceptable excipients selected from a filler, a diluent, a surfactant, a disintegrant, a lubricant and a glidant.

Conveniently, the at least one or more pharmaceutically acceptable excipients selected in the oral tablet pharmaceutical composition comprise:
a) a surfactant selected from benzylalkonium chloride, benzethonium chloride, cetylpyridinium chloride, poloxamer 188, poloxamer 407, polyoxyl stearate, polysorbates, sodium lauryl sulfate, sorbitan monooleate, sorbitan monopalmitate, sorbitan monostearate, and Vitamin E TPGS;
b) at least one pharmaceutically acceptable filler or diluent selected from lactose, sugar, maize-starch, microcrystalline cellulose, mannitol, calcium phosphate, sorbitol and glycine;
c) a pharmaceutically acceptable disintegrant selected from crospovidone, croscarmellose, sodium starch glycolate and low substituted hydroxypropyl cellulose;
d) a pharmaceutically acceptable lubricant selected from magnesium stearate, calcium stearate, hydrogenated vegetable oil, stearic acid, sodium stearyl fumarate, mineral oil, hydrogenated vegetable oil and polyethylene glycol; and
e) a pharmaceutically acceptable glidant selected from colloidal silicon dioxide, ascorbyl palmitate, calcium palmitate, starch and talc.

### Preparation of the Compositions

In a second aspect, the present invention provides a method for forming a pharmaceutical composition according to the first aspect.

Compositions according to the present invention comprise a solid dispersion and optionally one or more pharmaceutically acceptable excipients. The solid dispersion can be formed by any known technique. It should be appreciated that solid dispersions of the present invention can be prepared by methods such as spray drying, melt extrusion, co-precipitation, solvent controlled co-precipitation, freeze drying, kneading technique, co-grinding, gel entrapment, electrospinning and/or spin-coating. In certain embodiments (such as spray drying), compound and at least one matrix polymer are dissolved in a solvent to form a mixture, and the solvent is evaporated to form a solid dispersion. In certain embodiments (such as hot melt extrusion), the solid dispersion is formed without the use of solvents.

In an embodiment of the second aspect, the method comprises the steps of:
i) combining Compound 1, or a pharmaceutically acceptable salt thereof, and the at least one matrix polymer in a solvent to form a mixture;
ii) drying the mixture to form a solid dispersion; and
iii) optionally, combining the solid dispersion with one or more pharmaceutically acceptable excipients selected from a filler, a diluent, a binder, a surfactant, a disintegrant, a lubricant, a glidant, and a crystallization inhibitor.

In an embodiment, the solvent in step i) is an organic solvent. In an embodiment, the solvent in step i) is a combination of water and an organic solvent.

Organic solvents can include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, 1,1,1-trichloroethane, and tetrahydrofuran. Supercritical carbon dioxide can also be used as a solvent, or supercritical carbon dioxide can be used with an organic co-solvent, such as acetone, methanol, ethanol, and/or acetonitrile. Preferred organic solvents are methanol, acetone, tetrahydrofuran, ethyl acetate, methylene chloride, and mixtures thereof. In an embodiment, the organic solvent is a water-miscible organic solvent, such as methanol, ethanol, n-propanol, iso-propanol, acetone or acetonitrile. In an embodiment, the organic solvent is acetone. In an embodiment, the solvent in step i) is a combination of water and a water-miscible organic solvent. In an embodiment, the solvent in step i) is a combination of water and acetone. In an embodiment, the solvent in step i) is a combination of water and acetone in a v/v ratio between 1:99 and 30:70, such as between 1:99 and 10:90, or about 5:95.

The at least one matrix polymer in step i) is selected from one or more of the polymers described herein for the compositions according to the first aspect of the invention. In an embodiment, the at least one matrix polymer is selected from hydroxypropyl methylcellulose acetate succinate and hydroxypropyl methylcellulose phthalate. In an embodiment, the at least one matrix polymer is hydroxypropyl methylcellulose acetate succinate.

In an embodiment, in step (i) about 1 to 60 wt % of Compound 1, or a pharmaceutically acceptable salt thereof, is combined with about 30 to 95 wt % of the at least one matrix polymer.

In an embodiment, the solids content of the spray solution is between 2 and 20% w/w.

In certain embodiments, a surfactant is added to the mixture in step i). For example, a surfactant such as sodium lauryl sulfate (SLS), polysorbates, or sorbitan esters can be added.

In certain embodiments, one or more further excipients are added to the mixture in step i). Suitable excipients may include crystallization inhibitors, glidants, disintegrants, pH modifiers, salt formers, or complexing agents. In an embodiment, the glidant is colloidal silicon dioxide.

After at least a portion of Compound 1 and the at least one matrix polymer have been dissolved, the solvent can be removed in step ii) by evaporation, or by mixing with a non-solvent. Exemplary processes are spray-drying, spray-coating (e.g., pan-coating and fluidized bed coating), and precipitation by rapid mixing of the compound and polymer mixture with carbon dioxide (CO₂), hexane, heptane, water of appropriate pH, or some other non-solvent.

Preferably, removal of the solvent in step ii) results in a solid dispersion that is substantially homogeneous. To achieve this end, it is generally desirable to rapidly remove the solvent from the solution such as in a process where the solution is atomized, and the compound and the dispersion polymer rapidly solidify.

In certain embodiments, solvent can be removed by spray-drying, e.g., a process that involves breaking up liquid mixtures into small droplets (atomization) and rapidly removing solvent from the mixture in a spray-drying apparatus where there is a strong driving force for evaporation of solvent from the droplets. Spray-drying processes and spray-drying equipment are described generally in Perry's Chemical Engineers' Handbook, pages 20-54 to 20-57 (Sixth Edition, 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition, 1985). The strong driving force for solvent evaporation is generally provided by maintaining the partial pressure of solvent in the spray-drying apparatus well below the vapor pressure of the solvent at the temperature of the drying droplets. This can be accomplished by (1) maintaining the pressure in the spray-drying apparatus at a partial pressure; or (2) mixing the liquid droplets with a warm drying gas; or (3) both (1) and (2). In addition, at least a portion of the heat required for evaporation of solvent can be provided by heating the spray solution.

In a convenient embodiment, in step (ii) the drying comprises spray drying.

The solvent-bearing feed can be spray-dried under a wide variety of conditions and yet still yield solid dispersions with acceptable properties. For example, various types of nozzles can be used to atomize the spray solution, thereby introducing the spray solution into the spray-dry chamber as a collection of small droplets. Essentially any type of nozzle can be used to spray the solution as long as the droplets that are formed are sufficiently small that they dry sufficiently (due to evaporation of solvent) such that they do not stick to or coat the spray-drying chamber wall.

Outlet air temperature is a parameter which may affect the product morphology like particle size, surface roughness, density, stickiness of particles, residual solvent or moisture levels, product yield, etc. In an embodiment, the outlet air temperature is 30 to 50° C., such as 40 to 50° C., or about 45° C.

The spray solution can be delivered to the spray nozzle(s) at a wide range of temperatures and flow rates. Generally, the spray solution temperature can range anywhere from just above the solvent's freezing point to about 20° C. above its ambient pressure boiling point (by pressurizing the solution) and in some cases even higher. Spray solution flow rates to the spray nozzle can vary over a wide range depending on the type of nozzle, spray-dryer size and spray-dry conditions such as the inlet temperature and flow rate of the drying gas. Generally, the energy for evaporation of solvent from the spray solution in a spray-drying process comes primarily from the drying gas.

The drying gas can, in principle, be essentially any gas, and can be an inert gas such as nitrogen, nitrogen-enriched air or argon. The drying gas is typically introduced into the drying chamber at a temperature between about 60° C. and about 300° C. and preferably between about 80° C. and about 240° C. Other drying gas temperatures could also be used in forming solid dispersions of the disclosure.

Generally, the solvent content of the solid dispersion as it leaves the spray-drying chamber should be less than about 10 wt % or less than about 2 wt %.

After performing the spray drying process, a secondary drying step of the powder can be used to remove excess residual solvent, because the presence of solvents can plasticize the solid dispersion by increasing molecular mobility and can result in the development of crystal growth. Following its formation, the solid dispersion can be dried to remove residual solvent using suitable drying processes, such as tray drying, vacuum drying, fluid bed drying, microwave drying, belt drying, rotary drying, and other drying processes known in the art. Preferred secondary drying methods include vacuum drying or tray drying. To minimize chemical degradation during drying, drying can take place under an inert gas such as nitrogen, or can take place under vacuum.

In an embodiment, the formulation obtained is a granulate or a particulate and is combined with one or more extra-granular pharmaceutically acceptable excipients selected from a filler, a diluent, a binder, a surfactant, a disintegrant, a lubricant, a glidant, and a crystallization inhibitor. Conveniently, the extra-granular excipients are selected from a filler, a diluent, a disintegrant, a lubricant and a glidant.

In a convenient embodiment, the method further comprises compressing the composition into a tablet. In an embodiment, the compression force used to form the tablet is less than or equal to 100 MPa, such as less than or equal to 75 MPa.

Optionally, the tablet may be coated by methods well known in the art. The coating may be a pH-independent coating (such as seal coatings or film coatings) or a pH-dependent coating (such as enteric coatings or modified release coatings).

In an embodiment, there is provided a pharmaceutical composition obtained by, or obtainable by, a method according to the second aspect of the invention.

It is to be understood that the rate of release of Compound 1 may vary, for example a short “initial burst” of active agent may be observed shortly after administration followed by a period of lower release. However, prolonged release means that plasma levels of Compound 1 can be kept at therapeutically effective plasma concentrations throughout most if not all of the duration of a prolonged release period.

In some embodiments, compositions of the present invention are able to provide low initial burst and steady and continuous high plasma levels of Compound 1 for a prolonged period after a single oral administration, i.e. without the need for a loading dose.

Advantageously, the applicants have found that the prolonged release provided by certain compositions of the invention, and the resultant continuous plasma levels at therapeutically effective concentration over a prolonged period allow the possibility of using lower than expected doses of Compound 1.

Advantageously, compositions of the invention are able to maintain a therapeutically effective plasma concentration of the drug throughout a prolonged period.

Helicase-primase inhibitors are typically subject to plasma protein binding. Dependent on the extent of the plasma protein binding, the free fraction (unbound active agent) may be low relative to the protein bound fraction. For example, pritelivir is typically subject to 97-98% protein binding, so the free fraction may only be 2-3% of the total plasma concentration. In an embodiment, plasma concentrations referred to herein refer to the unbound plasma concentrations.

In an embodiment, the pharmaceutical composition according to the present invention, after oral administration to a subject in need of treatment thereof, achieves and then maintains in the subject a geometric mean total (unbound and protein bound) plasma concentration of Compound 1 of at least 1000 ng/ml, such as at least 1100 ng/ml, throughout most if not all of at least a 5 day period, conveniently a 7 day period, yet more conveniently a 14 day period or a 28 day period. The oral administration may initially involve regular administration, e.g. by way of daily dosing for a period such as 1 to 7 days, to achieve steady-state plasma concentrations. Conveniently, the pharmaceutical composition according to the present invention, after oral administration, and once steady-state levels have been in achieved in a subject in need of treatment thereof, maintains in the subject a geometric mean total (unbound and protein bound) plasma concentration of Compound 1 of at least 1100 ng/ml, throughout most if not all of at least a 5 day period, such as a 7 day period, conveniently a 14 day period and yet more conveniently a 28 day period.

In some convenient embodiments, compositions of the present invention are able to provide steady and continuous high plasma levels of Compound 1 for a prolonged period after just a single oral administration, i.e. without the need for a loading dose. Conveniently, such pharmaceutical composition according to the present invention, after oral administration, maintain in a subject in need of treatment, a geometric mean total (unbound and protein bound) plasma concentration of Compound 1 of at least 1100 ng/ml, throughout most if not all of at least a 5 day period, such as a 7 day period, conveniently a 14 day period and yet more conveniently a 28 day period.

Conveniently, the pharmaceutical composition according to the present invention is administered on a once weekly basis. Conveniently, the pharmaceutical composition according to the present invention is administered on a once every two week basis. Conveniently, the pharmaceutical composition according to the present invention is administered on a once monthly basis.

It has been surprisingly found that certain compositions of the present invention are able to provide higher plasma levels of Compound 1 for a prolonged period when administered in connection with the intake of food. This allows the potential for possibility using lower than expected doses of Compound 1. As such, in some embodiments, compositions of the present invention are administered in connection with the intake of food. Conveniently, these compositions are oral suspensions or oral tablets comprising the solid dispersion. Conveniently, these compositions are oral suspensions comprising the solid dispersion. Conveniently, these compositions are oral tablets comprising the solid dispersion.

### Therapeutic Uses and Applications

The present invention provides a method for treating a herpes virus (conveniently a HSV) infection in a subject in need thereof, the method comprising administering a therapeutically effective amount of Compound 1, or a pharmaceutically acceptable salt thereof, to the subject wherein Compound 1 or a pharmaceutically acceptable salt thereof, is formulated as a pharmaceutical composition according to the first aspect of the invention.

The present invention also provides a pharmaceutical composition according to the first aspect of the invention comprising Compound 1 or a pharmaceutically acceptable salt thereof, for use as a medicament.

The present invention also provides a pharmaceutical composition according to the first aspect of the invention comprising Compound 1 or a pharmaceutically acceptable salt thereof for use in the treatment of a herpes virus (conveniently a HSV) infection in a subject in need thereof.

The present invention also provides the use of a pharmaceutical composition according to the first aspect of the invention comprising Compound 1 or a pharmaceutically acceptable salt thereof for the manufacture of a medicament for the treatment of a herpes virus (conveniently a HSV) infection in a subject in need thereof.

In a particular embodiment, the present invention provides a method for inhibiting HSV replication in a subject in need thereof, the method comprising administering a therapeutically effective amount of Compound 1, or a pharmaceutically acceptable salt thereof, to the subject wherein Compound 1 or a pharmaceutically acceptable salt thereof, is formulated as a pharmaceutical composition according to the first aspect of the invention.

In one embodiment, the present invention also provides a method for reducing the likelihood or severity of symptoms of a HSV infection in a subject in need thereof, the method comprising administering a therapeutically effective amount of Compound 1, or a pharmaceutically acceptable salt thereof, to the subject wherein Compound 1 or a pharmaceutically acceptable salt thereof, is formulated as a pharmaceutical composition according to the first aspect of the invention.

In one embodiment, the present invention provides a method for inhibiting the development or progression of a disease or disorder caused by, or associated with, HSV infection, in a subject in need thereof, the method comprising administering a therapeutically effective amount of Compound 1, or a pharmaceutically acceptable salt thereof, to the subject wherein Compound 1 or a pharmaceutically acceptable salt thereof, is formulated as a pharmaceutical composition according to the first aspect of the invention.

In one embodiment, the present invention provides a method for suppressing recurrence of HSV symptoms or outbreaks in a subject in need thereof, the method comprising administering a therapeutically effective amount of Compound 1, or a pharmaceutically acceptable salt thereof, to the subject wherein Compound 1 or a pharmaceutically acceptable salt thereof, is formulated as a pharmaceutical composition according to the first aspect of the invention.

In an embodiment, the present invention provides a pharmaceutical composition according to the first aspect of the invention comprising Compound 1 or a pharmaceutically acceptable salt thereof for use in suppressing the recurrence of HSV symptoms or outbreaks in a subject in need thereof.

In an embodiment, the present invention provides the use of a pharmaceutical composition according to the first aspect of the invention comprising Compound 1 or a pharmaceutically acceptable salt thereof for the manufacture of a medicament for the suppression of the recurrence of HSV symptoms or outbreaks in a subject in need thereof.

In one embodiment, the present invention provides a method for treating or preventing a disease or disorder caused by, or associated with, HSV infection, in a subject in need thereof, the method comprising administering a therapeutically effective amount of Compound 1, or a pharmaceutically acceptable salt thereof, to the subject wherein the Compound 1, or a pharmaceutically acceptable salt thereof, is formulated as a pharmaceutical composition according to the first aspect of the invention. In a particular embodiment, the disease or disorder caused by, or associated with, HSV infection, is selected from herpes labialis (e.g., oro-labial cold sores or Whitlow's), Herpes genitalis, HSV-related keratitis, HSV-related encephalitis, pneumonia, herpes gladiatorum, primary HSV gingivostomatitis, Mollaret's meningitis, and Bell's palsy.

In a particular embodiment, the disease or disorder caused by, or associated with, HSV infection, is selected from herpes labialis (oro-labial cold sores or Whitlow's) or genital herpes. In one embodiment, the disease or disorder is recurrent herpes labialis or recurrent genital herpes. Individuals with a history of multiple recurrences of herpes labialis or recurrent genital herpes, e.g. HSV which recurs six times or more annually, may be regarded as having recurrent HSV.

In one embodiment, the herpes virus being treated is HSV2. In a further embodiment, the herpes virus being treated is HSV2 and the subject in need of the treatment has HSV2 recurrent genital herpes.

In one embodiment, the herpes virus being treated is HSV1. In yet a further embodiment, both herpes virus HSV1 and HSV2 are being treated.

In one embodiment, the herpes virus being treated is resistant to nucleosidic anti-viral therapy. In one embodiment, the nucleosidic antiviral therapy is selected from the group consisting of acyclovir, penciclovir, famciclovir, ganciclovir and valacyclovir.

In one embodiment, the herpes virus infection being treated is resistant to nucleosidic antiviral therapy, e.g., acyclovir-resistant mucocutaneous HSV infection. In a further embodiment, the HSV infection being treated is a mucocutaneous HSV infection resistant to therapy with antiviral therapy with nucleoside analogues, such as acyclovir, penciclovir, famciclovir, ganciclovir or valacyclovir.

In a particular embodiment, the subject in need of the methods disclosed herein, is immunocompromised. The subject may be immunocompromised due to conditions including HIV infection, cancer, hematopoietic cell or solid organ transplantation, chronic glucocorticoid use or a genetic immunodeficiency.

In a particular embodiment, the subject in need of the methods disclosed herein, is a neonate or an infant.

In a particular embodiment, the subject is a herpes-positive patient.

In a particular embodiment, the subject in need of the methods disclosed herein, has acyclovir-resistant mucocutaneous HSV infection. This subject may have been diagnosed with this condition on the basis of clinical failure, e.g., no improvement after oral or iv doses for at least 7 days with approved doses of acyclovir.

In a particular embodiment, the subject in need of the methods disclosed herein, has a primary genital HSV-related herpes infection. In one embodiment, the subject in need of the methods disclosed herein, has severe or progressive genital HSV-related herpes infection.

In a particular embodiment, the pharmaceutical compositions according to the first aspect of the invention can reduce recurrence of HSV infections (i.e., provide a suppressive therapy) causing diseases or disorders, such as herpes labialis or genital herpes. In one embodiment, the reduction in the number of recurrences of lesions over a period of one year can be reduced by 20, 30, 40, 50, 75, 90 or 95% or more. Conveniently, the rate of lesions over one year can be reduced by 90% or more.

In a particular embodiment, the pharmaceutical compositions according to the first aspect of the invention can reduce the duration of recurrent episodes of HSV infection, e.g., by one or more days, e.g., at least 2, 3, 4, 5, 14, 21 or 28 days.

In a particular embodiment, the pharmaceutical compositions according to the first aspect of the invention can reduce time to healing of lesions (e.g., time to full recovery of lesions) and duration of symptoms resulting from HSV infections in diseases or disorders, such as herpes labialis or genital herpes. The time to lesion healing may be defined as complete epithelization of mucocutaneous HSV lesion(s) within the treatment period and no appearance of new lesions, e.g., as assessed by a physician.

In one embodiment, the pharmaceutical compositions according to the first aspect of the invention can reduce pain or pain intensity (for example, at a lesion site) caused as a consequence of HSV infections in diseases or disorders, such as herpes labialis or genital herpes.

In a particular embodiment, the pharmaceutical compositions according to the first aspect of the invention can reduce viral shedding or reduce the rate of viral shedding in individuals with frequently recurring HSV, e.g., genital HSV2. For example, a within-subject genital HSV mucocutaneous shedding rate can be measured by taking swabs of skin and mucosa and for HSV detection, e.g. by analysing samples for HSV DNA with a real-time, quantitative, fluorescent polymerase-chain-reaction (PCR) assay. The frequency of HSV2 detection (the viral shedding rate) can be defined as the number of days with a genital swab that was positive for HSV divided by the total number of days on which genital swabs were obtained. Reduction in the HSV shedding rate among subjects receiving the compositions of the invention relative to the shedding rate among subjects receiving placebo or other treatments can be compared. The quantity of HSV in positive swabs and the frequency of genital lesions and shedding episodes can also be monitored.

In a particular embodiment, the present invention provides a method for reducing (or substantially supressing or eliminating) break-through HSV shedding in a subject in need thereof, the method comprising administering a therapeutically effective amount of Compound 1, or a pharmaceutically acceptable salt thereof, to the subject wherein the Compound 1 or a pharmaceutically acceptable salt thereof, is formulated as a pharmaceutical composition according to the first aspect of the invention.

In an embodiment, the present invention provides a pharmaceutical composition according to the first aspect of the invention comprising Compound 1 or a pharmaceutically acceptable salt thereof for use in reducing (or substantially supressing or eliminating) break-through HSV shedding in a subject in need thereof.

In an embodiment, the present invention provides the use of a pharmaceutical composition according to the first aspect of the invention comprising Compound 1 or a pharmaceutically acceptable salt thereof for the manufacture of a medicament for the reduction (or substantial suppression or elimination) of break-through HSV shedding in a subject in need thereof.

In a particular embodiment, the present invention provides a method for preventing transmission of an infectious disease caused by HSV, the method comprising administering a therapeutically effective amount of Compound 1, or a pharmaceutically acceptable salt thereof, to the subject wherein the Compound 1 or a pharmaceutically acceptable salt thereof, is formulated as a pharmaceutical composition according to the first aspect of the invention.

In a particular embodiment, the present invention provides a method for reducing side effects observed when Compound 1, or a pharmaceutically acceptable salt thereof, is administered via an oral route of administration to a subject with an infectious disease caused by HSV, the method comprising administering a therapeutically effective amount of Compound 1, or a pharmaceutically acceptable salt thereof, to the subject wherein the Compound 1 or a pharmaceutically acceptable salt thereof, is formulated as a pharmaceutical composition according to the first aspect of the invention.

For use in accordance with the present invention, the appropriate dosage is expected to vary depending on, for example, the nature and severity of the infection to be treated and is within the purview of the treating physician. Usually, an indicated administration dose may be in the range between about 0.1 to about 1000 μg/kg body weight. In some cases, the administration dose of the compound may be less than 400 μg/kg body weight. In other cases, the administration dose may be less than 200 μg/kg body weight. In yet other cases, the administration dose may be in the range between about 0.1 to about 100 μg/kg body weight. In an embodiment, the therapeutically effective amount of the Compound 1 or a pharmaceutically acceptable salt thereof, is about 5 mg to about 900 mg, such as about 40 mg to 600 mg, conveniently such as 100 mg to about 600 mg. In an embodiment, the therapeutically effective amount of the Compound 1 or a pharmaceutically acceptable salt thereof, is about 40 mg to about 100 mg, such as about 50 mg.

Advantageously, the applicants have found that the prolonged release provided by the compositions of the invention and the resultant steady plasma levels kept at a therapeutically effective plasma concentration over a prolonged period after administration allow the possibility of using lower than expected doses.

The dose, for example after steady-state levels have been achieved, may be conveniently administered only once every 5 days or less, once every week or less, every two weeks or less, once a month or less, once every two or three months or less. Conveniently, the dose may be conveniently administered once a week, once every two weeks, once a month or once every two months.

In a convenient embodiment, the unit dosage form of the pharmaceutical composition is a tablet. In a convenient embodiment, the unit dosage form of the pharmaceutical composition comprises about 1 mg to about 300 mg of Compound 1, or a pharmaceutically acceptable salt thereof. In a convenient embodiment, the unit dosage form of the pharmaceutical composition comprises about 25 mg to about 100 mg of Compound 1, or a pharmaceutically acceptable salt thereof, such as about 50 mg.

Advantageously, applicants have found that the steady plasma levels kept at a therapeutically effective plasma concentration over a prolonged period after administration may be enhanced by administration of certain compositions of the invention in connection with the intake of food.

Oral administration of Compound 1, or a pharmaceutically acceptable salt thereof, in connection with the intake of food allows improvement of the pharmacokinetics of the helicase-primase inhibitor, relative to the oral pharmacokinetics when the compound is administered in the fasted state. This treatment approach offers a number of potential benefits. For example, the treatment approach offers the possibility to administer lower doses of the helicase-primase inhibitor, thereby reducing the incidence and/or extent of drug related adverse effects, lowering the costs of the drug treatment therapy, and reducing pill burden and/or dosing frequency, which in turn aids patient use and compliance.

In a convenient embodiment, the composition of the present invention is administered in connection with the intake of food.

In one embodiment, the present invention provides a method for treating a herpes virus (conveniently a HSV) infection in a subject in need thereof, the method comprising orally administering to the subject, in connection with the intake of food, a therapeutically effective amount of Compound 1, or a pharmaceutically acceptable salt thereof, to the subject wherein Compound 1 or a pharmaceutically acceptable salt thereof, is formulated as a pharmaceutical composition according to the first aspect of the invention.

The term “food” shall be understood to cover any edible foodstuff having a nutritional value as an energy supplier. Thus the food can be a solid, semi-solid or liquid substance comprising one or more of the basic ingredients, i.e. carbohydrates, fats and proteins. The Compound 1, or a pharmaceutically acceptable salt thereof, is considered to be administered in “connection with the intake of food” if the Compound 1, or a pharmaceutically acceptable salt thereof, is administered at a time point shortly before the start of food intake, during the food intake or in a relatively short time after the food intake. In one embodiment, Compound 1, or a pharmaceutically acceptable salt thereof, is administered at a time point which is in the range defined by 30 minutes before starting the food intake and 2 hours after starting the food intake. Conveniently, Compound 1, or a pharmaceutically acceptable salt thereof, is administered during the food intake or within 1 hour after starting the food intake. More conveniently, Compound 1, or a pharmaceutically acceptable salt thereof, is administered within 30 minutes after starting food intake.

In one embodiment, Compound 1, or a pharmaceutically acceptable salt thereof, is administered at substantially the same time as the food intake. Conveniently, Compound 1, or a pharmaceutically acceptable salt thereof, is administered with food or within 30 minutes of food.

The food can be any suitable food, for example a meal. In one embodiment, a “meal” refers to one of the three substantial intakes of food typical for an individual during a typical day. In one aspect, a “meal” involves the intake of at least 400, 600, 800 or 1000 calories, although the amount of food intake may vary according to the size, weight and general health of the individual. In one embodiment, the term “meal” refers to a meal as defined by the FDA food effect test guidelines and can include a high-fat meal or a low-fat meal. In one embodiment, the food is a high-fat meal. As used herein, the term “high-fat meal” refers generally to a meal wherein at least about 50% of the calories provided are from fat and the meal has a total calorie content of at least about 700 Kcal. In one embodiment, the food is a low-fat meal. In one embodiment, the low-fat meal may be defined as a meal wherein about 25% of the calories provided are from fat and the meal has a total calorie content of 400-600 Kcal.

It is to be understood that the quantity and calorific value of a meal (e.g. a low-fat meal or a high-fat meal) required to achieve a desirable level of improvement in the oral pharmacokinetics of the helicase-primase inhibitor may vary based on the age and/or weight of a subject, e.g. a human patient.

In one embodiment, oral administration of Compound 1, or a pharmaceutically acceptable salt thereof, in connection with the intake of food, provides an increase in any one, two, three or four of the following parameters: Cmax, Tmax and AUC, as compared to administration of Compound 1, or a pharmaceutically acceptable salt thereof, to a subject in the fasted state.

### Combinations

Pharmaceutical compositions of the present invention may be administered alone as a sole therapy or can be administered in addition with one or more other substances and or treatments. Such conjoint treatment may be achieved by way of simultaneous, sequential or separate administration of the individual components of the treatment.

Also contemplated herein are methods that include administering a second active agent. For example, in addition to being infected with HSV, a subject or patient can further have HSV infection-related co-morbidities, i.e., diseases and other adverse health conditions associated with, exacerbated by, or precipitated by being infected with HSV. Contemplated herein are also disclosed pharmaceutical compositions in combination with at least one other agent that has previously been shown to treat these HSV-infection-related conditions. Such conjoint treatment may be achieved independently (by way of simultaneous, sequential or separate administration of the individual components of the treatment) and/or via pharmaceutical compositions of the present invention that include a second active agent.

Therefore, provided herein is a method for treating or preventing HSV infection in a subject in need thereof, the method comprising administering a therapeutically effective amount of Compound 1, or a pharmaceutically acceptable salt thereof, to the subject, wherein the Compound 1 or a pharmaceutically acceptable salt thereof is formulated as a pharmaceutical composition according to the first aspect of the invention, and co-administering to the subject a therapeutically effective amount of an additional therapeutic agent.

In an embodiment, the additional therapeutic agent is selected from one or more of the following agents:
i. nucleoside polymerase inhibitors, such as acyclovir, valacyclovir, famciclovir, penciclovir and ganciclovir;
ii. pyrophosphate polymerase inhibitors, such as foscarnet;
iii. saturated aliphatic alcohols, such as docosanol;
iv. agents such as idoxuridine, trifluridine and vidarabine;
V. a corticosteroid; and
vi. other helicase-primase inhibitors, such as amenamevir.

In some cases, a disclosed pharmaceutical composition according to the first aspect of the invention may be administered as part of a combination therapy in conjunction with one or more antivirals, including nucleoside analogues such as acyclovir, foscarnet, ganciclovir or penciclovir or the respective prodrugs valacyclovir or famciclovir.

In some embodiments, the first and second amounts together comprise a pharmaceutically effective amount. The first amount, the second amount, or both may be the same, more, or less than effective amounts of each compound administered as monotherapies. Therapeutically effective amounts of a disclosed compound and antiviral may be co-administered to the subject, i.e., administered to the subject simultaneously or separately, in any given order and by the same or different routes of administration. In some instances, it may be advantageous to initiate administration of Compound 1 first, for example one or more days or weeks prior to initiation of administration of the antiviral. Moreover, additional drugs may be given in conjunction with the above combination therapy.

### Kits

In one embodiment, the pharmaceutical compositions and methods described herein provide kits for the treatment of disorders, such as the one described herein. These kits comprise a pharmaceutical composition described herein in a container and, optionally, instructions teaching the use of the kit according to the various methods and approaches described herein. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

In one embodiment, the present invention provides a kit comprising a pharmaceutical composition of the invention for oral administration to a subject and instructions, e.g. printed instructions, to administer the composition comprising Compound 1, or a pharmaceutically acceptable salt thereof, in connection with the intake of food (conveniently with or shortly after food). In one embodiment of this aspect, the instructions also inform the subject that oral administration of Compound 1 or a pharmaceutically acceptable salt thereof, with food results in an improvement in oral pharmacokinetics and/or systemic exposure and/or oral bioavailability of Compound 1, as compared to administration without food.

The pharmaceutical compositions of the invention may be utilized for diagnostics and as research tools.

Besides being useful for human treatment, pharmaceutical compositions of the invention, may be useful for veterinary treatment of companion animals, exotic animals and farm animals, including mammals, rodents, and the like. Conveniently, such animals include horses, dogs and cats.

The invention is illustrated below by the following non-limiting examples.

### EXAMPLES

### Materials and Methods

TABLE 1
Materials and Equipment

| | Trade Name or | Abbreviation or | Manu- |
|---|---|---|---|
| Material and Equipment | Model | Equipment ID | facturer |
| Acetone | N/A | Acetone | EMD |
| Water | N/A | H2O | MQ-1 |
| Methanol | N/A | MeOH | EMD |
| Tetrahydrofuran | N/A | THF | Fisher |
| Methylene Chloride | N/A | DCM | Fisher |
| N-methyl pyrrolidone | N/A | NMP | Fisher |
| Ethanol | N/A | EtOH | Fisher |
| Ethyl acetate | N/A | EtOAc | Fisher |
| Hypromellose acetate | AQOAT ®-MG | HPMCAS-MG | Shin- |
| succinate MG grade | | | Etsu |
| Hypromellose acetate | AQOAT ®-LG | HPMCAS-LG | Shin- |
| succinate LG grade | | | Etsu |
| Hypromellose acetate | AQOAT ®-HG | HPMCAS-HG | Shin- |
| succinateHG grade | | | Etsu |
| Poly(butyl methacrylate- | Eudragit ® E | N/A | Evonik |
| co-(2-demethyl - | PO | | |
| aminoethyl) | | | |
| methacrylate-co-methyl | | | |
| methacrylate) 1:2:1 | | | |
| Hypromellose E5LV | Hypromellose | HPMC E5LV | DOW |
| PEG 6000/vinyl | Soluplus ® | N/A | BASF |
| caprolactam/vinyl | | | |
| acetate copolymer | | | |
| Polyvinylpyrrolidone/ | Kollidon ® VA | PVP-VA64 | BASF |
| Vinyl acetate copolymer | 64 | | |
| Polyethylene glycol 300 | N/A | PEG 300 | Sigma |
| D-α-Tocopherol | N/A | TPGS | Sigma |
| polyethylene | | | |
| glycol succinate | | | |
| Poloxamer 188 | Kolliphor ® P | N/A | BASF |
| | 188 | | |
| Polyvinylpyrrolidone | Povidone K30 | PVP-K30 | Sigma |
| K30 grade | | | |
| Spray Dryer | Yamato DL410 | DL410 | Yamato |
| Spray Dryer | B-290 | Buchi | Buchi |
| Spray Dryer | PSD-3 | PSD-3 | Gea |

Solid dispersions were characterized using one or more of the following analytical methods: differential scanning calorimetry (DSC), X-ray powder diffraction (XRPD), residual solvents by gas chromatography headspace sampling (GC-HS), polarized light microscopy (PLM), assay and impurities by high-performance liquid chromatography (HPLC) and water content by Karl Fisher titration (KF).

I. Differential Scanning calorimetry (DSC)

DSC was performed using a TA Instruments Discovery DSC2500 differential scanning calorimeter equipped with a TA instruments Refrigerated Cooling System 90 operating in either modulated or ramp mode. DSC was used to measure thermodynamic events and characteristics of compounds and subsequent solid dispersions. Events that need to be observed include the glass transition temperature (T_{g}), defined as the temperature at which amorphous materials transition from a low mobility glassy state to a high mobility rubbery state, cold crystallization (T_{c}), defined as a crystallization event at a temperature lower than the melt temperature, and melting temperature (Tₘ). Samples were placed in non-hermetic aluminium pans and heated with a heating rate of 10° C./min from room temperature ramping up to 300° C. The system was purged by nitrogen flow at 50 mL/min to ensure inert atmosphere through the course of measurement. Samples were also analysed by MDSC. A summary of DSC and MDSC analysis parameters can be found Table 2 and 3.

TABLE 2
DSC Analysis Parameters

| Parameter | Value |
|---|---|
| Instrument | TA Discovery DSC2500 |
| Sample Pans | Tzero Al, Non-hermetic |
| Temp. Range | RT-300° C. |
| Heating Rate | 10° C./min |
| Scanning Mode | Ramp |
| Purge gas | Nitrogen |

TABLE 3
MDSC Analysis Parameters

| Parameter | Value |
|---|---|
| Instrument | TA Discovery DSC2500, |
| Sample Pans | Tzero Al, Non-hermetic |
| Temp. Range | −50-300° C. |
| Heating Rate | 2° C./min |
| Scanning Mode | Modulated |
| Modulation Frequency | 60 s |
| Modulation Amplitude | 1° C. |

### II. Thermal Gravimetric Analysis (TGA)

The sample weight change during heating of compound was monitored by TGA. The sample was exposed to a temperature gradient starting at room temperature and increased by 10° C./min up to a final temperature of 300° C. A precision balance recorded the weight change of the sample as a function of temperature. TGA analysis parameters are displayed in Table 4.

TABLE 4
TGA Analysis Parameters

| Parameters | Value |
|---|---|
| Instrument: | TA Discovery TGA500 |
| Temp. Range: | RT-300° C. |
| Method: | Ramp |
| Heating Rate: | 10.0° C./min |
| Pan | Aluminium, Open |
| Purge gas | Nitrogen |

### III. X-Ray Powder Diffraction (XRPD)

XRPD was performed using a Rigaku Smartlab SE X-ray diffractometer to evaluate the crystallinity of bulk API and subsequent solid dispersion materials. Amorphous materials give an “amorphous halo” diffraction pattern, absent of discrete peaks that would be found in a crystalline material. Samples were irradiated with monochromatized Cu Kα radiation and analyzed between 3° and 40° with a continuous scanning mode. Samples were rotated during analysis to minimize preferred orientation effects. A summary of XRPD analysis parameters can be found in Table 5.

TABLE 5
XRPD Analysis Parameters

| Parameter | Value |
|---|---|
| Instrument | Rigaku Smartlab SE |
| X-Ray Wavelength | Cu, Kα, Kα1(Å):1.540598, Kα2(Å): 1.544426 |
| | Kα2: Kα1 intensity ratio: 0.50 |
| X-Ray Tube Setting | 40 kV, 15 mA, Scan Mode 1D |
| Scan Range (20) | 3-40° |
| Step Size (20) | 0.02° |
| Scan Speed (20) | 10°/min |
| Scan Type | Scan Mode 1D D/tex Ultra 250 |
| Rotation | // |
| Holder | ASC-48 auto sample changer attachment |
| Receiving Slit | 20 mm (Soller slit: 2.5 degree; length limiting |
| Width | slit: 10 mm) |
| Divergent Slit | 1/2 degree (incident slit; incident soller 2.5 |
| Width | degree) |
| Knife Edge Width | // |

### IV. Amorphous Content by Polarized Light Microscopy (PLM)

Compounds, dry solid dispersions and solid dispersion suspensions were analyzed by PLM. Samples were dispersed with methyl silicone oil on a glass slide and observed by PLM. Samples were inspected using a Olympus BX53 microscope.

### V. Water Content by Coulometric Karl Fisher (KF) Titration

Samples were analyzed for water content by a Metrohm 915 Ti-Touch Karl Fischer Coulometric Titrator. About 100 mg samples were sealed in 6 mL crimp vials followed by measurement of water content with the following parameters: Reagent Hydranal Coulomat AG-Oven, oven temperature 130° C. and sample extraction time 300 seconds.

### VI. Assay and Impurities Analysis by HPLC

Assay and impurities of solid dispersion samples were evaluated using an experimental HPLC method (Table 6).

TABLE 6
HPLC Parameters for Assay and Impurities

| Parameter | Value | | |
|---|---|---|---|
| Column | Waters X-Bridge C18 (4.6 × 150 mm | | |
| | 5 μm) PN 186003116 | | |
| Mobile Phase A | 10 mM Ammonium acetate buffer solution | | |
| Mobile Phase B | Acetonitrile | | |
| Diluent | DMSO: ACN = 5: 95 (v/V) | | |
| Program Type | Gradient | | |
| | | % Mobile | % Mobile |
| Gradient Program | Time (min) | Phase A | Phase B |
| | 0.0 | 70 | 30 |
| | 4.0 | 55 | 45 |
| | 10.0 | 45 | 55 |
| | 12.0 | 40 | 60 |
| | 18.0 | 20 | 80 |
| | 20.0 | 20 | 80 |
| | 20.1 | 70 | 30 |
| | 25.0 | 70 | 30 |

| | |
|---|---|
| Flow Rate | 1.0 mL/min |
| Column Temperature | 40° C. |
| Sample Temperature | Room Temperature |
| Injection Volume | 5 μL |
| Detection Method | UV |
| Detection Wavelength | 286 nm |

### VII. Residual Solvent by Gas Chromatography Headspace Sampling

The residual solvent content of solid dispersions was measured by GC-HS after secondary drying. Measurements were made using an Agilent 8890B with FID detector series GC equipped with an Agilent 7697A headspace sampler. A 30 m×0.32 mm×1.8 u capillary column with 6% cyanopropylphenyl 94% dimethylpolysiloxane GC column was used for the testing. GC samples were prepared by dissolving ~100 mg sample in 5 mL N-methylpyrrolidone (NMP). The GC method parameters are summarized in Table 7.

TABLE 7
GC-HS Analysis Parameters

| Parameter | Value | | | |
|---|---|---|---|---|
| Injector Temperature | 230° C. | | | |
| Split Ratio | 10:1 | | | |
| Carrier Gas | Nitrogen | | | |
| Detector | FID | | | |
| Detector Temperature | 250°C | | | |
| Makeup (N2) Flow | 25 mL/min | | | |
| H2 Flow | 30 mL/min | | | |
| Air Flow | 300 mL/min | | | |
| Run Time | 17.7 min | | | |
| Column Tem- | | Rate | Value | Hold time |
| perature Program | | (° C./min) | ((° C./) | (min) |
| | Initial | / | 45 | 0.2 |
| | Ramp 1 | 2 | 50 | 0.0 |
| | Ramp 2 | 10 | 100 | 0.0 |
| | Ramp 3 | 30 | 250 | 5.0 |

### LC-MS Analysis of Plasma Samples

The LC-MS methodology and conditions used to analyse the helicase-primase inhibitor, Compound 1, in plasma samples from the oral PK studies (Examples 5-9) are described in Tables 8 to 11.

TABLE 8
LC-MS Parameters for Compound 1 quantification in rat
plasma samples from Single Oral Dose PK study

| Parameter | Value | | |
|---|---|---|---|
| Instrument | LC-MS/MS-47 (Triple Quad 6500+) | | |
| MS conditions | Positive, ESI | | |
| | Internal standard: diclofenac | | |
| | Compound ID | Parent (m/z)/ | |
| | | Daughter (m/z) | |
| | Compound 1 | Q1/Q3 Masses: | |
| | | 465.10/232.20 Da | |
| | Diclofenac | Q1/Q3 Masses: | |
| | | 296.20/214.20 Da | |
| Column | Xbridge BEH C18 | | |
| | (2.1 × 50 mm, 2.5 μm) | | |
| Flow rate | 0.60 mL/min | | |
| Column Temperature | 50° C. | | |
| Sample diluent | MeOH:H₂O 50:50 (v:v) | | |
| Mobile Phase A | Water/0.025% Formic acid/ | | |
| | 1 mM Ammonium Acetate | | |
| Mobile Phase B | Methanol/5mM Ammonium | | |
| | Acetate | | |
| Program Type | Gradient | | |
| | | % Mobile | % Mobile |
| Gradient Program | Time (min) | Phase A | Phase B |
| | 0.20 | 90 | 10 |
| | 0.40 | 40 | 60 |
| | 1.40 | 10 | 90 |
| | 1.80 | 10 | 90 |
| | 1.81 | 90 | 10 |
| | 2.20 | Stop | Stop |

| | |
|---|---|
| Sample Temperature | Room Temperature |
| Injection Volume | 1.0 μL |
| MS Detection Mode | Multiple reaction monitoring |
| | (MRM) |
| Retention time | 1.49 min (Compound 1) |
| | 1.56 min (Diclofenac) |

TABLE 9
LC-MS Parameters for Compound 1 quantification in dog
plasma samples from Single Oral Dose PK study

| Parameter | Value | | |
|---|---|---|---|
| Instrument | LC-MS/MS-47 (Triple Quad 6500+) | | |
| MS conditions | Positive, ESI | | |
| | Internal standard: diclofenac | | |
| | Compound ID | Parent (m/z)/ | |
| | | Daughter (m/z) | |
| | Compound 1 | Q1/Q3 Masses: | |
| | | 465.10/232.20 Da | |
| | Diclofenac | Q1/Q3 Masses: | |
| | | 296.20/214.20 Da | |
| Column | Xbridge BEH C18 | | |
| | (2.1 × 50 mm, 2.5 μm) | | |
| Flow rate | 0.60 mL/min | | |
| Column Temperature | 50° C. | | |
| Sample diluent | MeOH: H₂O 50:50 (v:v) | | |
| Mobile Phase A | Water/0.025% Formic acid/ | | |
| | 1 mM Ammonium Acetate | | |
| Mobile Phase B | Methanol/5mM Ammonium | | |
| | Acetate | | |
| Program Type | Gradient | | |
| | | % Mobile | % Mobile |
| Gradient Program | Time (min) | Phase A | Phase B |
| | 0.20 | 90 | 10 |
| | 0.40 | 40 | 60 |
| | 1.40 | 10 | 90 |
| | 1.80 | 10 | 90 |
| | 1.81 | 90 | 10 |
| | 2.20 | Stop | Stop |

| | |
|---|---|
| Sample Temperature | Room Temperature |
| Injection Volume | 1.0 μL |
| MS Detection Mode | Multiple reaction monitoring |
| | (MRM) |
| Retention time | 1.44 min (Compound 1) |
| | 1.49 min (Diclofenac) |

TABLE 10
LC-MS Parameters for Compound 1 quantification in rat or
dog plasma samples from Seven Day Oral Dose PK studies

| Parameter | Value | | |
|---|---|---|---|
| Instrument | LC-MS/MS-47 (Triple Quad 6500+) | | |
| MS conditions | Positive, ESI | | |
| | Internal standard: diclofenac | | |
| | Compound ID | Parent (m/z)/ | |
| | | Daughter (m/z) | |
| | Compound 1 | Q1/Q3 Masses: | |
| | | 465.10/232.20 Da | |
| | Diclofenac | Q1/Q3 Masses: | |
| | | 296.20/214.20 Da | |
| Column | Xbridge BEH C18 | | |
| | (2.1 × 50 mm, 2.5 μm) | | |
| Flow rate | 0.60 mL/min | | |
| Column Temperature | 60° C. | | |
| Sample diluent | MeOH:H₂O 50:50 (v:v) | | |
| Mobile Phase A | Water/0.025% Formic acid/ | | |
| | 1 mM Ammonium Acetate | | |
| Mobile Phase B | Methanol/5 mM Ammonium | | |
| | Acetate | | |
| Program Type | Gradient | | |
| | | % Mobile | % Mobile |
| Gradient Program | Time (min) | Phase A | Phase B |
| | 0.20 | 90 | 10 |
| | 0.40 | 40 | 60 |
| | 1.40 | 10 | 90 |
| | 1.80 | 10 | 90 |
| | 1.81 | 90 | 10 |
| | 2.20 | Stop | Stop |

| | |
|---|---|
| Sample Temperature | Room Temperature |
| Injection Volume | 1.0 μL |
| MS Detection Mode | Multiple reaction monitoring |
| | (MRM) |
| Retention time | 1.34 min (Compound 1) |
| | 1.40 min (Diclofenac ) |

TABLE 11
LC-MS Parameters for Compound 1 quantification in dog
plasma samples from Fourteen Day Oral Dose PK study

| Parameter | Value | | |
|---|---|---|---|
| Instrument | LC-MS/MS-47 (Triple Quad 6500+) | | |
| MS conditions | Positive, ESI | | |
| | Internal standard: glipizide | | |
| | Compound ID | Parent (m/z)/ | |
| | | Daughter (m/z) | |
| | Compound 1 | Q1/Q3 Masses: | |
| | | 465.10/232.20 Da | |
| | Glipizide | Q1/Q3 Masses: | |
| | | 446.20/321.10 Da | |
| Column | Xbridge BEH C18 | | |
| | (2.1 × 50 mm, 2.5 μm) | | |
| Flow rate | 0.60 mL/min | | |
| Column Temperature | 50° C. | | |
| Sample diluent | MeOH:H₂O 50:50 (v:v) | | |
| Mobile Phase A | Water/0.025% Formic acid/ | | |
| | 1 mM Ammonium Acetate | | |
| Mobile Phase B | Methanol/5 mM Ammonium | | |
| | Acetate | | |
| Program Type | Gradient | | |
| | | % Mobile | % Mobile |
| Gradient Program | Time (min) | Phase A | Phase B |
| | 0.20 | 90 | 10 |
| | 0.60 | 30 | 70 |
| | 1.50 | 10 | 90 |
| | 2.00 | 10 | 90 |
| | 2.01 | 90 | 10 |
| | 2.50 | Stop | Stop |

| | |
|---|---|
| Sample Temperature | Room Temperature |
| Injection Volume | 1.0 μL |
| MS Detection Mode | Multiple reaction monitoring |
| | (MRM) |
| Retention time | 1.32 min (Compound 1) |
| | 1.24 min (Glipizide) |

The LC-MS methodology and conditions used to analyse Compound 1 in plasma samples from the intravenous injection PK studies (Example 3) are described in Tables 12 to 14.

TABLE 12
LC-MS Parameters for Compound 1 quantification
in rat plasma samples following intra-venous
administration in study described in Example 3

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 | | |
| | (2.1 × 50 mm, 1.7 μm) | | |
| Flow rate | 0.60 mL/min | | |
| Column Temperature | 60° C. | | |
| Sample diluent | MeOH:H₂O 50:50 (v:v) | | |
| Mobile Phase A | Water/0.025% Formic acid/ | | |
| | 1 mM Ammonium Acetate | | |
| Mobile Phase B | Methanol/5 mM Ammonium | | |
| | Acetate | | |
| Program Type | Gradient | | |
| | | % Mobile | % Mobile |
| Gradient Program | Time (min) | Phase A | Phase B |
| | 0.0 | 90 | 10 |
| | 0.20 | 90 | 10 |
| | 1.20 | 10 | 90 |
| | 1.80 | 10 | 90 |
| | 1.81 | 90 | 10 |
| | 2.20 | Stop | stop |

| | |
|---|---|
| Sample Temperature | Room Temperature |
| Injection Volume | 2.0 μL |
| MS Detection Mode | Multiple reaction monitoring |
| | (MRM) |
| Retention time | 1.43 min |

TABLE 13
LC-MS Parameters for Compound 1 quantification
in dog plasma samples following intra-venous
administration in study described in Example 3

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 | | |
| | (2.1 × 50 mm, 1.7 μm) | | |
| Flow rate | 0.60 mL/min | | |
| Column Temperature | 60° C. | | |
| Sample diluent | MeOH:H₂O 50:50 (v:v) | | |
| Mobile Phase A | Water/0.025% Formic acid/ | | |
| | 1 mM Ammonium Acetate | | |
| Mobile Phase B | Methanol/5 mM Ammonium | | |
| | Acetate | | |
| Program Type | Gradient | | |
| | | % Mobile | % Mobile |
| Gradient Program | Time (min) | Phase A | Phase B |
| | 0.0 | 90 | 10 |
| | 0.20 | 98 | 2 |
| | 1.20 | 98 | 2 |
| | 1.80 | 10 | 90 |
| | 1.81 | 90 | 10 |
| | 2.20 | Stop | Stop |

| | |
|---|---|
| Sample Temperature | Room Temperature |
| Injection Volume | 1.0 μL |
| MS Detection Mode | Multiple reaction monitoring |
| | (MRM) |
| Retention time | 1.48 min |

TABLE 14
LC-MS Parameters for Compound 1 quantification in
monkey plasma samples following intra-venous
administration in study described in Example 3

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 | | |
| | (2.1 × 50 mm, 1.7 μm) | | |
| Flow rate | 0.60 mL/min | | |
| Column Temperature | 60° C. | | |
| Sample diluent | MeOH:H₂O 50:50 (v:v) | | |
| Mobile Phase A | Water/0.025% Formic acid/ | | |
| | 1 mM Ammonium Acetate | | |
| Mobile Phase B | Methanol/5 mM Ammonium | | |
| | Acetate | | |
| Program Type | Gradient | | |
| | | % Mobile | % Mobile |
| Gradient Program | Time (min) | Phase A | Phase B |
| | 0.0 | 95 | 5 |
| | 0.30 | 95 | 5 |
| | 1.50 | 10 | 90 |
| | 2.00 | 10 | 90 |
| | 2.01 | 95 | 5 |
| | 2.60 | Stop | stop |

| | |
|---|---|
| Sample Temperature | Room Temperature |
| Injection Volume | 1.0 μL |
| MS Detection Mode | Multiple reaction monitoring |
| | (MRM) |
| Retention time | 1.69 min |

### Example 1: Synthesis of 2-(3-(2′,5′-difluoro-[1,1′-biphenyl]-4-yl)-2-oxotetrahydropyrimidin-1(2H)-yl)-4-methylthiazole-5-sulfonamide (Compound 1)

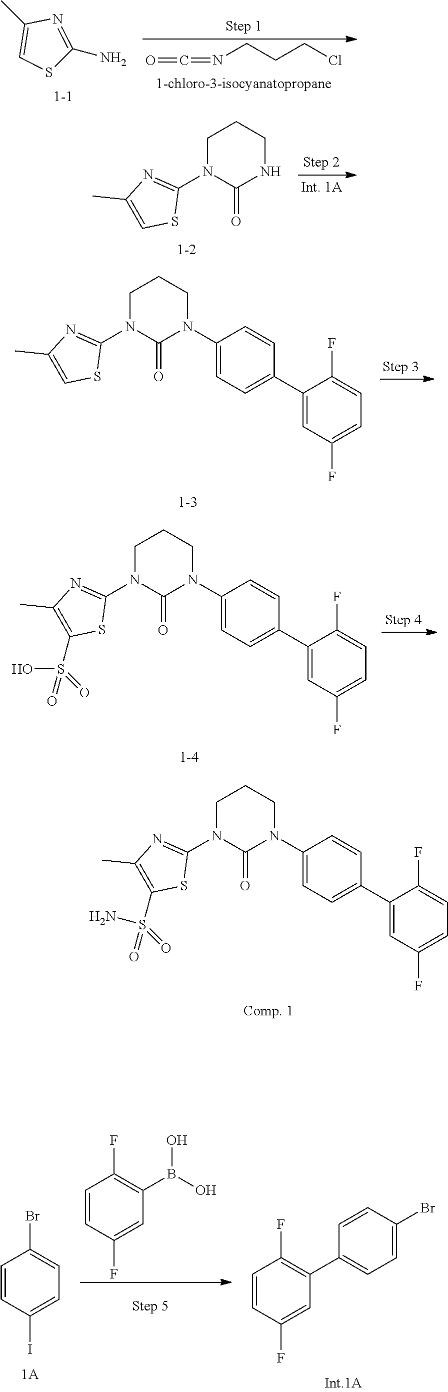

### Synthesis of 1-(4-methylthiazol-2-yl)tetrahydropyrimidin-2(1H)-one (1-2)

A mixture of 2-amino-4-methylthiazole (compound 1-1; 6 g, 52.632 mmol) and 1-chloro-3-isocyanatopropane (6.26 g, 52.632 mmol) in THF (60 mL) was heated at 70° C. for 6 h. To the resulting solution, TBAB (1.7 g, 5.263 mmol) and K₂CO₃ (18.15 g, 131.58 mmol) were added portion wise maintaining the same temperature and stirring continued at 70° C. for 16 h. After completion of the reaction (monitored by TLC), the reaction mixture was diluted with water and extracted with EtOAc. The combined organic layers were dried over anhydrous Na₂SO₄, filtered and concentrated under reduced pressure. The crude compound was purified by CombiFlash® chromatography (eluting with 60-70% EtOAc in heptane) to afford the title compound 1-2 (5.1 g, 49.22%) as an off-white solid. TLC: 70% EtOAc/heptane (R_{f}: 0.5). MS calcd, for Chemical Formula: C₈H₁₁N₃OS: 197.06; Found: 198.17 [M+1]⁺. ¹H NMR (400 MHz, DMSO-d₆) δ 7.30 (s, 1H), 6.60 (s, 1H), 3.99 (t, J=5.4 Hz, 2H), 3.20-3.19 (m, 2H), 2.28 (s, 3H), 1.99-1.89 (m, 2H).

### Synthesis of 1-(2′,5′-difluoro-[1,1′-biphenyl]-4-yl)-3-(4-methylthiazol-2-yl) tetrahydropyrimidin-2(1H)-one (1-3)

To a stirred solution of compound 1-2 (5 g, 25.380 mmol) in 1,4-dioxane (100 mL) were added Int. 1A (8.16 g, 30.456 mmol), K₂CO₃ (8.75 g, 63.45 mmol) followed by CuI (0.96 g, 5.076 mmol) and the resulting reaction mixture was purged under nitrogen for 20 min. To this resulting reaction mixture, 1,2-Dimethylethylenediamine (0.9 g, 10.152 mmol) was added under nitrogen atmosphere. The reaction mixture was heated at 120° C. for 24 h in a sealed tube. After completion of the reaction, the reaction mixture was filtered through Celite® bed and washed with ethyl acetate. The filtrate was diluted with water and, extracted with EtOAc followed by brine. The combined organic layers were dried over anhydrous Na₂SO₄, filtered and concentrated under reduced pressure. The crude compound obtained was purified by CombiFlash® chromatography (eluting with 30-40% EtOAc in heptane) to afford the title compound 1-3 (4.1 g, 41.96%) as an off-white solid. TLC: 50% EtOAc/Heptane (Rx: 0.5). MS calcd, for Chemical Formula: C₂₀H₁₇F₂N₃OS: 385.11; Found: 385.90 [M+1]⁺. ¹H NMR (400 MHz, DMSO-d₆) δ 7.61 (d, J=7.8 Hz, 2H), 7.54-7.35 (m, 4H), 7.35-7.21 (m, 1H), 6.70 (s, 1H), 4.17 (t, J=5.6 Hz, 2H), 3.81 (t, J=4.9 Hz, 2H), 2.26 (s, 3H), 2.24-2.21 (m, 2H).

### Synthesis of 2-(3-(2′,5′-difluoro-[1,1′-biphenyl]-4-yl)-2-oxotetrahydropyrimidin-1(2H)-yl)-4-methylthiazole-5-sulfonic acid (1-4)

To a stirred solution of compound 1-3 (4 g, 10.389 mmol) in dry DCM (40 mL) at 0° C. in an inert atmosphere, chlorosulfuric acid (2.07 mL, 31.168 mmol) was added and the resulting reaction mixture was slowly warmed to room temperature and stirred for 12 h. After completion of the reaction, the reaction mixture was concentrated under reduced pressure to dryness. The crude residue obtained was purified by trituration with diethyl ether. The obtained solid was filtered off and dried in vacuo to afford the title compound 1-4 (3.35 g, crude) as an off-white solid. TLC: 100% EtOAc (R_{f}: 0.2). MS calcd, for Chemical Formula: C₂₀H₁₇F₂N₃O₄S₂: 465.06; Found: 466 [M+1]⁺.

### Synthesis of 2-(3-(2′,5′-difluoro-[1,1′-biphenyl]-4-yl)-2-oxotetrahydropyrimidin-1(2H)-yl)-4-methylthiazole-5-sulfonamide (Compound 1)

A stirred solution of compound 1-4 (3.3 g, 7.096 mmol) in POCl₃ (33 mL) was allowed to stir at 90° C. for 5 h. The reaction mixture was concentrated under reduced pressure to dryness. The resulting residue obtained was dissolved in THF (66 mL), and aqueous ammonia (33 mL) was added at −5° C. and stirring continued at room temperature for another 12 h. After completion of the reaction, the reaction mixture was diluted with water and extracted with EtOAc. The combined organic layers were dried over anhydrous Na₂SO₄, filtered and concentrated under reduced pressure. The crude compound was purified by CombiFlash chromatography (eluting with 100% EtOAc) to afford the desired product Compound 1 (1.1 g, 44.64%) as a white solid. 1H NMR (400 MHz, DMSO-d₆) δ 7.65-7.59 (m, 2H), 7.55 (br s, 2H), 7.53-7.48 (m, 2H), 7.48-7.36 (m, 2H), 7.31-7.25 (m, 1H), 4.17 (t, J=6.1 Hz, 2H), 3.82 (t, J=5.6 Hz, 2H), 2.45 (s, 3H), 2.29-2.18 (m, 2H).

### Synthesis of 4′-bromo-2,5-difluoro-1,1′-biphenyl (Int. 1A)

To a stirred solution of 4-bromo-iodobenzene (5 g, 17.674 mmol) in 1,4 dioxane: H₂O (50:5 mL) were added (2,5-difluorophenyl) boronic acid (3.07 g, 19.441 mmol) and K₃PO₄ (7.5 g, 35.348 mmol) and the reaction mixture was purged under nitrogen for 10 min. To this resulting solution PdCl₂ (dppf) (1.29 g, 1.767 mmol) was added under nitrogen atmosphere. The reaction mixture was heated at 80° C. for 1 h. After completion of the reaction (monitored by TLC), the reaction mixture was cooled to room temperature, filtered through a pad of Celite® and washed with ethyl acetate. The filtrate was diluted with water and extracted with EtOAc. The combined organic layers were dried over anhydrous Na₂SO₄, filtered and concentrated under reduced pressure. The crude compound was purified by CombiFlash® chromatography (eluting with 100% heptane) to afford the title compound Int. 1A (2.3 g, 48.62%) as an off-white solid. TLC: 100% heptane (R_{f}: 0.5). 1H NMR (400 MHz, CDCl₃) δ 7.58 (d, J=8.3 Hz, 2H), 7.40 (d, J=7.3 Hz, 2H), 7.15-7.06 (m, 2H), 7.05-6.97 (m, 1H).

### Example 2: Biological Assay Data for 2-(3-(2′,5′-difluoro-[1,1′-biphenyl]-4-yl)-2-oxotetrahydropyrimidin-1(2H)-yl)-4-methylthiazole-5-sulfonamide (Compound 1)

### Cell Culture

Vero cells were cultured in Dulbecco's Modified Eagle Medium (DMEM) supplemented with 10% foetal bovine serum and 100 units/mL penicillin and streptomycin. The cells were passaged 2-3 times per week to maintain sub-confluent densities.

### HSV-1 Antiviral Assay

Vero cells were seeded into 96-well plates at a density of 2.5×10³ cells per well and allowed to attach overnight. Following attachment, the media was replaced with 50 μL of infection medium (DMEM supplemented with 2% foetal bovine serum and 100 units/mL penicillin and streptomycin). A Tecan D300e digital dispenser was then used to add compounds to the culture using an 8-point 3-fold serial dilution format. The DMSO concentration was normalized to 0.5% for all treatments. Following compound addition, 50 μL of infection medium containing 80 TCID50 HSV-1 was added to the cells and incubated at 37° C. for 4 days. After the incubation, the plates were equilibrated to room temperature, the media was removed, and 60 of a 1:1 dilution of Cell titer glow and phosphate buffered saline was added to the cells. Following a 5-minute incubation, cell viability was quantified by measuring luminance using a Tecan Infinite M1000 Pro plate reader.

### HSV-2 Antiviral Assay

Vero cells were seeded into 96-well plates at a density of 1.0×10⁴ cells per well and allowed to attach overnight. Following attachment, the media was replaced with 50 μL of infection medium (DMEM supplemented with 2% foetal bovine serum and 100 units/mL penicillin and streptomycin). A Tecan D300e digital dispenser was then used to add compounds to the culture using an 8-point 3-fold serial dilution format. The DMSO concentration was normalized to 0.5% for all treatments. Following compound addition, 50 μL of infection medium containing 160 TCID50 HSV-2 G strain was added to the cells and incubated at 37° C. for 5 days. After the incubation, 10 μL/well of WST-8 chromogenic reagent was added and the plates incubated at 37° C. for 3 hours. Following the incubation, cell viability was quantified by measuring the absorbance at 460 nm and 620 nm using a Tecan Infinite M1000 Pro plate reader.

In the HSV-1 antiviral assay, Compound 1 had an EC₅₀ of 0.019 μM (n=22). In the HSV-2 antiviral assay, Compound 1 had an EC₅₀ of 0.011 μM (n=35).

### Example 3: PK Studies in Rat, Monkey and Dog Following Intravenous Administration of Compound 1

### Formulation Preparation—Rat

A 0.2 mg/mL solution of Compound 1 in 10% NMP, 10% Solutol® HS15 and 80% Saline was prepared as follows:
1) A stock solution of Compound 1 in NMP was prepared.
2) An equal volume of the Compound 1 stock solution and Solutol® HS15 were combined and mixed.
3) The above solution was diluted with normal saline to reach the target concentration. The final formulation was a clear solution.

### Animal Dosing—Rat

Male sprague-dawley (SD) rats with 230-250 g body weight, ages 6-8 weeks, were used for the rat pharmacokinetic study. The animals had free access to food and water. The Compound 1 solution described above was administered via intravenous injection to animals (n=3) to achieve a 1.0 mg/kg dose.

At each time point, approximately 200 μL whole blood was collected in a K2EDTA tubes. Blood samples were put on ice and centrifuged at 2000 g for 5 min to obtain a plasma sample within 15 minutes. Plasma samples were stored at approximately −70° C. until analyzed by LC-MS according to the method described above.

### Results

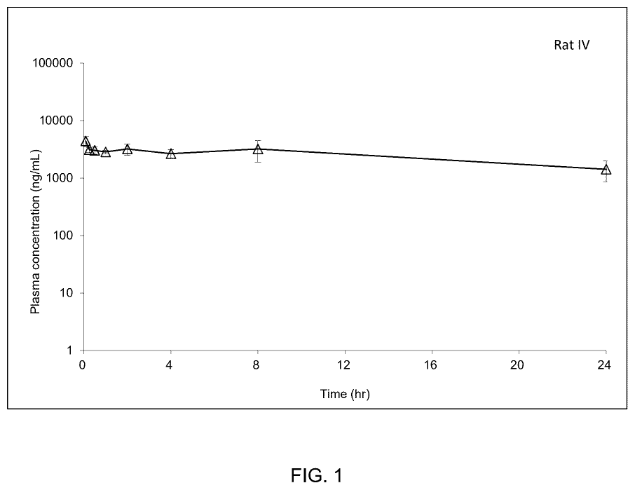

The mean plasma concentration profiles (n=3) after intravenous (IV) to rat at 1.0 mg/kg of Compound 1 solution can be found in FIG. 1.

### Formulation Preparation—Monkey

A 0.25 mg/mL solution of Compound 1 in 10% DMSO, 10% Solutol® HS15 and 80% Saline was prepared as follows:
1) A stock solution of Compound 1 in DMSO was prepared.
2) An equal volume of the Compound 1 stock solution and Solutol® HS15 were combined and mixed.
3) The above solution was diluted with normal saline to reach the target concentration. The final formulation was a clear solution.

### Animal Dosing—Monkey

Male non-naïve Cyno Monkeys with were used for the monkey PK study. The animals had free access to food and water. The prepared solution was administered via intravenous injection to each animal group (n=3) to achieve a 0.25 mg/kg dose.

At each time point, approximately 0.5 mL whole blood was collected into K2EDTA tubes. Blood samples were put on to ice and centrifuged at 2000 g for 5 min to obtain plasma sample within 15 minutes. Plasma samples were stored at approximately −70° C. until analyzed by LC-MS.

### Results

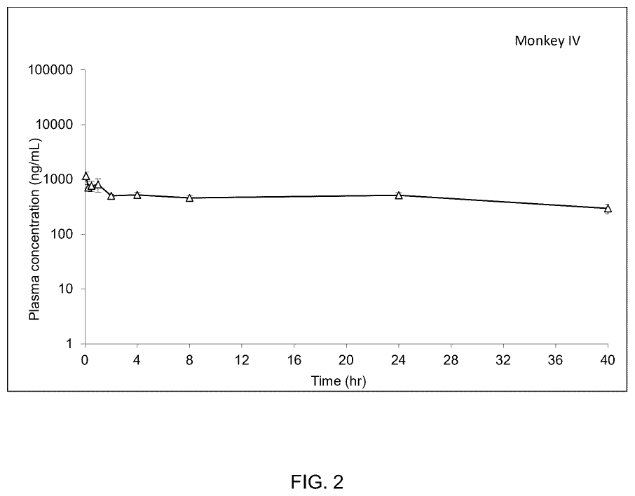

The mean plasma concentration profiles (n=3) after intravenous (IV) at 0.25 mg/kg of Compound 1 solution can be found in FIG. 2.

### Formulation Preparation—Dog

A 0.15 mg/mL solution of Compound 1 in 10% DMSO, 10% Solutol® HS15 and 80% Saline was prepared as follows:
1) A stock solution of Compound 1 in DMSO was prepared.
2) An equal volume of the Compound 1 stock solution and Solutol® HS15 were combined and mixed.
3) The above solution was diluted with normal saline to reach the target concentration. The final formulation was a clear solution.

### Animal Dosing—Dog

Male non-naïve beagles were used for the dog PK study. The animals had free access to food and water. The prepared solution was administered via intravenous injection to each animal group (n=3) to achieve a 0.15 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

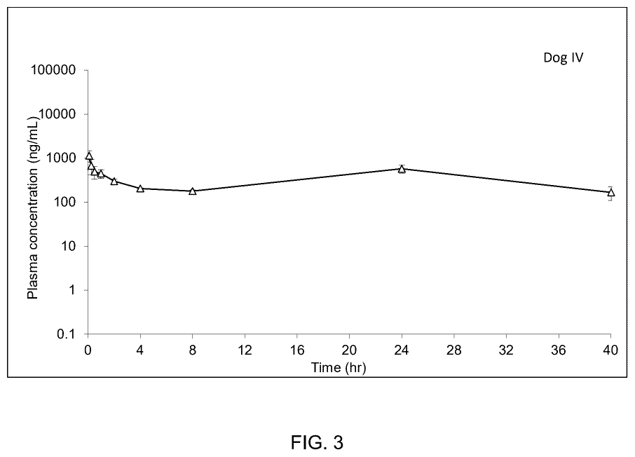

The mean plasma concentration profiles (n=3) after intravenous (IV) at 0.15 mg/kg of Compound 1 solution can be found in FIG. 3.

### Summary

IV dosing of Compound 1 in rat, monkey and dog at doses of 1 mg/kg, 0.25 mg/kg and 0.15 mg/kg respectively gave the long terminal half-lives as shown in Table 15. Based on this data, it is expected that Compound 1 will also exhibit a long terminal half-life in human.

TABLE 15
Half-life data from IV studies

| | Compound 1 |
|---|---|
| Rat | 22 |
| t_{1/2} (hr) | |
| Monkey | 49.6 |
| t_{1/2} (hr) | |
| Dog | 34.9 |
| t_{1/2} (hr) | |

### Example 3A: Further Compound 1 PK Studies in Rat, Monkey, Dog and Mini-Pig Following IV Administration

### Formulation Preparation—Rat

A 0.2 mg/mL solution of Compound 1 in 10% NMP, 10% Solutol HS15 and 80% Saline was prepared as follows:
1) A stock solution of Compound 1 in NMP was prepared.
2) An equal volume of the Compound 1 stock solution and Solutol HS15 were combined and mixed.
3) The above solution was diluted with normal saline to reach the target concentration. The final formulation was a clear solution.

### Animal Dosing—Rat

Male Sprague Dawley (SD) rats with 180-184 g body weight, ages 6-8 weeks, were used for the rat pharmacokinetic study. The animals had free access to food and water. The Compound 1 solution described above was administered via intravenous injection (1 mL/kg) to each animal (n=3) to achieve a 0.2 mg/kg dose.

At each time point, approximately 200 μL whole blood was collected in a K2EDTA tubes. Blood samples were put on ice and centrifuged at 2000 g for 5 min to obtain a plasma sample within 15 minutes. Plasma samples were stored at approximately −70° C. until analyzed by LC-MS according to the method described above.

### Formulation Preparation—Monkey

A 0.2 mg/mL solution of Compound 1 in 10% DMSO, 10% Solutol HS15 and 80% Saline was prepared as follows:
1) A stock solution of Compound 1 in DMSO was prepared.
2) An equal volume of the Compound 1 stock solution and Solutol HS15 were combined and mixed.
3) The above solution was diluted with normal saline to reach the target concentration. The final formulation was a clear solution.

### Animal Dosing—Monkey

Male non-naïve Cyno Monkeys with were used for the monkey PK study. The animals had free access to food and water. The Compound 1 solution described above was administered via intravenous injection (1 mL/kg) to each animal (n=3) to achieve a 0.2 mg/kg dose.

At each time point, approximately 0.5 mL whole blood was collected into K2EDTA tubes. Blood samples were put on to ice and centrifuged at 2000 g for 5 min to obtain plasma sample within 15 minutes. Plasma samples were stored at approximately −70° C. until analyzed by LC-MS.

### Formulation Preparation—Dog

A 0.15 mg/ml solution of Compound 1 in 10% DMSO, 10% Solutol HS15 and 80% Saline was prepared as follows:
1) A stock solution of Compound 1 in DMSO was prepared.
2) An equal volume of the Compound 1 stock solution and Solutol HS15 were combined and mixed.
3) The above solution was diluted with normal saline to reach the target concentration. The final formulation was a clear solution.

### Animal Dosing—Dog

Male non-naïve beagles were used for the dog PK study. The animals had free access to food and water. The Compound 1 solution described above was administered via intravenous injection (1 mL/kg) to each animal (n=3) to achieve a 0.15 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Formulation Preparation—Minipig

A 0.25 mg/mL solution of Compound 1 in 10% DMSO, 10% Solutol HS15 and 80% Saline was prepared as follows:
1) A stock solution of Compound 1 in DMSO was prepared.
2) An equal volume of the Compound 1 stock solution and Solutol HS15 were combined and mixed.
3) The above solution was diluted with normal saline to reach the target concentration. The final formulation was a clear solution.

### Animal Dosing—Minipig

Naïve Bama Pigs (15-16 kg) were used for the minipig PK study. The animals had free access to food and water. The Compound 1 solution described above was administered via intravenous injection (1 mL/kg) to each animal (n=3) to achieve a 0.25 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

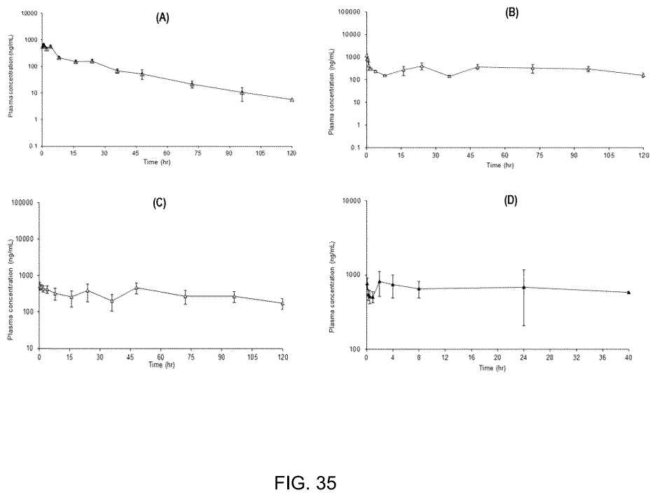

The mean plasma concentration profiles (n=3) after intravenous (IV) injection of Compound 1 solution in rat, dog, monkey and mini-pig can be found in FIG. 35 (A)-(D).

### Summary

IV dosing of Compound 1 in rat, dog, monkey and mini-pig at doses of 0.2 mg/kg, 0.15 mg/kg, 0.2 mg/kg and 0.25 mg/kg respectively gave the terminal half-lives and clearances as shown in the following table:

| | Half-life | Clearance |
|---|---|---|
| Species | (days) | (L/hr/kg) |
| Rat | 0.8 | 0.02 |
| Dog | 2.3 | 0.002 |
| Monkey | 3.0 | 0.004 |
| Mini-pig | 5.6 | 0.0018 |

Based on the above PK data in multiple species, and using allometric scaling, the predicted human biological terminal half-life of Compound 1 is 7.6 days (182 hours) with a clearance of 0.06 L/hr.

### Example 4: Preparation of Compound 1 Solid Dispersions

A preliminary screen for solid dispersions prepared by rotary evaporation was conducted. Compound 1 (30 mg), polymer and surfactant were dissolved in THF/Acetone/H₂O (5/4/1, v/v/v) to obtain a clear solution. These solutions were placed under 60° C. in a rotary evaporator to complete drying. After drying, the solid dispersions were analyzed to determine crystallinity. From this preliminary screen, a solid dispersion using HPMCAS LG was selected for manufacture using a spray drying method. Four lots of solid dispersion were prepared using the conditions described in Table 16.

TABLE 16
Spray drying conditions

| | |
|---|---|
| SD composition | 15:85 Compound 1:HPMCAS LG |
| Spray solvent | 5:4:1 THF:Acetone:Water |
| Spray Solution (wt % total solids) | 2.2-2.7 |
| Spray Dryer | Yamato DL410 |
| Nozzle Type | Binary Nozzle 1A |
| Solution Flow Rate (kg/hr) | 45-50 ml/min |
| Atomization Pressure (bar) | 0.1 |
| Inlet Temperature (° C.) | 100 |
| Outlet Temperature (° C.) | 50 |
| Secondary Drying Temperature/ | 24-48 hours at 40° C. |
| Time | |

The four lots of solid dispersion were characterised for drug loading, purity, residual solvents, water content and DSC and results from this characterisation work are shown in Table 17.

TABLE 17
Solid dispersion characterisation

| | For- | | | | |
|---|---|---|---|---|---|
| Ex. | mulations | Assay | Purity | Residual solvents | DSC |
| Lot 1 | 15:85 | 13.6% | 97.9% | Water content: | Tg~105.51 |
| | Compound | | | 3.2% | |
| | 1: | | | Solvent residue: | |
| | HPMCAS | | | THF 93 ppm | |
| | LG | | | | |
| Lot 2 | 15:85 | 14.2% | 98.3% | Water content: | Tg~94.63 |
| | Compound | | | 0.61% | |
| | 1: | | | Solvent residue: | |
| | HPMCAS | | | Acetone 1028 ppm; | |
| | LG | | | THF 5765 ppm | |
| Lot 3 | 15:85 | 13.7% | 98.5% | Water content: | Tg~107.76 |
| | Compound | | | 1.50% | |
| | 1: | | | Solvent residue: | |
| | HPMCAS | | | Acetone 322 ppm; | |
| | LG | | | THF 7537 ppm | |
| Lot 4 | 15:85 | 14.1% | 99.1% | Water content: | Tg~105.90 |
| | Compound | | | 1.47% | |
| | 1: | | | Solvent residue: | |
| | HPMCAS | | | Acetone 853 ppm; | |
| | LG | | | THF 10824 ppm | |

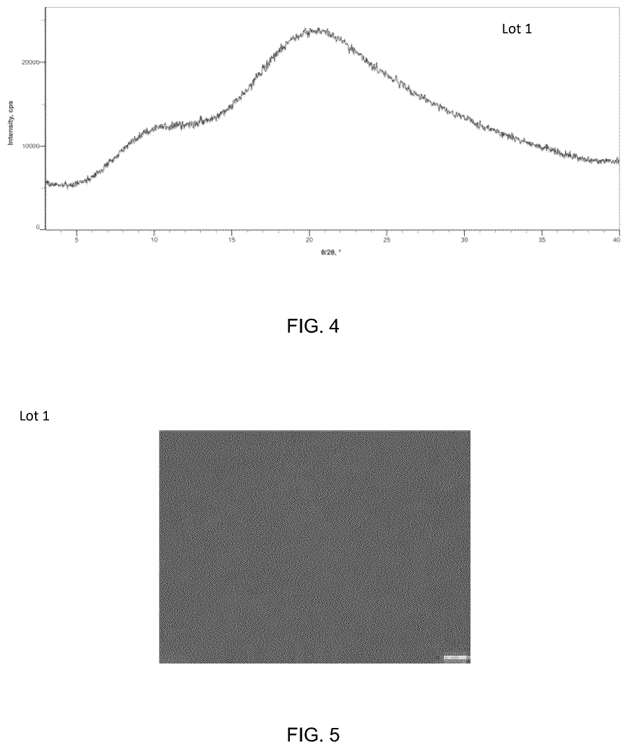

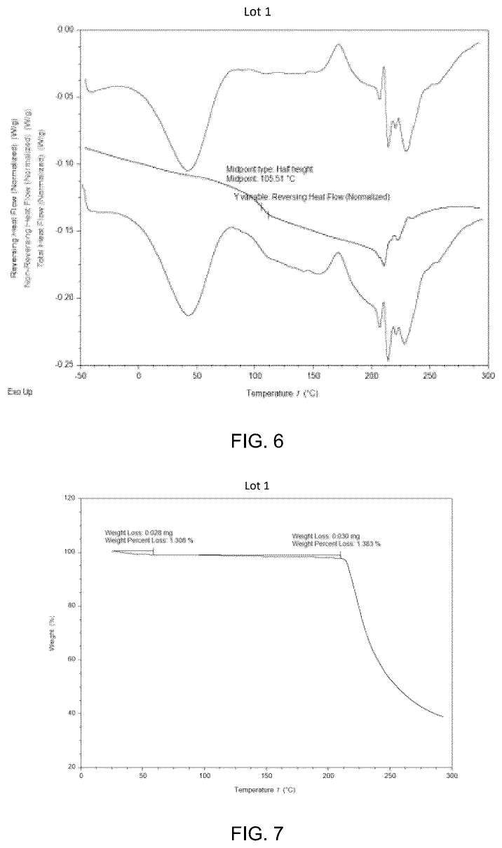

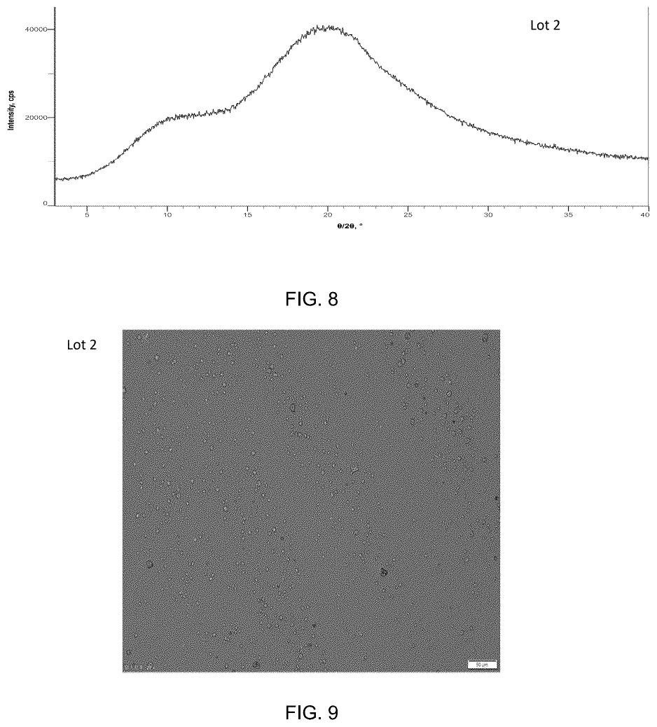

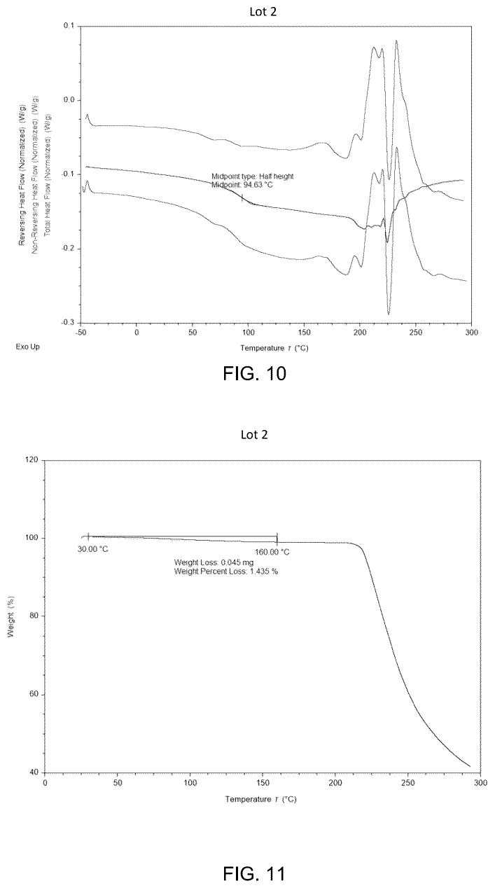

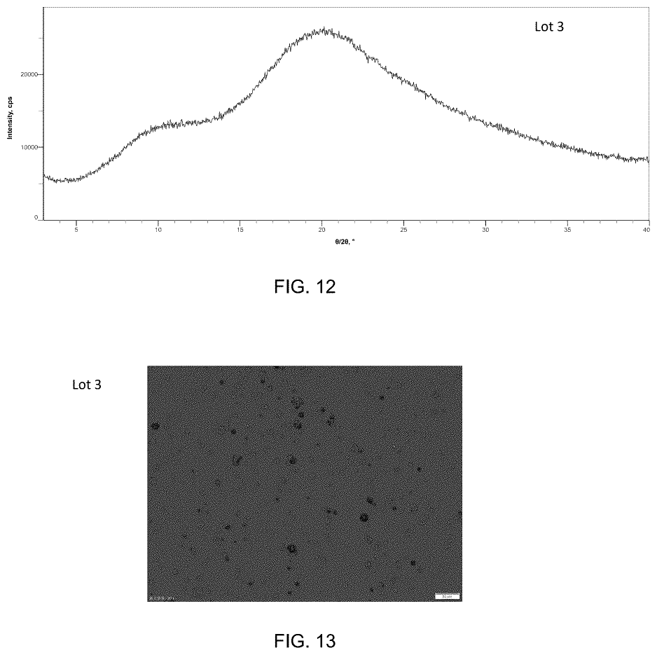

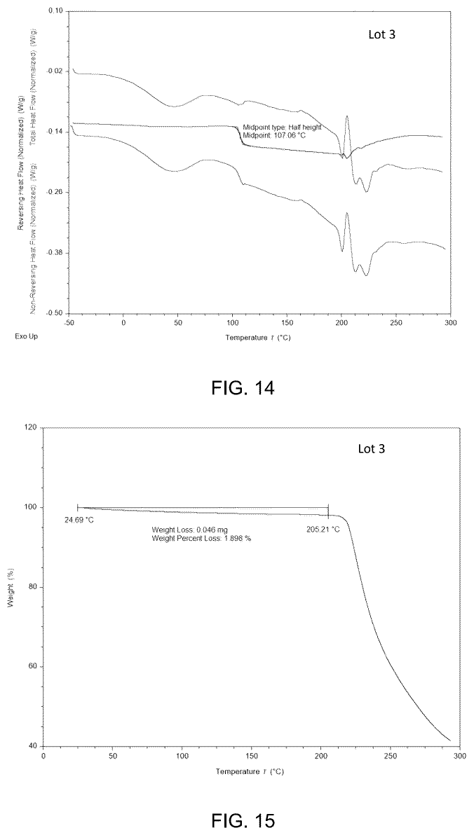

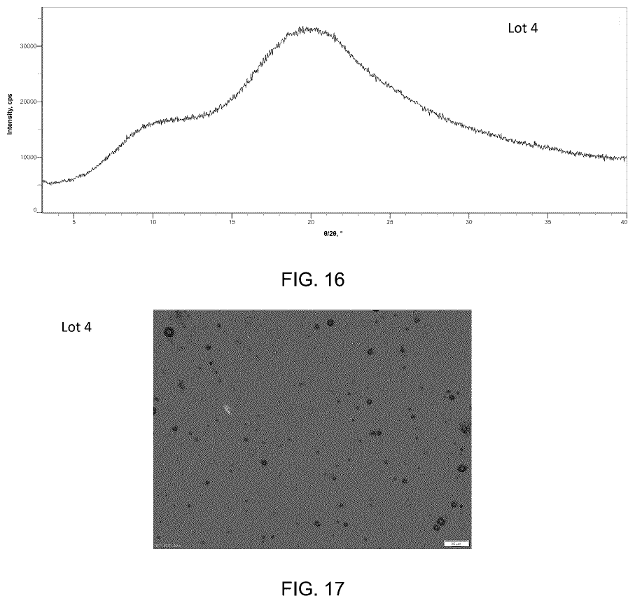

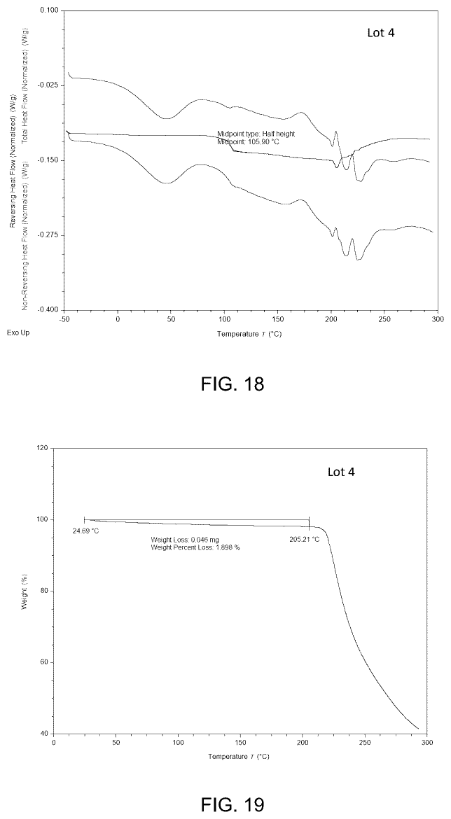

Initial characterization by XRPD indicated that all the SDs were amorphous dispersions and no crystalline peaks were observed in the SD diffractograms (FIGS. 4, 8, 12 and 16). Thermal analysis showed that all dispersions had a single T_{g} indicating an intimately mixed amorphous solid dispersion with good homogeneity (see FIGS. 6, 10, 14 and 18). PLM images are shown in FIGS. 5, 9, 13 and 17 and TGA profiles are shown in FIGS. 7, 11, 15 and 19.

### Example 5: Single Dose Oral Compound 1 PK Study in Rat

### Formulation Preparation: Solution in 90% NMP/10% TPGS

A solution of Compound 1 in 90% NMP/10% Vitamin E TPGS at a concentration of 100 mg/mL was prepared as follows:
1) Weighed appropriate amount of Compound 1
2) Dissolved Compound 1 in 90% NMP/10% Vitamin E TPGS solvent.

The final dosing formulation was a clear solution.

### Formulation Preparation: Nanosuspension

A 0.5% PVP K30 plus 0.5% Poloxamer 188 suspension at a concentration of 50 mg/ml was prepared as follows:
1) Weighed appropriate amount of Compound 1
2) Dissolved PVP K30 and Poloxamer 188 in water to prepare vehicle
3) Added drug into the vehicle and nano-milled

The final dosing formulation was a white homogenous suspension. Particle size distribution (PSD) of the suspension was determined as DV₁₀=0.144 μm, DV₅₀=0.200 μm and DV₉₀=0.303 μm.

### Formulation Preparation: Spray Dried Dispersion (SDD) Suspension

A SDD (Lot 1 from Example 4, Table 17) in 0.5% HPMC in Water at a concentration of 100 mg/mL (drug concentration of 13.6 mg/ml) was prepared as follows:
1) 3521.31 mg SDD was weighed into an appropriate sized mortar.
2) 5 mL of HPMC solution was added slowly. While adding, pestle was used to intimately mix and wet the powder to get a wet paste
3) 30.213 mL 0.5% HPMC in water was added slowly and the pestle was used to break up powder agglomeration to achieve a suspension
4) Suspension was transferred to an appropriate vessel.

The final dosing formulation was a white homogenous suspension. The suspension was stirred prior to dosing.

### Animal Dosing

Male sprague-dawley (SD) rats with 230-250 g body weight, ages 6-8 weeks were used for the rat PK study. The animals had free access to food and water. The prepared compositions were administered via oral administration to each animal group (n=3) with appropriate dosing volume to achieve the respective doses.

At each time point, approximately 110 μL whole blood/time point was collected in K2EDTA tube via jugular vein. Blood sample was put on ice and centrifuged at 2000 g for 5 min to obtain plasma sample within 15 minutes. Plasma samples were stored at approximately −70° C. until analyzed by LC-MS.

### Results

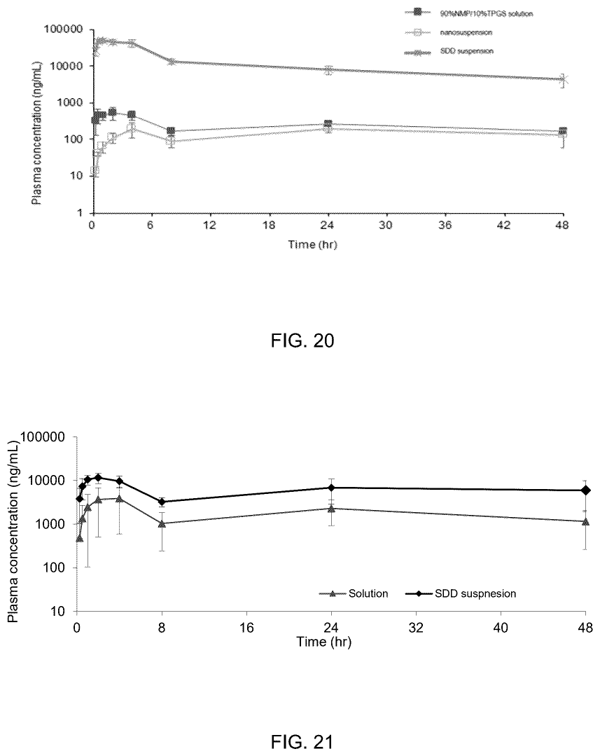

The dosing amounts and resulting PK parameters after oral administration of the Compound 1 solution in 90% NMP/10% TPGS, the Compound 1 nanosuspension and the Compound 1 SDD suspension are shown in Table 18 and the mean plasma concentration profiles (n=3) are shown in FIG. 20. The Compound 1 SDD suspension achieved much higher exposure than the solution and nanosuspension formulations. Furthermore, high levels and prolonged exposure out to at least 48 hours (length of the study) was achieved.

TABLE 18
Rat dosing and PK Data

| | Drug | Vol. | Dose | Cₘₐₓ | AUCₗₐₛₜ |
|---|---|---|---|---|---|
| | conc. | (mL/ | (mL/ | (ng/ | (hr* |
| Formulation | (mg/mL) | kg) | kg) | mL) | ng/ml) |
| 90% NMP/10% TPGS solution | 100 | 1 | 100 | 536 | 11362 |
| 0.5% PVP K30 plus 0.5% | 50 | 2 | 100 | 211 | 7067 |
| Poloxamer 188 | | | | | |
| nanosuspension | | | | | |
| SDD in 0.5 % HPMC in water | 13.6 | 7.35 | 100 | 49667 | 588287 |
| suspension | | | | | |

### Example 6: Single Dose Oral Compound 1 PK Study in Dog

### Formulation Preparation: Solution in 85% PEG300/10% TPGS/5% EtOH

A solution of Compound 1 in 85% PEG300/10% TPGS/5% EtOH at a concentration of 2 mg/mL was prepared as follows:
1) Weighed 177.92 mg Compound 1 into a clear tube
2) Added 87.759 mL of 85% PEG300/10% TPGS/5% EtOH into the tube containing the compound
3) Vortex the tube for 5 min. Sonicate it for 10 min.

The final dosing formulation was a colourless clear solution.

### Formulation Preparation: SDD Suspension

A SDD (Lot 1 from Example 4, Table 17) in 0.5% HPMC in Water at 100 mg/ml (drug concentration of 13.6 mg/ml) was prepared as follows:
1) 3521.31 mg SDD was weighed into an appropriate sized mortar.
2) 5 mL of HPMC solution was added slowly. While adding, pestle was used to intimately mix and wet the powder to get a wet paste
3) 30.213 mL 0.5% HPMC in water was added slowly and the pestle was used to break up powder agglomeration to achieve a suspension
4) Suspension was transferred to an appropriate vessel.

The final dosing formulation was a white homogenous suspension. The suspension was stirred prior to dosing.

### Animal Dosing

Male non-naïve beagles were used for the dog PK study. The animals had free access to food and water. The prepared solution and suspension formulations were administered via oral administration to each animal group (n=3) with appropriate dosing volume to achieve the respective doses.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

The dosing amounts and resulting PK parameters after oral administration of the Compound 1 solution in 85% PEG300/10% TPGS/5% EtOH and Compound 1 SDD suspension are shown in Table 19 and the mean plasma concentration profiles (n=3) are shown in FIG. 21. The Compound 1 SDD suspension achieved superior exposure than the solution formulation. High plasma levels were achieved and prolonged exposure out to at least 48 hours (length of the study) was evident. Furthermore, PK variability in plasma levels for the SDD suspension was significantly lower than that seen with the Compound 1 in 85% PEG300/10% TPGS/5% EtOH solution composition.

TABLE 19
Dog dosing and PK Data

| | Dose | Cₘₐₓ | AUCₗₐₛₜ | Cₘₐₓ/ | AUCₗₐₛₜ / |
|---|---|---|---|---|---|
| Formulation | (mg/kg) | (ng/ml) | (hr*ng/ml) | Dose | Dose |
| SDD in 0.5% HPMC | 10 | 11607 | 294851 | 1161 | 29485 |
| in water suspension | | | | | |
| Solution in 85% PEG300/ | 4 | 3673 | 86767 | 918 | 21692 |
| 10% TPGS/5% EtOH | | | | | |

### Example 7: Seven-Day Oral Dosing PK Study in Rat

Formulation 1: SDD Suspension at Compound 1 Concentration of 1 mg/mL, Dosing at 10 mL/Kg to Achieve 10 mg/Kg

A SDD (Lot 4 from Example 4, Table 17) in 0.5% HPMC in Water at 1 mg mg/mL was prepared as follows:
1) 485.06 mg SDD was weighed into a new tube
2) 68.393 mL of 0.5% HPMC in water was added into the tube containing the SDD
3) Mixture was stirred for 40 mins
4) Mixture was homogenised with ULTRA-TURRAX T10 on medium speed for about 1 min and then mixing was pauses for half minute. This procedure was repeated 4 times.

The final dosing formulation was a white homogenous suspension. The suspension was stirred prior to dosing.

Formulation 2: SDD Suspension at Compound 1 Concentration of 10 mg/mL, Dosing at 10 mL/Kg to Achieve 100 mg/Kg

A SDD (Lot 4 from Example 4, Table 17) in 0.5% HPMC in Water at 10 mg mg/mL was prepared as follows:
1) 4822.98 mg SDD was weighed into a new tube
2) 68.004 mL of 0.5% HPMC in water was added into the tube containing the SDD
3) Mixture was stirred for 40 mins
4) Mixture was homogenised with ULTRA-TURRAX T10 on medium speed for about 1 min and then mixing was pauses for half minute. This procedure was repeated 4 times.

The final dosing formulation was a white homogenous suspension. The suspension was stirred prior to dosing.

Formulation 3: SDD Suspension at Compound 1 Concentration of 30 mg/mL, Dosing at 10 mL/Kg to Achieve 300 mg/Kg

A SDD (Lot 4 from Example 4, Table 17) in 0.5% HPMC in Water at 30 mg mg/ml was prepared as follows:
1) 14251.25 mg SDD was weighed into a new tube
2) 66.981 mL of 0.5% HPMC in water was added into the tube containing the SDD
3) Mixture was stirred for 40 mins
4) Mixture was homogenised with ULTRA-TURRAX T10 on medium speed for about 1 min and then mixing was pauses for half minute. This procedure was repeated 4 times.

The final dosing formulation was a white homogenous suspension. The suspension was stirred prior to dosing.

### Animal Dosing

Male sprague-dawley (SD) rats with 230-250 g body weight, ages 6-8 weeks were used for the rat PK study. The animals had free access to food and water. The prepared compositions were administered via oral administration daily for seven days to each animal group (n=3) with appropriate dosing volume to achieve the respective doses.

At each time point, approximately 110 μL whole blood/time point was collected in K2EDTA tube via jugular vein. Blood sample was put on ice and centrifuged at 2000 g for 5 min to obtain plasma sample within 15 minutes. Plasma samples were stored at approximately −70° C. until analyzed by LC-MS.

### Results

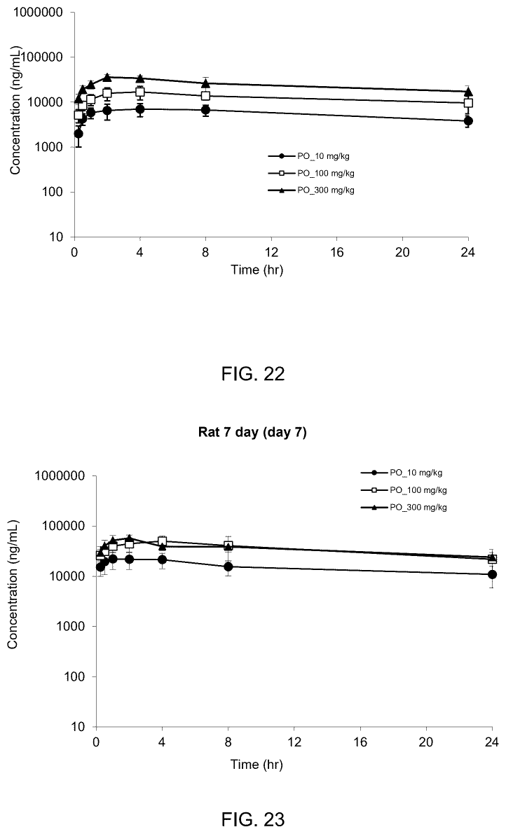

The mean plasma concentration profiles (n=3) of the compositions at the three dosing levels at Day 1 and Day 7 are shown in FIGS. 22 and 23. The Compound 1 SDD suspension was able to rapidly achieve high exposure. Evidence of accumulation was present at Day 7 and the plasma levels of the 100 mg/kg and 300 mg/kg doses were similar.

### Example 8: Seven-Day Oral Dosing PK Study in Dog

Formulation 1: SDD Suspension at Compound 1 Concentration of 0.2 mg/mL, Dosing at 5 mL/Kg to Achieve 1 mg/Kg

A SDD (Lot 2 from Example 4, Table 17) in 0.5% HPMC in Water at 0.2 mg mg/mL was prepared as follows:
1) 338.14 mg SDD was weighed into appropriately sized mortar
2) 5 mL of HPMC solution was added slowly. While adding, pestle was used to intimately mix and wet the powder to get a wet paste
3) 229.435 mL 0.5% HPMC in water was added slowly and the pestle was used to break up powder agglomeration to achieve a suspension
4) Suspension was transferred to an appropriate vessel.

The final dosing formulation was a white homogenous suspension. The suspension was stirred prior to dosing.

Formulation 2: SDD Suspension at Compound 1 Concentration of 2 mg/mL, Dosing at 5 mL/Kg to Achieve 10 mg/Kg

A SDD (Lot 2 from Example 4, Table 17) in 0.5% HPMC in Water at 2 mg mg/mL was prepared as follows:
1) 3378.05 mg SDD was weighed into appropriately sized mortar
2) 5 mL of HPMC solution was added slowly. While adding, pestle was used to intimately mix and wet the powder to get a wet paste
3) 227.707 mL 0.5% HPMC in water was added slowly and the pestle was used to break up powder agglomeration to achieve a suspension
4) Suspension was transferred to an appropriate vessel.

The final dosing formulation was a white homogenous suspension. The suspension was stirred prior to dosing.

Formulation 3: SDD Suspension at Compound 1 Concentration of 20 mg/mL, Dosing at 5 mL/Kg to Achieve 100 mg/Kg

A SDD (Lot 2 from Example 4, Table 17) in 0.5% HPMC in Water at 20 mg mg/mL was prepared as follows:
1) 33.858 g SDD was weighed into appropriately sized mortar
2) 45 mL of HPMC solution was added slowly. While adding, pestle was used to intimately mix and wet the powder to get a wet paste
3) 185.234 mL 0.5% HPMC in water was added slowly and the pestle was used to break up powder agglomeration to achieve a suspension
4) Suspension was transferred to an appropriate vessel.

The final dosing formulation was a white homogenous suspension. The suspension was stirred prior to dosing.

### Animal Dosing

Male non-naïve beagles were used for the dog PK study. The animals had free access to food and water. The prepared compositions were administered via oral administration daily for seven days to each animal group (n=3) with appropriate dosing volume to achieve the respective doses.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

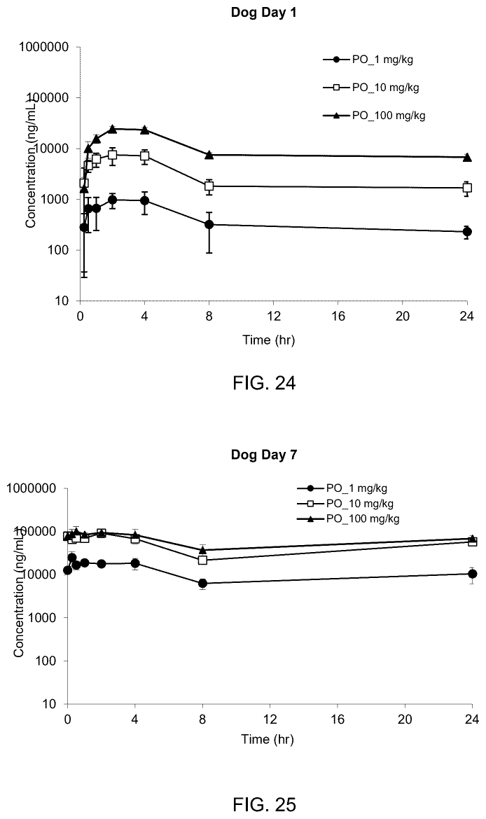

The mean plasma concentration profiles (n=3) of the compositions at the three dosing levels at Day 1 and Day 7 are shown in FIGS. 24 and 25. The Compound 1 SDD suspension was able to rapidly achieve high exposure. Evidence of accumulation was present at Day 7 and the plasma levels of the 10 mg/kg and 100 mg/kg doses were similar.

### Example 9: Fourteen-Day Oral Dosing PK Study in Dog

Formulation 1: SDD Suspension at Compound 1 Concentration of 3 mg/mL, Dosing at 5 mL/Kg to Achieve 15 mg/Kg

A SDD (Lot 3 from Example 4, Table 17) in 0.5% HPMC in Water at 0.2 mg mg/mL was prepared as follows:
1) 4851.0 mg SDD was weighed into appropriately sized mortar
2) 5 mL of HPMC solution was added slowly. While adding, pestle was used to intimately mix and wet the powder to get a wet paste
3) 214.912 mL 0.5% HPMC in water was added slowly and the pestle was used to break up powder agglomeration to achieve a suspension
4) Suspension was transferred to an appropriate vessel.

The final dosing formulation was a white homogenous suspension. The suspension was stirred prior to dosing.

### Animal Dosing

Male or female non-naïve beagles were used for the dog PK study. The animals had free access to food and water. The prepared compositions were administered via oral administration daily for fourteen days to each animal group (n=2) with a dosing volume of 5 ml/kg to achieve 15 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

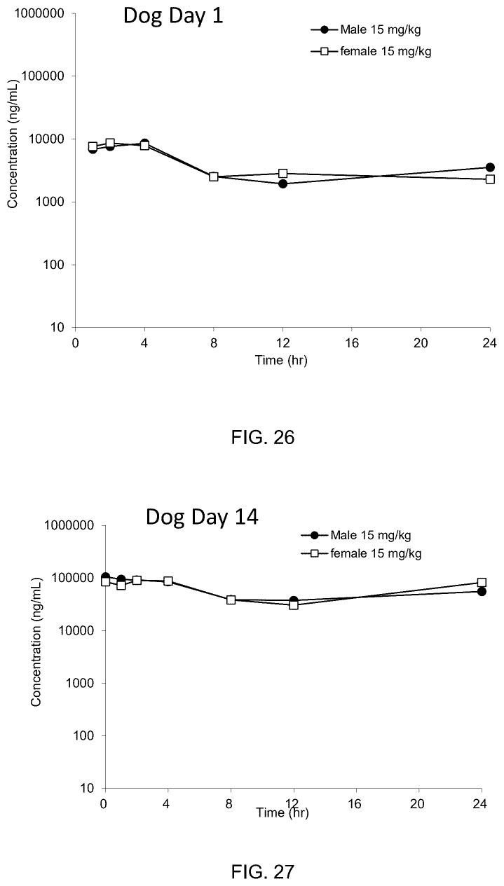

The mean plasma concentration profiles (n=2) of the compositions in male and female dogs at Day 1 and Day 14 are shown in FIGS. 26 and 27. The Compound 1 SDD suspension was able to rapidly achieve high exposure. Evidence of accumulation was present at Day 14. Plasma levels for the female and male dogs were similar.

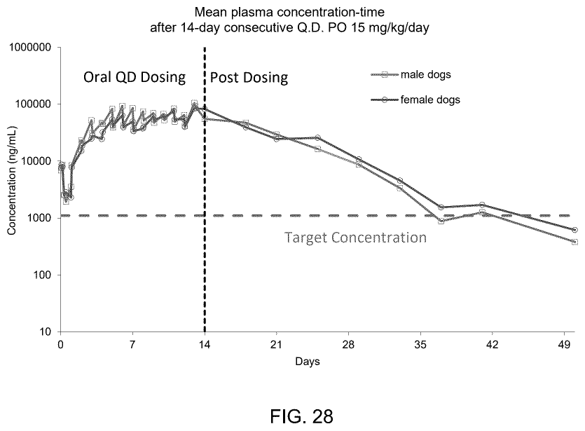

The mean plasma concentration-time profile after 14-days of consecutive once daily oral dosing at 15 mg/kg/day (n=2 for each sex group) is shown in FIG. 28. The Compound 1 SDD suspension was able to rapidly achieve high exposure. Remarkably, plasma concentrations remained very high even after dosing was stopped at 14 days. Plasma levels remained significantly higher than a target level of 1100 ng/ml for a prolonged period.

### Example 10: Dog IV Study with and without Oral Charcoal Administration

### Formulation Preparation—IV

A 0.1 mg/mL solution of Compound 1 in 10% DMSO/10% Solutol HS15/80% Saline was prepared as follows:
1) 4.63 mg Compound 1 was weighed into appropriately sized tube.
2) 4.57 mL of DMSO was added into the tube. Vortexed for 2 minutes.
3) 4.57 mL of Solutol HS15 was added into above tube. Vortexed for 2 minutes.
4) 36.54 mL of saline was added. Vortex for 1 minute.

The final formulation was a clear solution.

### Formulation Preparation—Oral Activated Charcoal

A 100 mg/mL suspension of Activated Charcoal in Saline was prepared as follows:
1) 300 g Activated Charcoal was weighed into appropriately sized tube.
2) 3000 mL of saline was added into the tube.
3) Mixture was stirred for 10 minutes, and then sonicated for 10 minutes.

The final formulation was a suspension.

### Animal Dosing—Dog

Two groups of Male non-naïve beagles were used for the dog PK study (n=2 per group). The prepared oral formulation was administered via oral administration with 25 gram dose of activated charcoal to Group 2 animals. Immediately after the PO administration, the prepared IV formulation was administered via 10 minutes IV infusion to both Group 1 and Group 2 animals with appropriate dosing volume to achieve the respective doses. Group 1 animals were fed before administration. Group 2 animals were fasted overnight before administration and were fed at 4 hours post oral administration.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −80° C. until analyzed by LC-MS.

### Results

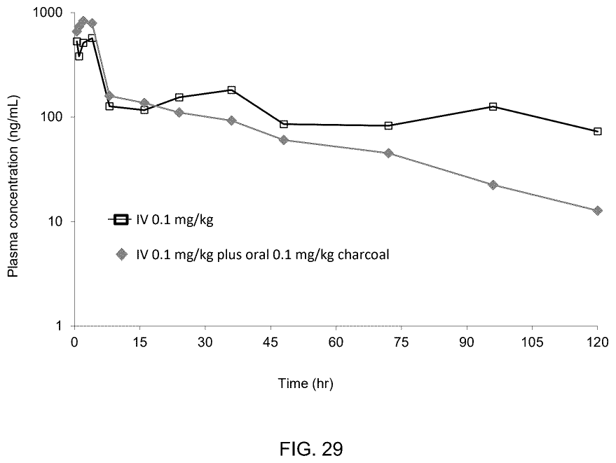

The mean plasma concentration profiles (n=3) after intravenous (IV) administration at 0.10 mg/kg of Compound 1 with and without oral charcoal administration can be found in FIG. 29. It can been seen that clearance was slower and overall exposure was higher when Compound 1 was administered without oral charcoal administration. While with co-administration of charcoal orally, Compound 1 could be adsorbed by charcoal in intestine and reabsorption was reduced indicating that intestinal reabsorption may play a part in the clearance and exposure upon dosing of Compound 1.

Summary from PK Studies

In 2 different species, long acting oral formulations containing the helicase primase inhibitor, Compound 1, have been found to provide rapid exposure and continuous and steady plasma levels of the drug for significantly prolonged periods of time following oral administration. Furthermore, release has been achieved at different dosing levels and variability in PK has been low. Intestinal reabsorption may play a part in the high exposure achieved and clearance upon oral dosing of Compound 1.

### Example 11: Preparation of Further Compound 1 Solid Dispersion

A solid dispersion of Compound 1 using HPMCAS HG was manufactured using a spray drying method. Preparation conditions are described in Table 20.

TABLE 20
Spray drying conditions

| | |
|---|---|
| SD composition | 20:80 Compound 1:HPMCAS HG |
| Spray solvent | 95:5 THF:Water |
| Spray Solution (wt % total solids) | 2 |
| Spray Dryer | Buchi B-290 |
| Nozzle Type | Two-Fluid −1.5 mm Air Cap, 0.7 mm |
| | Liquid Tip |
| Solution Flow Rate (mL/min) | 12.2 |
| Inlet Temperature (° C.) | 120-121 |
| Outlet Temperature (° C.) | 62-67 |
| Secondary Drying Temperature/ | 89 hours at 40° C. |
| Time | |

The solid dispersion was characterised for drug loading and purity and the results from this characterisation work are shown in Table 21.

TABLE 21
Solid dispersion characterisation

| Ex. | Formulations | Assay | Purity |
|---|---|---|---|
| Lot 1 | 20:80 Compound 1: | 20.1% | 99.7% |
| | HPMCAS HG | | |

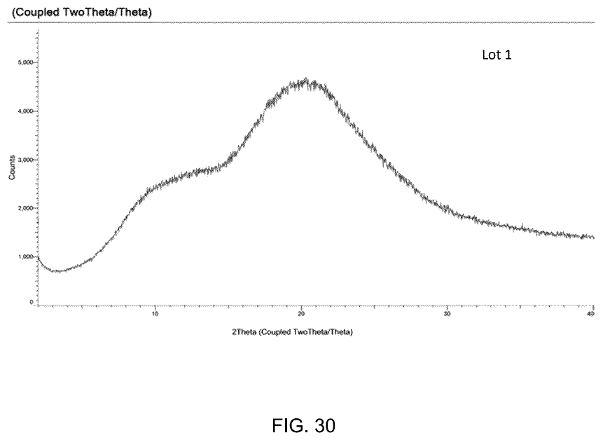

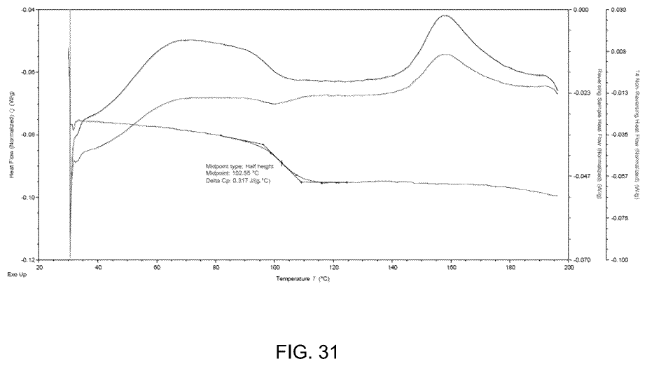

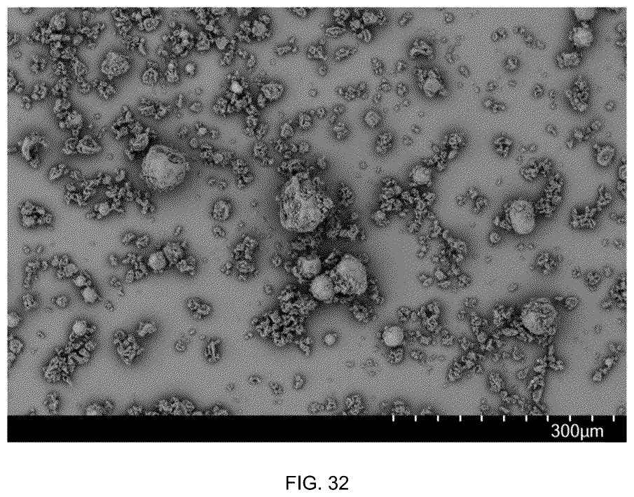

Initial characterization by XRPD indicated that the SDD was amorphous and no crystalline peaks were observed in the SD diffractogram (FIG. 30). Thermal analysis showed that the dispersion had a single T_{g} indicating an intimately mixed amorphous solid dispersion with good homogeneity (see FIG. 31). An SEM image is shown in FIG. 32.

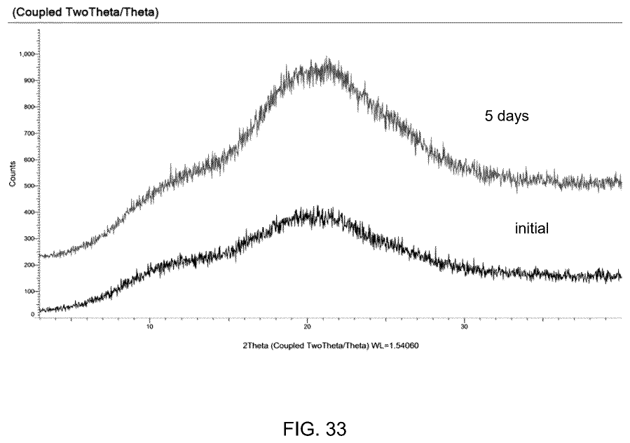

A stability study was also conducted. The HPMCAS HG SDD was stressed at 40° C./75% RH Open conditions for 5 days, and the SDD was found to be physically stable. No crystalline peaks were observed. Also, the SDD was chemically stable and no additional impurities or increase of impurities were observed (see Table 22 and FIG. 33).

TABLE 22
Solid dispersion stability characterisation

| | | | Purity | Area % at RRT | | | |
|---|---|---|---|---|---|---|---|
| Conditions | Appearance | XRPD | (%) | 0.56 | 0.80 | 1.36 | 1.72 |
| Initial | white | Amorphous | 99.61 | 0.11 | 0.10 | 0.07 | 0.11 |
| | powder | | | | | | |
| 40 C./75%/ | white | Amorphous | 99.61 | 0.11 | 0.10 | 0.07 | 0.11 |
| open, 5 d | powder | | | | | | |

### Example 12: Single Dose Oral Compound 1 PK Study in Dog Under Fed and Fasted Conditions

### Formulation Preparation: SDD Suspension:

A SDD (Lot 1 from Example 11, Table 20 and 21) in 0.5% HPMC in water at 25 mg/mL (drug concentration of 5 mg/mL) was prepared as follows:
5) 3012 mg SDD was weighed into an appropriate sized mortar.
6) 5 mL of 0.5% HPMC in water was added slowly. While adding, pestle was used to intimately mix and wet the powder to get a wet paste
7) 115.5 mL 0.5% HPMC in water was added slowly and the pestle was used to break up powder agglomeration to achieve a homogeneous suspension
8) Suspension was transferred to an appropriate vessel.

The final dosing formulation was a white homogenous suspension. The suspension was stirred prior to dosing.

### Animal Dosing

Male non-naïve beagles were used for the dog PK study and split into two groups. In Group 1, the animals were fasted overnight before dosing, then had free access to food and water at 4 hours post dosing. In Group 2, all animals were fed prior to dosing. The prepared suspension formulation was administered via oral administration to animals (n=3) with 2 mL/kg to achieve the 10 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

### Results

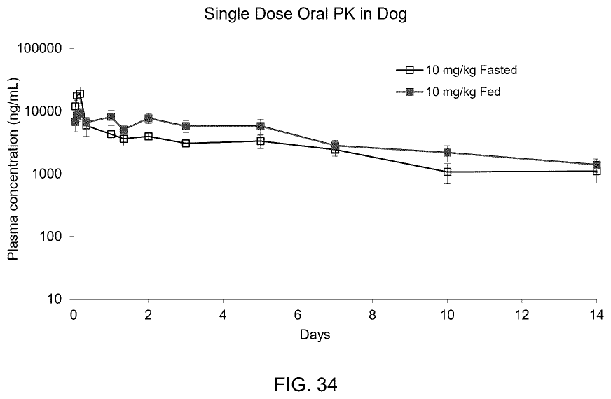

The resulting PK parameters after oral administration Compound 1 SDD suspension are shown in Table 23 and the mean plasma concentration profiles (n=3) are shown in FIG. 34. The Compound 1 SDD suspension achieved immediate high plasma concentrations within 4 hours under both fed and fasted conditions. Remarkably, plasma concentrations remained very high after just a single dose at 10 mg/kg. Plasma levels remained significantly higher than a target level of 1100 ng/ml for at least 5 days and at or higher than target levels for about 14 days. The target plasma concentration was derived from pritelivir, adjusted for Compound 1 protein shift and potency.

TABLE 23
Dog PK Data

| | Dose | Tₘₐₓ | Cₘₐₓ | C_{24 hr} | C_{14 day} | AUC_{0-14 day} |
|---|---|---|---|---|---|---|
| Group | (mg/kg) | (hr) | (ng/ml) | (ng/ml) | (ng/ml) | (hr*ng/ml) |
| 1 | 10 | 4 | 20133 | 4300 | 1103 | 871128 |
| 2 | 10 | 10 | 9660 | 8110 | 1397 | 1311690 |

### Example 13: Preparation of Compound 1 Tablet Compositions

### Preparation of Solid Dispersion for Use in Tablet Formulation

A solid dispersion of Compound 1 using HPMCAS HG was manufactured using a commercial scale spray dryer. Preparation conditions are described in Table 24.

TABLE 24
Spray drying conditions

| | |
|---|---|
| SD composition | 20:80 Compound 1: |
| | HPMCAS HG |
| Batch Size | 12.5 kg |
| Spray solvent | 95:5 THF:Water |
| Spray Solution (wt % total solids) | 1.8 |
| Spray Dryer | GEA PSD-3 |
| Nozzle Type | High Pressure Nozzle |
| Solution Flow Rate (kg/h) | 45 |
| Inlet Temperature (° C.) | 90 |
| Outlet Temperature (° C.) | 50 |
| Secondary Drying Temperature/ | 24 hours at 40° C. |
| Time | |

The solid dispersion was characterised for drug loading and purity and the results from this characterisation work are shown in Table 25.

TABLE 25
Solid dispersion characterisation

| Testing Item | Results |
|---|---|
| Appearance | Off-white powder |
| Assay | 19.3% |
| Total Impurity | 0.22% |
| Residual Solvent- | 297 ppm |
| THF (GC) | |
| XRPD | Amorphous form |
| T_{g} | 103.9° C. |

### Preparation of Tablet Composition

A tablet composition using the solid dispersion was manufactured using a dry granulation method. The composition of the tablet formulation is given in Table 26.

TABLE 26
Tablet composition

| | | 50 mg | |
|---|---|---|---|
| | Component | mg/Tablet | % |
| Intra- | SDD | 250.00 | 62.50 |
| granulation | MCC, Avicel PH 105 | 47.00 | 11.75 |
| | Mannitol, Parteck M100 | 46.00 | 11.50 |
| | Croscarmellose Sodium | 12.00 | 3.00 |
| | Sodium Lauryl Sulfate | 4.00 | 1.00 |
| | Colloidal Silicon Dioxide | 3.00 | 0.75 |
| | Magnesium Stearate | 2.00 | 0.50 |
| Final | MCC, Avicel PH 200 | 20.00 | 5.00 |
| Blending | Croscarmellose Sodium | 12.00 | 3.00 |
| | Colloidal Silicon Dioxide | 1.00 | 0.25 |
| | Magnesium Stearate | 3.00 | 0.75 |
| | Total | 400.00 | 100.00 |

The tablet formulation was prepared as follows.

The SDD was mixed with the intra-granular excipients described in Table 26. Roller compaction was utilized to granulate the mixture. Extra-granular components were then added and blended. The final blend was compressed into tablets. The tablet formulation was characterised and the results from this characterisation are given in Table 27.

TABLE 27
Tablet characterisation

| Item | Results | |
|---|---|---|
| Mean Tablet Weight | 401.53 | mg |
| Tablet Weight RSD | 0.48% | |
| Thickness | 5.5 | mm |
| Hardness | 15.7-18.7 | kp |
| Disintegration Time | 96-109 | sec |

| | |
|---|---|
| Friability | 0.2% |

### Example 14: Single Dose Fed/Fasted Solid Dispersion Tablet PK Study in Dog

### Animal Dosing

Male non-naïve beagles were used for the dog PK study and split into the following three groups:

| | | | | | Stomach pH |
|---|---|---|---|---|---|
| | | | | | before oral |
| | N | Dose | Food | Pretreatment | dosing |
| Group 1 | 3 | 100 mg | Fasted | Pentagastrin | 1.5-2 |
| Group 2 | 3 | 100 mg | Fasted | Famotidine | 6-7 |
| Group 3 | 3 | 100 mg | Fed | None | Not |
| | | | | | measured |

The dogs in Group 1 were pre-treated with pentagastrin to lower the gastric pH. 30 minutes before each administration of dose for Group 1, 6 μg/kg (80 L/kg) of pentagastrin was given via the intra-muscular route to the animals. Stomach pH was measured prior to Compound 1 tablet oral administration. A tube was inserted into the stomach through a catheter, and a small volume of gastric fluid was aspirated through the tubing. The pH of the gastric fluid was determined using a pH meter.

The dogs in Group 2 were pre-treated with famotidine to increase the gastric pH. 60 minutes before each administration of dose for Group 2, 40 mg/dog of Famotidine tablet was given orally to animals. Stomach pH was measured prior to Compound 1 tablet oral administration. A tube was inserted into the stomach through a catheter, and a small volume of gastric fluid was aspirated through the tubing. The pH of the gastric fluid was determined using a pH meter.

Tablets manufactured as described in Example 13 were dosed orally via gavage to achieve 100 mg dose per dog (2×50 mg strength tablets). Water (10 mL) was flushed down the gavage after tablet administration.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS using the following conditions:

### LC-MS Parameters for Compound 1 Quantification in Dog Plasma Samples Following Oral Administration

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 | | |
| | (2.1 × 50 mm, 2.5 μm) | | |
| Flow rate | 0.60 mL/min | | |
| Column Temperature | 60° C. | | |
| Sample diluent | MeOH:H₂O 50:50 (v:v) | | |
| Mobile Phase A | Water/0.025% Formic acid/ | | |
| | 1 mM Ammonium Acetate | | |
| Mobile Phase B | Methanol/0.025% Formic acid/ | | |
| | 1 mM Ammonium Acetate | | |
| Program Type | Gradient | | |
| | | % Mobile | % Mobile |
| Gradient Program | Time (min) | Phase A | Phase B |
| | 0.0 | 90 | 10 |
| | 0.20 | 90 | 10 |
| | 0.40 | 40 | 60 |
| | 1.80 | 10 | 90 |
| | 1.81 | 90 | 10 |
| | 2.20 | Stop | Stop |

| | |
|---|---|
| Sample Temperature | Room Temperature |
| Injection Volume | 1.0 μL |
| MS Detection Mode | Multiple reaction monitoring |
| | (MRM) |
| Retention time | 1.21 min |

### Results

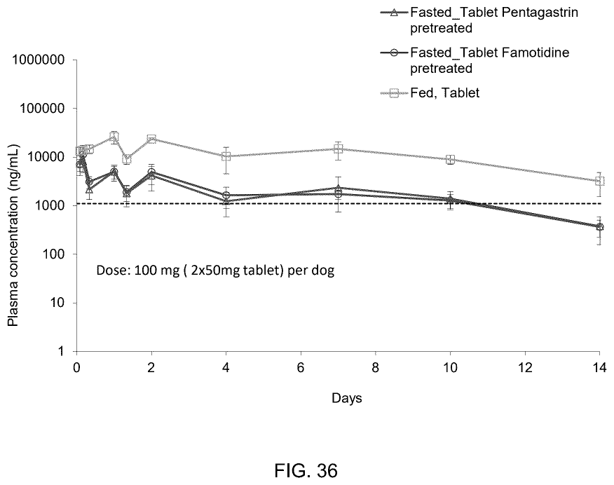

The resulting PK parameters after oral administration of the tablet formulations are shown in Table 28 and the mean plasma concentration profiles (n=3) are shown in FIG. 36. The tablet formulations dosed in the fasted state, with pentagastrin or famotidine pre-treatment, achieved rapid plasma concentrations. Remarkably, plasma concentrations remained high after just a single dose. Plasma levels remained significantly higher than a target level of 1100 ng/ml for at least 5 days. The target plasma concentration was derived from pritelivir, adjusted for Compound 1 protein shift and potency. The results indicate that the pharmacokinetic behaviour of the tablet formulations following oral administration to dogs is resistant to differences in gastric pH.

The data from Group 3 indicates that exposure is enhanced in the presence of food. Plasma concentrations remained high after just a single dose. Plasma levels remained significantly higher than a target level of 1100 ng/ml for at least 14 days.

TABLE 28
Dog PK Data

| | Dose | Tₘₐₓ | Cₘₐₓ | C_{24 hr} | C_{14 day} | AUC_{0-14 day} |
|---|---|---|---|---|---|---|
| | (mg/dog) | (hr) | (ng/ml) | (ng/ml) | (ng/ml) | (hr*ng/ml) |
| Group 1 | 100 | 4 | 8303 | 5010 | 376 | 607492 |
| Group 2 | 100 | 3.33 | 11300 | 4987 | 364 | 627829 |
| Group 3 | 100 | 32 | 28467 | 26167 | 3187 | 3821060 |

Comparison of Tablet with SDD Suspension in Fasted State

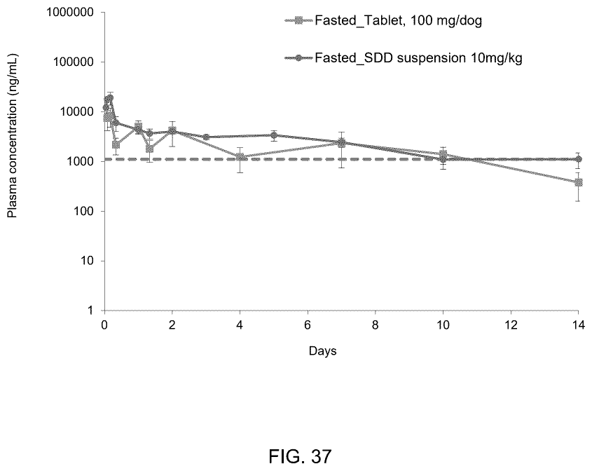

The mean plasma concentration profile (n=3) of the tablet formulation in the fasted state, compared to the SDD suspension formulation dosed at 10 mg/kg in the fasted state as described in Example 12, is shown in FIG. 37. The data shows that the tablets were able to provide comparable exposure to the SDD suspension in the fasted state.

Comparison of Tablet with SDD Suspension in Fed State

The SDD suspension formulation described in Example 12 was dosed to male non-naïve beagles in the fed state. The animals were given a meal 0.5 to 1 hour before dosing. The prepared suspension formulation was administered via oral administration to animals (n=3) with 2 mL/kg to achieve the 10 mg/kg dose.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS.

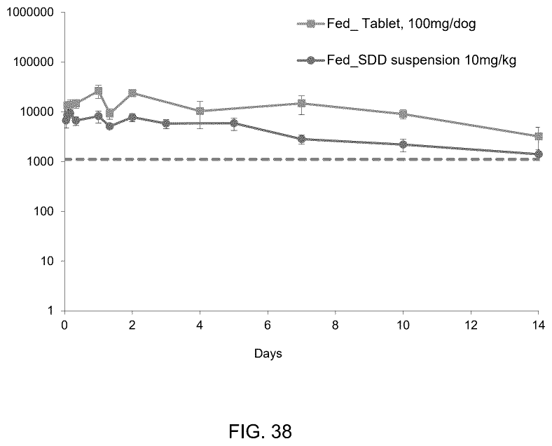

The mean plasma concentration profile (n=3) of the tablet formulation in the fed state (Group 3), compared to the SDD suspension formulation dosed at 10 mg/kg as in the fed state, is shown in FIG. 38. The data indicates that exposure is increased in the presence of food for both formulations. Plasma concentrations remained high after just a single dose. Plasma levels remained significantly higher than a target level of 1100 ng/ml for at least 14 days.

### Example 15: Preparation and Testing of Further Compound 1 Solid Dispersions

The following solid dispersions containing Compound 1 were prepared using a spray drying method:
Example 15O—Compound 1 spray dried without polymer (No polymer)
Example 15A—20:80 Cpd 1: Eudragit® L100 SD
Example 15B—20:80 Cpd 1: Eudragit® E100 SD
Example 15C—20:80 Cpd 1: Soluplus® SD
Example 15D—20:80 Cpd 1: PVP VA64 SD
Example 15E—20:80 Cpd 1: HPMC E3 SD
Example 15F—20:80 Cpd 1: HPMCAS-LG SD
Example 15G—20:80 Cpd 1: HPMCAS-MG SD
Example 15H—20:80 Cpd 1: HPMCAS-HG SD

All formulations were spray dried from 95:5 THF: water. A secondary tray drying process was used to remove residual solvent after the initial spray drying process. In this operation, the “wet” SDI was heated to 40° C. and stored in a convection tray oven for roughly 66 hours. A summary of spray drying parameters and recovered yields are shown in Table 29.

TABLE 29
Summary of Spray Drying Parameters for Cpd 1 SDs

| Formulation | 15O | 15A | 15B | 15C | 15D | 15E | 15F | 15G | 15H |
|---|---|---|---|---|---|---|---|---|---|
| Spray Solvent | 95:5 THF:Water | | | | | | | | |
| Spray Solution | 2% | | | | | | | | |
| Solids Content | | | | | | | | | |
| (wt %) | | | | | | | | | |
| Nozzle Type | Two-Fluid-1.5 mm Air Cap, 0.7 mm Liquid Tip | | | | | | | | |
| Pump rate | 30% | | | | | | | | |
| Q-flow | 28% | 25% | 28% | 25% | 28% | 28% | 28% | 25% | 25% |
| Inlet Temp. (° C.) | 118- | 122- | 119- | 119- | 118- | 117- | 120- | 119- | 119- |
| | 121 | 123 | 121 | 121 | 12 | 120 | 121 | 12 | 120 |
| Outlet Temp. (° C.) | 69-71 | 74-77 | 74-77 | 75-78 | 75-78 | 76-79 | 73-75 | 77-78 | 78-79 |
| Dry SD Yield (wt %) | 54.2 | 54.7 | 40.6 | 66.2 | 34.9 | 65.1 | 45.2 | 44.0 | 62.9 |

All SD formulations were obtained as off-white powders. Thermal analysis by MDSC showed that all dispersions had a single Tg, indicative of an intimately mixed amorphous solid dispersion with good homogeneity.

The preliminary dissolution performance of the SD samples and bulk Compound 1 was tested in a biorelevant non-sink dissolution experiment. About 0.5 mg SD samples were dispersed into 6 mL SGF medium firstly and stirred magnetically with 250 rpm at 37° C. After 30 min, 5 mL cFaSSIF-V2 (2×) medium was added into the above medium and adjusted the pH to 6.5 with 100 μL NaHCO₃ (80 g/L). At each time point of 15 and 30 min in SGF and 15, 30, 60, 120 min in FaSSIF, 0.5 mL suspension was taken out and centrifuged. The supernatants were analyzed by HPLC.

All SD formulations provided an increase in drug dissolution and a sustainment in intestinal media, with the 20% Cpd 1 in Soluplus (Ex. 15C) and HPMCAS-H (Ex. 15H) showing the highest dissolution performance. Dissolution results are shown in Table 30.

TABLE 30
Summary of MDSC and dissolution testing data for Examples

| | | | Concentration (μg/mL) | | | | | |
|---|---|---|---|---|---|---|---|---|
| | | | In SGF (pH 1.7) | | In FaSSIF (pH 6.5) | | | |
| | | T_{g} | 15 | 30 | 45 | 60 | 90 | 150 |
| Ex. | Formulations | (° C.) | min | min | min | min | min | min |
| | Neat API | — | 0.10 | 0.07 | 0.14 | 0.12 | 0.14 | 0.12 |
| 15O | API SD | 95.5 | 0.64 | 0.42 | 0.26 | 0.27 | 0.28 | 0.46 |
| 15A | 20:80 Cpd 1:Eudragit ® L100 SD | 111.0 | 0.74 | 0.44 | 0.33 | 0.30 | 0.28 | 0.28 |
| 15B | 20:80 Cpd 1:Eudragit ® E100 SD | 58.4 | 0.49 | 0.16 | 0.09 | 0.08 | 0.06 | 0.06 |
| 15C | 20:80 Cpd 1:Soluplus ® SD | 79.3 | 1.35 | 1.16 | 3.03 | 3.42 | 3.74 | 3.94 |
| 15D | 20:80 Cpd 1:PVP VA64 SD | 98.0 | 2.77 | 1.63 | 0.57 | 0.72 | 0.85 | 1.45 |
| 15E | 20:80 Cpd 1:HPMC E3 SD | 109.9 | 0.36 | 0.23 | 0.50 | 0.38 | 0.40 | 0.48 |
| 15F | 20:80 Cpd 1:HPMCAS-LG SD | 100.5 | 0.66 | 0.27 | 0.59 | 0.65 | 0.53 | 0.54 |
| 15G | 20:80 Cpd 1:HPMCAS-MG SD | 98.1 | 0.49 | 0.34 | 1.31 | 0.98 | 0.85 | 1.27 |
| 15H | 20:80 Cpd 1:HPMCAS-HG SD | 97.6 | 0.39 | 0.37 | 1.99 | 2.67 | 2.42 | 1.64 |

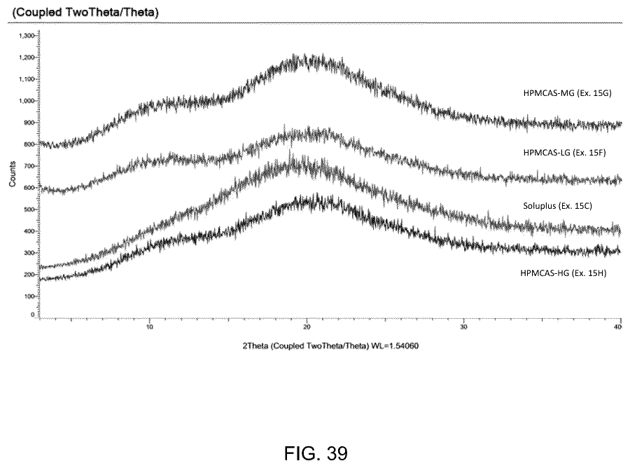

The SDs in Soluplus (Ex. 15C), HPMCAS-LG (Ex. 15F), HPMCAS-MG (Ex. 15G) and HPMCAS-HG (Ex. 15H) were further characterized by GC, XPRD and HPLC. XPRD diffractograms are shown in FIG. 39 and show that all SDDs were amorphous. The GC, assay and purity results are summarized in Table 31.

TABLE 31
Characterisation of SDs

| | Ex. | | | |
|---|---|---|---|---|
| | 15C | 15F | 15G | 15H |
| | 20:80 Cpd | 20:60 Cpd | 20:80 Cpd | 20:80 Cpd |
| | 1:Soluplus ® | 1:HPMCAS- | 1:HPMCAS- | 1:HPMCAS- |
| Formulation | SD | LG SD | MG SD | HG SD |
| GC (THF, ppm) | 716 | 81 | 114 | 64 |
| Assay (%) | 20.4 | 19.3 | 20.2 | 20.1 |
| Purity (%) | 99.73 | 99.73 | 99.72 | 99.61 |

### Stress Stability of Compound 1 Solid Dispersions

To assess the physical and chemical stability of Compound 1 SDD formulations, four dispersions—Example 15C (20:80 Cpd 1: Soluplus), Example 15F (20:80 Cpd 1: HPMCAS-LG), Example 15G (20:80 Cpd 1: HPMCAS-MG) and Example 15H (20:80 Cpd 1: HPMCAS-HG) were aged for 5 days under 40° C./75% RH in open packaging. Approximately 0.5 g of each sample was placed into an open HDPE bottle without a cap and cotton batting was placed in the neck of the bottle.

The SDs were evaluated for physical and chemical stability by appearance, amorphous character by XRPD, assay and impurities by HPLC, after aging.

Appearance testing showed all stability samples except SD in soluplus were observed to contain freely flowing off-white powder after aging. 20:80 Cpd 1: Soluplus® SD sample was observed to form as a lump and stick on the bottom of the bottle.

Assay and purity data is shown in Table 32.

TABLE 32
Characterisation of SDs

| | | | Assay | Purity | Impurities (%) | | | |
|---|---|---|---|---|---|---|---|---|
| Ex | Formulation | Condition | (%) | (%) | RRT0.56 | RRT0.80 | RRT1.36 | RRT1.72 |
| 15C | 20:80 Cpd | Initial | 20.4 | 99.73 | / | 0.09 | 0.07 | 0.10 |
| | 1:Soluplus ® | | | | | | | |
| | SD | 40 C./75% | 19.1 | 99.73 | / | 0.10 | 0.07 | 0.10 |
| | | open, 5 d | | | | | | |
| 15F | 20:80 Cpd | Initial | 19.3 | 99.73 | / | 0.10 | 0.07 | 0.11 |
| | 1:HPMCA | | | | | | | |
| | S-LG SD | 40 C./75% | 19.5 | 99.73 | / | 0.09 | 0.07 | 0.11 |
| | | open, 5 d | | | | | | |
| 15G | 20:80 Cpd | Initial | 20.2 | 99.72 | / | 0.09 | 0.08 | 0.10 |
| | 1:HPMCA | | | | | | | |
| | S-MG SD | 40 C./75% | 19.9 | 99.72 | / | 0.10 | 0.07 | 0.11 |
| | | open, 5 d | | | | | | |
| 15H | 20:80 Cpd | Initial | 20.1 | 99.61 | 0.11 | 0.10 | 0.07 | 0.11 |
| | 1:HPMCA | | | | | | | |
| | S-HG SD | 40 C./75% | 19.7 | 99.61 | 0.11 | 0.10 | 0.07 | 0.11 |
| | | open, 5 d | | | | | | |

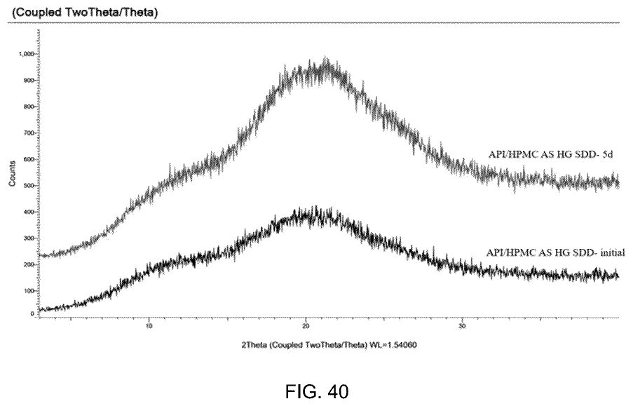

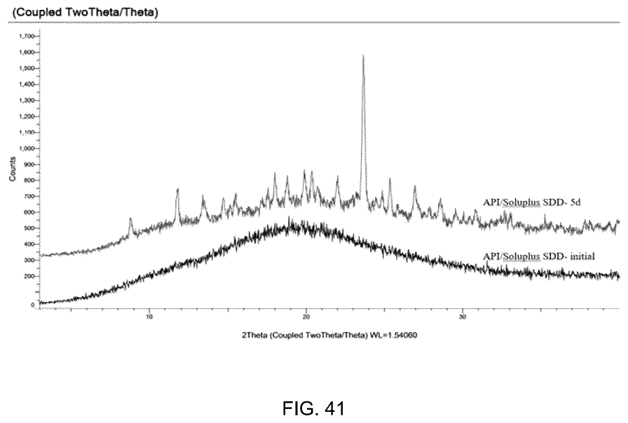

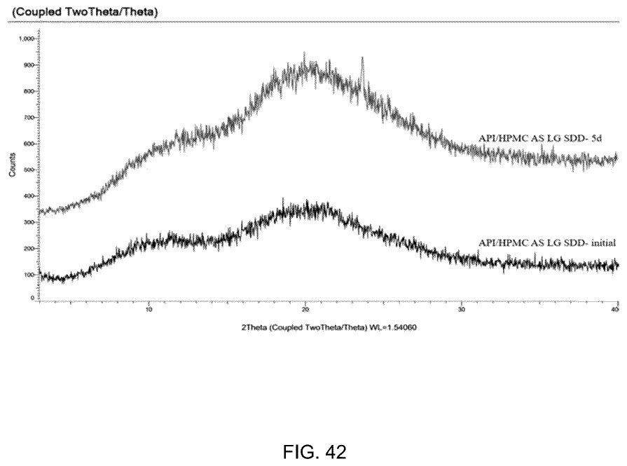

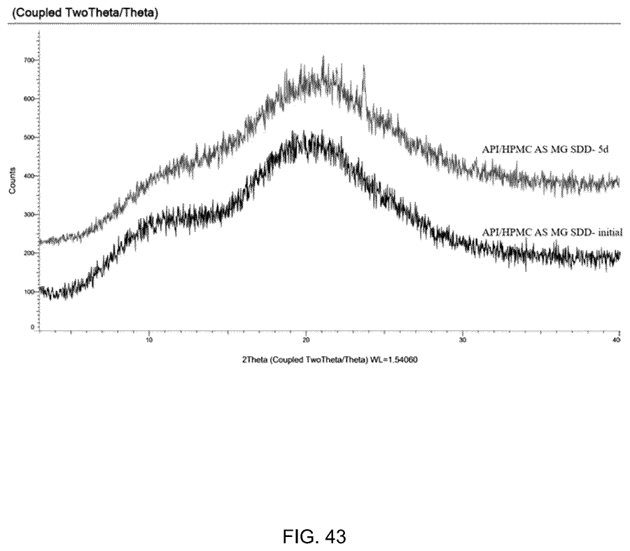

After stressed stability at 40° C./75% RH open conditions for five days, XRPD analysis of the aged SDD samples showed that 20:80 Cpd 1: HPMCAS-HG sample remained amorphous, with no detectable crystalline material (FIG. 40). 20:80 Cpd 1: Soluplus® SD showed obvious signs of crystalline material after aging at 40° C./75% RH open conditions for five days (FIG. 41). 20:80 Cpd 1: HPMCAS-LG (FIG. 42) and 20:80 Cpd 1: HPMCAS-MG (FIG. 43) samples showed a few small crystalline peaks after aging at 40° C./75% RH open conditions for five days.

### In Vitro Test of Compound 1 Solid Dispersions by Pion μFlux Device

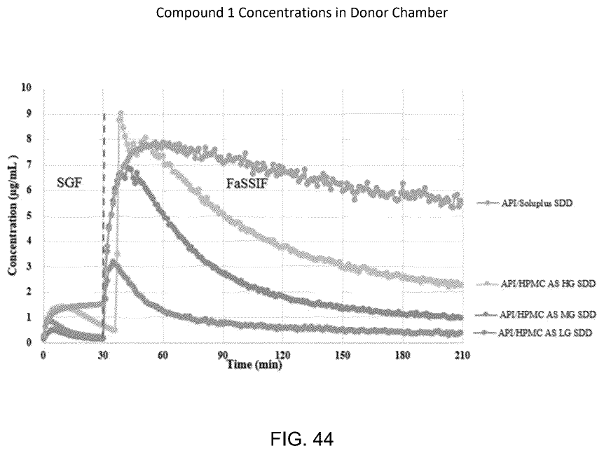

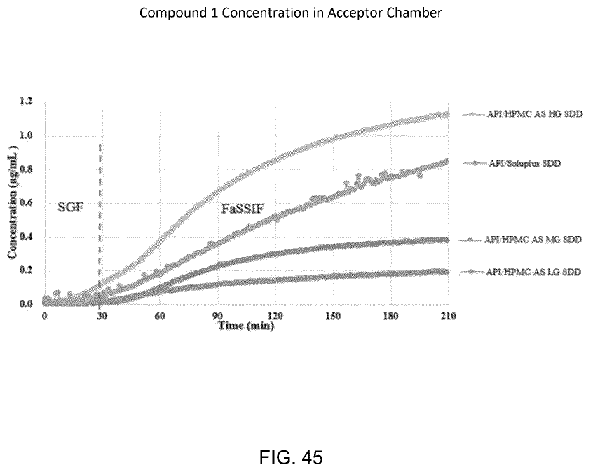

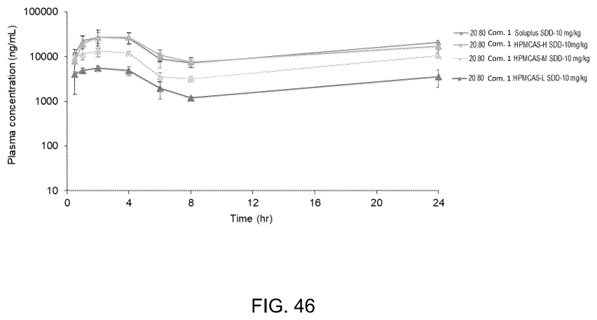

SDs in Soluplus (Ex. 15C), HPMCAS-LG (Ex. 15F), HPMCAS-MG (Ex. 15G) and HPMCAS-HG (Ex. 15H) dissolution and permeation were further evaluated using a PION μflux device. About 20 mL ASB (Acceptor Sink Buffer, pH 7.4, Pion Inc) was added into acceptors firstly. Then about 5 mg solid dispersions were dispersed into 16 mL SGF medium in donor chamber and stirred magnetically with 250 rpm at 37° C. The donor and acceptor chamber were connected using semipermeable membrane. After 30 min, 4 mL concentrated FaSSIF medium was added into donor chamber to reach target API concentration of 50 μg/mL for 3 h. The data was collected with 1 point/min. The donor and acceptor chamber results are shown in FIGS. 44 and 45.

### Example 16: Single Dose Oral Compound 1 PK Study in Dog

Four dispersions—Example 15C (20:80 Cpd 1: Soluplus), Example 15F (20:80 Cpd 1: Soluplus), Example 15G (20:80 Cpd 1: HPMCAS-MG) and Example 15H (20:80 Cpd 1: HPMCAS-HG) were dosed orally in dogs to evaluate PK.

### Formulation Preparation: SDD Suspension

SDD in 0.5% HPMC in Water at 125 mg/ml (drug concentration of 25 mg/mL) were prepared as follows:
1) 1800 mg SDD was weighed into an appropriate sized mortar.
2) 2 mL of HPMC solution was added slowly. While adding, pestle was used to intimately mix and wet the powder to get a wet paste
3) 70 mL 0.5% HPMC in water was added slowly and the pestle was used to break up powder agglomeration to achieve a suspension
4) Suspension was transferred to an appropriate vessel.

The final dosing formulation was a white homogenous suspension. The suspension was stirred prior to dosing.

### Animal Dosing

Male non-naïve beagles were used for the dog PK study. The animals were fasted overnight before dosing. The prepared suspension formulations were administered via oral administration to each animal group (n=3) with appropriate dosing volume to achieve the respective doses.

At each time point, approximately 0.5 mL of whole blood was collected into K2EDTA tubes. Blood samples were put on ice and centrifuged at 2000 g for 5 min to obtain plasma samples within 15 minutes. Plasma samples were stored at approximately −70° C. until analyzed by LC-MS using the following conditions:

### LC-MS Parameters for Compound 1 Quantification in Dog Plasma Samples Following Oral Administration

| Parameter | Value | | |
|---|---|---|---|
| Instrument | LC-MS/MS-47 (Triple Quad 6500+) | | |
| MS conditions | Positive, ESI | | |
| Column | ACQUITY UPLC-BEH C18 | | |
| | (2.1 × 50 mm, 2.5 μm) | | |
| Flow rate | 0.60 mL/min | | |
| Column Temperature | 60° C. | | |
| Sample diluent | MeOH: H₂O 50:50 (v:v) | | |
| Mobile Phase A | Water/0.025% Formic acid/ | | |
| | 1 mM Ammonium Acetate | | |
| Mobile Phase B | Methanol/0.025% Formic acid/ | | |
| | 1 mM Ammonium Acetate | | |
| Program Type | Gradient | | |
| | | % Mobile | % Mobile |
| Gradient Program | Time (min) | Phase A | Phase B |
| | 0.0 | 90 | 10 |
| | 0.20 | 90 | 10 |
| | 0.40 | 40 | 60 |
| | 1.80 | 10 | 90 |
| | 1.81 | 90 | 10 |
| | 2.20 | Stop | Stop |

| | |
|---|---|
| Sample Temperature | Room Temperature |
| Injection Volume | 1.0 μL |
| MS Detection Mode | Multiple reaction monitoring |
| | (MRM) |
| Retention time | 1.21 min |

### Results

The dosing amounts and resulting PK parameters after oral administration of the suspensions prepared with the four different SDDs are shown in Table 33 and the mean plasma concentration profiles (n=3) are shown in FIG. 46.

TABLE 33
Dog dosing and PK Data

| | | Dose | Cₘₐₓ | AUC(0-24 hr) |
|---|---|---|---|---|
| Ex. | Formulation | (mg/kg) | (ng/ml) | (hr*ng/ml) |
| 15C | 20:80 Cpd 1: | 10 | 28367 | 340832 |
| | Soluplus ® SD | | | |
| 15F | 20:80 Cpd 1: | 10 | 5697 | 62206 |
| | HPMCAS-LG SD | | | |
| 15G | 20:80 Cpd 1: | 10 | 14300 | 170070 |
| | HPMCAS-MG SD | | | |
| 15H | 20:80 Cpd 1: | 10 | 30067 | 326889 |
| | HPMCAS-HG SD | | | |

1. An oral pharmaceutical composition comprising a solid dispersion, wherein the solid dispersion comprises a compound (Compound 1) represented by: 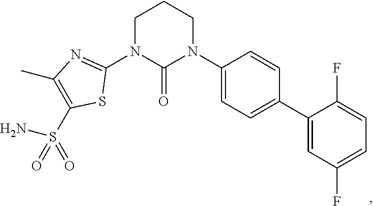 [SMILES: Cc1nc(N2CCCN(c3ccc(-c4cc(F)ccc4F)cc3)C2=O)sc1S(N)(=O)=O]
or a pharmaceutically acceptable salt thereof, and at least one matrix polymer.

2. A composition according to claim 1, wherein the solid dispersion is a substantially amorphous solid dispersion.

3. A composition according to claim 1, wherein the solid dispersion is an amorphous solid dispersion.

4. A composition according to claim 1, wherein the solid dispersion has a single glass transition temperature (T_{g}).

5. A composition according to claim 1, wherein Compound 1 is in its neutral free form.

6. A composition according to claim 1, wherein the solid dispersion comprises about 5 wt % to about 60 wt % of Compound 1 or a pharmaceutically acceptable salt thereof.

7. A composition according to claim 1, wherein the solid dispersion comprises about 30 wt % to about 95 wt % of the at least one matrix polymer.

8. A composition according to claim 1, wherein the solid dispersion comprises about 5 wt % to about 20 wt % of compound 1 or a pharmaceutically acceptable salt thereof and about 60 wt % to about 95 wt % of the at least one matrix polymer.

9. A composition according to claim 1, wherein the at least one matrix polymer is a water insoluble or water soluble ionic or neutral polymer.

10. A composition according to claim 9, wherein the at least one matrix polymer is:
i. an ionic polymer or a pH-sensitive polymer, optionally wherein the pH-sensitive polymer dissolves in aqueous media at above about pH 5.5;
ii. selected from a povidone polymer, a copovidone polymer, a methacrylate polymer, a polymethacrylate-based copolymer, poly(vinyl caprolactam-co-vinyl acetate-co-ethylene glycol), hydroxypropyl methylcellulose, hydroxypropyl methylcellulose acetate succinate and hydroxypropyl methylcellulose phthalate;
iii. selected from a copovidone polymer, poly(vinyl caprolactam-co-vinyl acetate-co-ethylene glycol), hydroxypropyl methylcellulose acetate succinate and hydroxypropyl methylcellulose phthalate; or
iv. selected from hydroxypropyl methylcellulose acetate succinate and hydroxypropyl methylcellulose phthalate.

11. A composition according to claim 1, wherein there is only one matrix polymer present within the solid dispersion.

12. A composition according to claim 1, wherein the solid dispersion is a spray-dried solid dispersion.

13. A composition according to claim 1, wherein the pharmaceutical composition is in a dosage form suitable for oral administration, selected from the group consisting of a granule, a pellet, a tablet, a particle, a capsule, a suspension and a mini-tablet.

14. A composition according to claim 13, wherein the pharmaceutical composition is a tablet with a total weight of less than 1000 mg, such as less than 900 mg, or less than 600 mg.

15. A composition according to claim 13, wherein the pharmaceutical composition comprising about 1 mg to about 500 mg of Compound 1 or a pharmaceutically acceptable salt thereof.

16. A composition according to claim 1, wherein the composition is chemically and/or physically stable for at least four weeks.

17. A composition according to claim 1, wherein the composition, after oral administration to a human subject in the fasted state, produces a plasma concentration in the subject after administration of Compound 1 of at least 1100 ng/ml for at least 80% of the dosing interval, wherein the dosing interval is at least 2 days.

18. (canceled)

19. A method for treating a herpes virus (such as HSV) infection in a subject in need thereof, the method comprising administering a therapeutically effective amount Compound 1, or a pharmaceutically acceptable salt thereof, to the subject wherein the Compound 1 or a pharmaceutically acceptable salt thereof, is formulated as a pharmaceutical composition according to claim 1.

20. The method of claim 19, wherein the pharmaceutical composition is administered no more than once every 5 days, no more than once every 7 days, no more than once every 10 days, twice a month, once a month, once every two months, once every three months, once every six months, or once every year.

21. A method of increasing the bioavailability of Compound 1, or a pharmaceutically acceptable salt thereof, compared to oral administration of an equivalent dose of Compound 1, or a pharmaceutically acceptable salt thereof in a crystalline form, the method comprising orally administering to a subject a pharmaceutical composition according to claim 1.

22. A solid dispersion, wherein the solid dispersion comprises:
a. Compound 1 or a pharmaceutically acceptable salt thereof;
b. at least one matrix polymer; and
c. optionally one or more pharmaceutically acceptable excipients.
